(19) United States
(12) Patent Application Publication
(10) Pub. No.: US 2025/0099492 A1
(43) Pub. Date: Mar. 27, 2025

(54) COMPOSITIONS FOR THE IMPROVED TREATMENT OF ACNE AND RELATED DISORDERS

(71) Applicant: Journey Medical Corporation, Scottsdale, AZ (US)

(72) Inventors: Dov Tamarkin, Maccabim (IL); Elana Gazal, Rehovot (IL); Rita Keynan, Rehovot (IL); Meir Eini, Ness Ziona (IL); David Schuz, Gimzu (IL)

(21) Appl. No.: 18/626,505

(22) Filed: Apr. 4, 2024

Related U.S. Application Data
(63) Continuation of application No. 17/936,562, filed on Sep. 29, 2022, now abandoned.
Application No. 17/936,562 is a continuation of application No. 17/025,156, filed on Sep. 18, 2020, now abandoned.
Application No. 17/025,156 is a continuation of application No. 14/147,376, filed on Jan. 3, 2014, now abandoned.
Application No. 14/147,376 is a continuation-in-part of application No. 13/831,396, filed on Mar. 14, 2013, now Pat. No. 9,849,142.
Application No. 13/831,396 is a continuation-in-part of application No. PCT/IB2013/001170, filed on Mar. 14, 2013.
Application No. PCT/IB2013/001170 is a continuation-in-part of application No. 13/499,501, filed on Sep. 10, 2012, now Pat. No. 8,945,516.
Application No. 13/499,501 is a continuation-in-part of application No. 13/100,724, filed on May 4, 2011, now Pat. No. 8,618,081.
Application No. 13/100,724 is a continuation-in-part of application No. PCT/IB2010/002612, filed on Oct. 1, 2010.
Application No. 13/100,724 is a continuation-in-part of application No. PCT/IB2010/002617, filed on Oct. 1, 2010.
Application No. 13/100,724 is a continuation-in-part of application No. PCT/IB2010/002613, filed on Oct. 1, 2010.
Application No. 14/147,376 is a continuation-in-part of application No. 13/499,475, filed on Sep. 14, 2012, now Pat. No. 8,871,184.
Application No. 14/147,376 is a continuation-in-part of application No. 13/499,709, filed on Sep. 10, 2012, now Pat. No. 10,029,013.
(60) Provisional application No. 61/611,232, filed on Mar. 15, 2012.
(60) Provisional application No. 61/748,603, filed on Jan. 3, 2013.
(60) Provisional application No. 61/780,074, filed on Mar. 13, 2013.
(60) Provisional application No. 61/779,953, filed on Mar. 13, 2013.
(60) Provisional application No. 61/831,981, filed on Jun. 6, 2013.
(60) Provisional application No. 61/831,795, filed on Jun. 6, 2013.
(60) Provisional application No. 61/248,144, filed on Oct. 2, 2009.
(60) Provisional application No. 61/322,148, filed on Apr. 8, 2010.
(60) Provisional application No. 61/331,126, filed on May 4, 2010.
(60) Provisional application No. 61/349,911, filed on May 31, 2010.
(60) Provisional application No. 61/380,568, filed on Sep. 7, 2010.
(60) Provisional application No. 61/385,385, filed on Sep. 22, 2010.
(60) Provisional application No. 61/388,884, filed on Oct. 1, 2010.
(60) Provisional application No. 61/248,144, filed on Oct. 2, 2009.
(60) Provisional application No. 61/322,148, filed on Apr. 8, 2010.
(60) Provisional application No. 61/349,911, filed on May 31, 2010.
(60) Provisional application No. 61/385,385, filed on Sep. 22, 2010.
(60) Provisional application No. 61/331,126, filed on May 4, 2010.
(60) Provisional application No. 61/388,884, filed on Oct. 1, 2010.
(60) Provisional application No. 61/380,568, filed on Sep. 7, 2010.
(60) Provisional application No. 61/248,144, filed on Oct. 2, 2009.
(60) Provisional application No. 61/322,148, filed on Apr. 8, 2010.
(60) Provisional application No. 61/349,911, filed on May 31, 2010.
(60) Provisional application No. 61/385,385, filed on Sep. 22, 2010.
(60) Provisional application No. 61/331,126, filed on May 4, 2010.
(60) Provisional application No. 61/388,884, filed on Oct. 1, 2010.
(60) Provisional application No. 61/380,568, filed on Sep. 7, 2010.
(60) Provisional application No. 61/248,144, filed on Oct. 2, 2009.
(60) Provisional application No. 61/322,148, filed on Apr. 8, 2010.
(60) Provisional application No. 61/331,126, filed on May 4, 2010.
(60) Provisional application No. 61/349,911, filed on May 31, 2010.
(60) Provisional application No. 61/380,568, filed on Sep. 7, 2010.
(60) Provisional application No. 61/385,385, filed on Sep. 22, 2010.
(60) Provisional application No. 61/388,884, filed on Oct. 1, 2010.

(51) Int. Cl. A61K 31/65; A61K 9/00; A61K 9/06; A61K 9/12; A61K 47/06; A61K 47/10; A61K 47/12; A61K 47/24; A61K 47/44

(52) U.S. Cl. CPC A61K 31/65; A61K 9/0014; A61K 9/06; A61K 9/122; A61K 47/10; A61K 47/12; A61K 47/44; A61K 47/06; A61K 47/24

(57) ABSTRACT

Methods of treatment and dosage regimes using hydrophobic gel or foam composition comprising a tetracycline antibiotic in treating or alleviating a disorder including, acne, acne related symptoms, a tetracycline antibiotic responsive acne related disorder, skin disorder caused by a bacteria, and a tetracycline antibiotic responsive sebaceous gland disease, P. acne bacteria associated disorders and other superficial infections, including skin infections wherein the foam composition or gel is administered topically to a target area on a subject having the disorder and wherein the target area comprises an area of skin.

### CROSS REFERENCE TO RELATED APPLICATIONS

This application is a Continuation of U.S. application Ser. No. 17/936,562, filed Sep. 29, 2022, which is a Continuation of U.S. application Ser. No. 17/025,156, filed Sep. 18, 2020, which is a Continuation of U.S. application Ser. No. 14/147,376, filed Jan. 3, 2024, which claims the benefit of priority under 35 U.S.C. § 119(e) to U.S. Provisional Application No. 61/611,232, filed Mar. 15, 2012, U.S. Provisional Application No. 61/748,603, filed Jan. 3, 2013, U.S. Provisional Application No. 61/780,074, filed Mar. 13, 2013, U.S. Provisional Application No. 61/779,953 filed Mar. 13, 2013, U.S. Provisional Application No. 61/831,981, filed Jun. 6, 2013, and U.S. Provisional Application No. 61/831,795, filed Jun. 6, 2013; and is a continuation in part application of:
1) U.S. patent application Ser. No. 13/831,396, filed Mar. 14, 2013, and International Patent Application No. PCT/IB2013/001170, filed Mar. 14, 2013;
2) U.S. patent application Ser. No. 13/499,501, filed Sep. 10, 2012, which is a 371 of International Patent Application No. PCT/IB2010/02612 filed Oct. 1, 2010, which claims the benefit of priority under 35 U.S.C. § 119(e) to U.S. Provisional Application No. 61/248,144, filed Oct. 2, 2009, U.S. Provisional Application No. 61/322,148, filed Apr. 8, 2010, U.S. Provisional Application No. 61/349,911, filed May 31, 2010, U.S. Provisional Application No. 61/385,385, filed Sep. 22, 2010, U.S. Provisional Application No. 61/331,126, filed May 4, 2010, and U.S. Provisional Application No. 61/380,568 filed Sep. 7, 2010;
3) U.S. patent application Ser. No. 13/499,727, filed Sep. 10, 2012, which is a 371 of International Patent Application No. PCT/IB2011/01374, filed May 4, 2011, International Patent Application No. PCT/IB2010/002617, filed Oct. 1, 2010, International Patent Application No. PCT/IB2010/002612, filed Oct. 1, 2010, and International Patent Application No. PCT/IB/2010/002613, filed Oct. 1, 2010, which claims the benefit of priority under 35 U.S.C. § 119(e) to U.S. Provisional Application No. 61/388,884, filed Oct. 1, 2010, U.S. Provisional Application No. 61/385,385, filed Sep. 22, 2010, U.S. Provisional Application No. 61/380,568, filed Sep. 7, 2010, U.S. Provisional Application No. 61/349,911, filed May 31, 2010, and U.S. Provisional Application No. 61/331,126, filed May 4, 2010;
4) U.S. patent application Ser. No. 13/100,724, filed May 4, 2011, which is a continuation in part application of: A) International Application No. PCT/IB2010/002612, filed Oct. 1, 2010, which claims the benefit of priority to U.S. Provisional Application No. 61/248,144, filed Oct. 2, 2009, U.S. Provisional Application No. 61/322,148, filed Apr. 8, 2010, U.S. Provisional Application No. 61/349,911, filed May 31, 2010, U.S. Provisional Application No. 61/385,385, filed Sep. 22, 2010, U.S. Provisional Application No. 61/331,126, filed May 4, 2010, U.S. Provisional Application No. 61/388,884, filed Oct. 1, 2010, and U.S. Provisional Application No. 61/380,568, filed Sep. 7, 2010; B) U.S. patent application Ser. No. 13/100,724, filed May 4, 2011, which is a continuation in part application of International Application No. PCT/IB2010/002617, filed Oct. 1, 2010, which claims the benefit of priority to U.S. Provisional Application No. 61/248,144, filed Oct. 2, 2009, U.S. Provisional Application No. 61/322,148, filed Apr. 8, 2010, U.S. Provisional Application No. 61/349,911, filed May 31, 2010, U.S. Provisional Application No. 61/385,385, filed Sep. 22, 2010, U.S. Provisional Application No. 61/331,126, filed May 4, 2010, U.S. Provisional Application No. 61/388,884, filed Oct. 1, 2010 and U.S. Provisional Application No. 61/380,568 filed Sep. 7, 2010; and C) U.S. patent application Ser. No. 13/100,724, filed May 4, 2011, which is a continuation in part application of International Application No. PCT/IB2010/002613, filed Oct. 1, 2010, which claims the benefit of priority to U.S. Provisional Application No. 61/248,144 filed, Oct. 2, 2009, U.S. Provisional Application No. 61/322,148, filed Apr. 8, 2010, U.S. Provisional Application No. 61/349,911, filed May 31, 2010, U.S. Provisional Application No. 61/385,385, filed Sep. 22, 2010, U.S. Provisional Application No. 61/331,126, filed May 4, 2010, U.S. Provisional Application No. 61/388,884, filed Oct. 1, 2010, and U.S. Provisional Application No. 61/380,568, filed Sep. 7, 2010;
5) U.S. patent application Ser. No. 13/499,475, filed Sep. 14, 2012, which is a 371 of International Patent Application No. PCT/IB2010/002617, filed Oct. 1, 2010, which claims the benefit of priority under 35 U.S.C. § 119(e) to U.S. Provisional Application No. 61/248,144, filed Oct. 2, 2009, U.S. Provisional Application No. 61/322,148, filed Apr. 8, 2010, U.S. Provisional Application No. 61/349,911, filed May 31, 2010, U.S. Provisional Application No. 61/385,385, filed Sep. 22, 2010, U.S. Provisional Application No. 61/331,126, filed May 4, 2010, U.S. Provisional Application No. 61/380,568, filed Sep. 7, 2010, and U.S. Provisional Application No. 61/388,884, filed Oct. 1, 2010; and
6) U.S. patent application Ser. No. 13/499,709 filed Sep. 10, 2012, which is a 371 of International Patent Application No. PCT/IB2010/002613, filed Oct. 1, 2010, and claims the benefit of priority under 35 U.S.C. § 119(e) to U.S. Provisional Application No. 61/248,144, filed Oct. 2, 2009, U.S. Provisional Application No. 61/322,148, filed Apr. 8, 2010, U.S. Provisional Application No. 61/349,911, filed May 31, 2010, U.S. Provisional Application No. 61/385,385, filed Sep. 22, 2010, U.S. Provisional Application No. 61/331,126, filed May 4, 2010, U.S. Provisional Application No. 61/380,568, filed Sep. 7, 2010, and U.S. Provisional Application No. 61/388,884, filed Oct. 1, 2010;
wherein all of the above are herein incorporated by reference in their entireties.

### BACKGROUND

Acne including acne vulgaris is a very common skin disorder, which afflicts almost all teenagers but is also reported in infants and adults. It affects between 40 to 50 million individuals in the United States alone. The prevalence of adult acne is 3% in men and between 11% and 12% in women. Moderate to severe acne is observed in 14% of acne patients. Acne vulgaris (cystic acne or simply acne) is characterized by areas of skin with seborrhea (scaly red skin), comedones (blackheads and whiteheads), papules (pinheads), pustules (pimples), nodules (large papules) and possibly scarring. Acne vulgaris affects mostly skin with the densest population of sebaceous follicles. These areas include the face, the upper part of the chest, and the back. Severe acne vulgaris is inflammatory, but acne vulgaris can also manifest in non-inflammatory forms.

The lesions are caused by changes in pilosebaceous units, skin structures consisting of a hair follicle and its associated sebaceous gland, changes that require androgen stimulation. Androgen metabolism plays an important role in the control of both sebum excretion and keratinization patterns in the pilosebaceous unit. The enzyme 5-α-testosterone reductase is responsible for the conversion of testosterone to dihydrotestosterone (DHT), and DHT is thought to modulate both keratin formation and sebum production. Malfunctioning of the pilosebaceous unit is also the cause of other common skin disorders, such as seborrhea, dandruff, androgenic alopecia and hirsutism.

Acne is thought to be caused by the interplay of four factors. Excessive sebum production secondary to sebaceous gland hyperplasia is the first abnormality to occur. Subsequent hyperkeratinization of the hair follicle prevents normal shedding of the follicular keratinocytes, which then obstruct the follicle and form an inapparent microcomedo. Lipids and cellular debris soon accumulate within the blocked follicle. This microenvironment encourages colonization of Propionibacterium Acnes (P. acnes), which provokes an immune response through the production of numerous inflammatory mediators. Inflammation is further enhanced by follicular rupture and subsequent leakage of lipids, bacteria, and fatty acids into dermis.

There are three levels of acne: mild, moderate, and severe. Mild acne is characterized by the presence of few to several papules and pustules, but no nodules. Patients with moderate acne have several to many papules and pustules, along with a few to several nodules. With severe acne, patients have numerous or extensive papules and pustules, as well as many nodules. Acne also is classified by type of lesion comedonal, papulopustular, and nodulocystic. Pustules and cysts are considered inflammatory acne.

Many different treatments exist for acne including benzoyl peroxide (BPO), antibiotics, retinoids, antiseborrheic medications, anti-androgen medications, hormonal treatments, acid, alpha, azelaic acid, nicotinamide, and keratolytic soaps. They are believed to work in at least four different ways, including: normalizing shedding into the pore to prevent blockage, killing P. acnes, anti-inflammatory effects and hormonal manipulation. However, the current anti-acne preparations cause significant adverse skin reactions, such as primary skin irritation, hyper pigmentation and photosensitivity as described in detail below.

Treatment is generally directed toward the known pathogenic factors involved in acne, including follicular hyperproliferation, excess seburn, P. acnes, and inflammation. The grade and severity of the acne help in determining which of the following treatments, solely or in combination, is most appropriate. When a topical or systemic antibiotic is used, it may be used in conjunction with BPO to reduce the emergence of resistance.

Mild to moderate acne is currently treated topically, using retinoids, benzoyl peroxide and some antibiotics. Topical retinoids are comedolytic and anti-inflammatory. They normalize follicular hyperproliferation and hyperkeratinization. Topical retinoids reduce the numbers of comedones, but they are marginally effective against inflammatory lesions. Antibiotics such as tetracycline antibiotics are only available orally or by injection. Topical antibiotics are mainly used for their role against P. acnes. BPO products are also effective against. P. acnes. Unfortunately, the safety and efficacy profiles of each of these medications are not satisfactory. In one or more embodiments herein, there are provided new and better topical anti-acne treatments and formulations.

BPO works against the P. acnes bacterium and is a first-line treatment for mild and moderate acne vulgaris due to its effectiveness and mild side-effects (primarily an irritant dermatitis). However, it may cause dryness of the skin, slight redness, occasional peeling and increased photo-sensitivity. Although it dries out the skin and temporarily clears superficial pimples and whiteheads, soon after that the skin's own natural defenses kick in to produce more oil, sometimes even more than before to compensate for the stripping that has occurred. And hence a rebounding action occurs. In one or more embodiments there are provided long-term clear skin non-irritant nondrying formulations.

Topical retinoids, which are related to vitamin A, are medications that normalize the cell life cycle in the follicle lining. This helps prevent the hyperkeratinization of these cells that can create a blockage. This class includes tretinoin (Retin-A), adapalene (Differin), and tazarotene (Tazorac). They are prescribed to treat acne ranging from mild to moderately severe, they affect mainly non-inflammatory lesions and they frequently cause side effects. They can, cause significant irritation of the skin and often cause an initial flare-up of acne and facial flushing. For example, Adapalene's side effects in different studies include erythema (6%-27%) dryness (6%-37%), scaling (4%-63%), pruritus (4%-11%) and burning after application (7%-33%). Retin-A Micro@(tretinoin gel) microsphere, 0.1% and 0.04% uses microspheres to enable inclusion of the active ingredient, tretinoin, in an aqueous gel. The skin of certain individuals may become excessively dry, red, swollen, blistered, more susceptibile to sunlight and to severe irritation on eczematous skin. During the early weeks of therapy, an apparent exacerbation of inflammatory lesions may occur. This may cause discontinuation of therapy.

Commonly used antibiotics either applied topically or taken orally, include erythromycin, clindamycin, and tetracyclines such as minocycline.

Zindaclin 1% Gel (CLINDAMYCIN PHOSPHATE, Crawford Pharmaceuticals) treats only mild to moderate acne and frequently causes side effects. Evoclin® Foam 1%, contains clindamycin phosphate, in a thermolabile hydroethanolic foam vehicle. It is indicated once daily topically for twelve weeks for treating mild to moderate acne. Evoclin® Foam side effects include burning, itching, dryness, redness, oily skin or skin peeling.

Akne-mycin®2% is in the form of an ointment (erythromycin, CORIA LABORATORIES) indicated twice daily may cause erythema and peeling.

Systemic antibiotics are indicated for moderate or severe acne. The most commonly used systemic antibiotics are tetracycline and their derivatives (e.g., minocycline). These agents have anti-inflammatory properties, and they are effective against P. acnes. The more lipophilic antibiotics, such as minocycline and doxycycline, are generally more effective than tetracycline. Greater efficacy may also be due to less P. acnes resistance to minocycline.

Oral tetracycline antibiotics are generally not recommended in the treatment of minor or mild acne, primarily because they cause hyper-pigmentation, erythema and dryness. Oral tetracycline therapy may induce hyperpigmentation in many organs, including nails, bone, skin, eyes, thyroid, visceral tissue, oral cavity (teeth, mucosa, alveolar bone), sclerae and heart valves. Skin and oral pigmentation has been reported to occur independently of time or amount of drug administration, whereas other tissue pigmentation has been reported to occur upon prolonged administration. Skin pigmentation includes diffuse pigmentation as well as over sites of scars or injury. Oral tetracyclines should not be used for pregnant women due to teratogenic effects and also prohibited should not be used in nursing mothers and. So there is a need for topical formulations with tetracyclines which can avoid the side effects seen with oral application.

Minocycline hydrochloride is a bacteriostatic tetracycline antibiotic, which has a broader spectrum than the other members of the group. It reduces the redness, swelling and tenderness or pimples whether it kills the acne bacteria or not. It is currently available as an oral drug under the brand names Dynacin, Minocin, Myrac, Solodyn. It is further available in injectable products for IV administration.

Minocycline has a spectrum of activity and a mode of action similar to that of tetracycline but it is more active against many species including P. Acnes, Staphylococcus aureus, streptococci, Neisseria meningitidis, various enterobacteria, Acinetobacter, Bacteroides, Haemophilus, Nocardia, and some mycobacteria, including M. leprae. Partial cross-resistance exists between minocycline and other tetracyclines but some strains resistant to other drugs of the group remain sensitive to minocycline, perhaps because of better cell-wall penetration.

Minocycline is a tetracycline derivative with uses similar to those of tetracycline. It is also a component of multidrug regimens for the treatment of leprosy and has been used in the prophylaxis of meningococcal infection to eliminate the carrier state, but the high incidence of vestibular disturbances, presumeably resulting from it being given systemically, means that it is not the drug of choice for the latter. It has neuroprotective properties, it is being investigated for motor neurone disease, and for the management of Huntington's chorea. It is used in the treatment of rheumatoid arthritis and in the prevention or treatment of various skin disorders.

Whilst oral minocycline is widely prescribed to moderate to severe acne patients, and can decrease inflammatory acne lesions it does not affect the non-inflammatory lesions. For example SOLODYN® is indicated to treat only inflammatory lesions of non-nodular moderate to severe acne vulgaris in patients 12 years of age and older. Common side effects of oral minocycline include diarrhea, dizziness, drowsiness, indigestion, lightheadedness, loss of appetite, nausea, sore mouth, throat or tongue, and vomiting. Minocycline may also cause severe side effects, including severe allergic reactions, bloody stools, blurred vision, change in the amount of urine produced, fever, chills, sore throat, hearing problems, joint pain, muscle pain or weakness, rectal or genital irritation, red, swollen, blistered, or peeling skin, ringing in the ears, seizures, severe or persistent headache, severe skin reaction to the sun, watery diarrhea, stomach cramps or pain, swollen glands, symptoms of pancreatitis, trouble swallowing, unusual bruising or bleeding, unusual tiredness or weakness, vaginal irritation or discharge, white patches in the mouth and yellowing of the skin or eyes. Due to these side effects, the FDA added oral minocycline in 2009 to its Adverse Event Reporting System (AERS), a list of medications under investigation by the FDA for potential safety issues.

Tetracycline antibiotics, such as tetracycline, oxytetracycline, demeclocycline, doxycycline, lymecycline, meclocycline, methacycline, minocycline, rolitetracycline, chlorotetracycline and tigecycline, are extremely unstable compounds and are sensitive to many formulation excipients (for example, water, short chain alcohols, certain polymers, certain hydrophilic solvents and surfactants). Thus, most tetracyclines, e.g., minocycline and doxycycline, currently exist only in solid oral dosage forms or are given by injection or infusion.

A product that requires a shorter treatment period, has no adverse effects, does not cause skin irritation and treats both inflammatory and non-inflammatory lesions effectively would be advantageous and could improve patient compliance. A tetracycline topical product which is safe for use for pregnant and nursing mothers would also be advantageous and would fulfill a long felt need. Due to the broad spectrum of antibacterial activity of tetracycline antibiotics, especially minocycline and doxycycline, topical treatment comprising tetracycline compositions, which can reduce adverse systemic exposure as well as reduce unwanted side effects of such antibiotics is warranted.

Novel, stable, patient-friendly topical hydrophobic therapeutic breakable gel and foamable compositions comprising tetracycline, without surfactants, have been described, for example in U.S. application Ser. Nos. 13/499,501, 13/499,727, 13/499,475, and 13/499,709, U.S. Publication No. 2011/0281827, WO11/039637, WO11/039638, WO 11/138678 and WO 2011/064631 all of which are herein incorporated in their entirety by reference.

Methods for treatment of impetigo using these topical hydrophobic therapeutic breakable gel and foamable compositions comprising tetracycline have been described, for example in U.S. provisional applications Ser. Nos. 61/748,603, 61/780,074, and 61/611,232 respectively, as well as in PCT/US2013/031387, all of which are herein incorporated in their entirety by reference.

The instability of minocycline was observed and confirmed in a compatibility study with pharmaceutical excipients described, for example, in WO11/039637. The study identified and demonstrated that different hydrophilic solvents were incompatible with minocycline, whereas certain hydrophobic solvents, emollients and waxes revealed compatibility with minocycline. Fatty alcohols, as well as some fatty acids (such as stearic acid, behenic acid, oleic acid and palmitic acid) were found to be compatible with minocycline. Additionally, a few certain surfactants (e.g., sucrose fatty esters) and some additives (aerosil and menthol) were disclosed to be compatible with minocycline, whereas other surface active agents including polysorbates, sorbitan esters of fatty acids, polyoxyethylene alkyl ethers and polyoxyethylene esters of fatty acids were found not to be compatible.

It was further discovered, for example, in WO11/039637 that addition of water caused rapid degradation of minocycline and addition of antioxidants (alpha-tocopherol, BHA/BHT and propyl gallate) did not prevent such degradation. Furthermore, compatible excipients became incompatible in the presence of water and addition of antioxidants did not remedy this result. It was also shown, for example, in WO11/039637 that minocycline has activity that decreases apoptosis and increases cell viability.

It was further found, for example, in WO11/039637, in in-vitro skin delivery studies that enhanced penetration was achieved without the need of adding a hydrophilic solvent and thus degradation of minocycline could be further reduced or prevented. Minocycline was found to have been delivered intradermally at sufficient levels to treat skin infections but did not pass through the skin transdermally and therefore topical application should be essentially free from adverse systemic effects.

As can be seen form the above current anti-acne preparations can cause significant adverse reactions, and their efficacy is limited. For example, oral minocyclines can cause moderate to severe skin and systemic side effects but are not effective against non-inflammatory lesions.

The safety, tolerability and clinical efficacy of topical application of minocycline compositions in a population of moderate to severe acne vulgaris patients is assessed herein. A safe and effective dose and dosing regimen for topical minocycline is described herein. Topical treatment of acne using minocycline compositions described herein was found to be more successful than oral therapy. Moreover, topical minocycline treatment of acne was found to be effective against inflammatory lesions and non-inflammatory lesions. Remarkably, there are provided herein compositions and novel methods of treatment with topical tetracycline antibiotics e.g. minocycline, which are successful in reducing the number of non-inflammatory lesions even though when minocycline is given orally it does not affect non-inflammatory lesions. In one or more embodiments this reduction is statistically significant. In one or more embodiments there is provided a method of treating a subject with non-inflammatory lesions by applying to an area of skin a gel or foam formulation comprising a tetracycline antibiotic. In one or more embodiments the application is alternative days. In one or more embodiments the application is daily. In one or more embodiments the application is two or three or four or five or six times a day, as needed. In one or more embodiments the application is for one two, three, four, five six, seven, eight, nine, ten, eleven, twelve, thirteen, fourteen, fifteen, or sixteen weeks. In one or more embodiments the treatment is for three, four, five or six months.

### SUMMARY

Preliminary results have surprisingly shown that the topical administration of a foamable composition comprising a tetracycline antibiotic, minocycline, once daily provided effective drug delivery to an infected lesion site, leading to rapid reduction in the number of non-inflammatory and inflammatory acne lesions within only six weeks of treatment.

Clinical trial results have now remarkably shown that the topical administration of a foamable composition comprising a tetracycline antibiotic, minocycline, once daily provided effective drug delivery to an infected lesion site, leading to rapid reduction in the number of non-inflammatory and inflammatory acne lesions. A reduction in lesions could be observed from about 3 weeks with improvement continuing to 12 weeks. Inflammatory lesions responded, in general, earlier on in the treatment than non-inflammatory lesions. The effect of treatment on reducing the number of lesions and improving the patient's skin appeared to approach a steady state between 6-12 weeks. Treatment was stopped at twelve weeks but the patients were seen again 4 weeks after cessation of treatment at week 16. Surprisingly, the effect of the previous 12 weeks of treatment on reducing the number of lesions and improving the patient's skin was observed to continue in the absence of treatment with minor or no changes in the mean number of lesions. In other words, four weeks after cessation of treatment the patient's skin did not appear to show signs of relapse.

The 150 patients starting the Clinical study had on average moderate severe to severe acne with a mean number of inflammatory lesions ranging from 33-36 and a mean number of non-inflammatory lesions of 42-46. Accordingly, patients started the study with about 75-82 lesions on average. The effects observed were dose dependent. The results seen with 4% minocycline were better than those with 1% minocycline which in turn were better than the vehicle formulation without minocycline which was used as the placebo (the “placebo formulation” or “placebo”). Nevertheless, the placebo had a substantial and unexpected positive effect. For example, as described below, following daily application a substantial reduction of acne lesions was observed. The effect of the vehicle without minocycline may be a contributing factor in the success observed with the 1% and 4% minocycline formulations as it may act as a springboard or platform from which the antibiotic can have its therapeutic effect. By having an effective vehicle topical minocycline can achieve more for the skin than oral minocycline. Without being bound by any theory, it is thought that the selection of excipients that are compatible with minocycline may have contributed to the effect of the placebo formulation. One or more of the excipients in the vehicle may have a therapeutic effect on their own and may act together with the tetracycline to amplify its effect and thereby achieving two incremental advantages over oral therapy. One being an improved clinical response and the other being avoiding systemic side effects. Other possible theories for the placebo effect include, the application of oils to the skin. Although this may run counter to current thinking that oily material should be avoided, the presence of such materials may actually help improve the skin, and/or extract sebaceous matter from the gland or pores and/or have a negative feedback so as to reduce the production of material that can block or interfere with the operation of the sebaceous glands. Other contributing factors may include the presence of fatty alcohols; and/or the presence of a fatty acid; and/or the presence of waxes in the formulation.

The percentage reduction in inflammatory lesions and also in non-inflammatory lesions was found to be greater than seen with other current acne treatments. For example, application of 4% topical minocycline resulted in a 72% mean reduction of inflammatory lesions and 73% mean reduction of non-inflammatory lesions, respectively, at 12 weeks. In contrast, oral minocycline (Solodyn™) efficacy results presented in the Patient Package Insert show an overall reduction of ˜44% (43.1 study land 45.8 study 2) in inflammatory lesions and no effect on non-inflammatory lesions. So apart from the avoidance of unwanted systemic effects, topical minocycline treatment appears to have substantial advantages over oral minocycline treatment of acne. The improvement indicated in the Investigators Global Assessment (“IGA”) was also very encouraging. The percentage of patients, for example, with an IGA at 12 weeks of “almost clear” or “clear” was 20% for the placebo; 37% for minocycline (1%) and 53% for minocycline (4%). Even after treatment ceased, patients' IGA scores continued to improve. The patients' feedback was likewise positive. High overall patient satisfaction was reported with both 1% and 4% minocycline with more satisfaction, on average being seen with 4% minocycline. No drug related systemic side effects were observed. The product is considered safe and there were only two mild skin related adverse events reported.

The foamable composition in one embodiment is presented as a foam. In one or more embodiments the foam is a breakable foam. In another embodiment it is presented as a gel. In some embodiments the gel is liquid, in other embodiments it is semi-solid and in still further embodiments it is stable e.g. such that if inverted it generally maintains its shape. In one or more embodiments when a mechanical or shear force is applied to the gel it becomes flowable or liquid. Whilst the clinical trial was on the foam formulation, it is anticipated that the gel will have a similar or comparable therapeutic effects. In one or more embodiments the resultant foam has the same formulation as the gel after dissipation of propellant. In one or more embodiments the gel only differs from the foam by the absence of a propellant. Upon release of the composition (gel plus propellant) from the canister, the propellant dissipates and a foam is generated, having the composition of the gel but in the form of a foam. Furthermore, in certain cases, when the foam is collapsed it can revert to a formulation having gel-like properties.

In the course of treatment the compositions were able to reduce symptoms and severity of acne. Improvement was apparent as was the restoration of visible, normal cutaneous topographic features, indicating the return of skin integrity.

Remarkably, no serious adverse events were noted. Moreover, even skin irritation events associated with oral minocycline treatment route as well as other available topical formulations (for example erythema, peeling, itching and dryness) were almost not observed. Only a few mild cases of erythema, peeling and/or dryness were reported during treatment but they were transient and disappeared and there was no need to discontinue treatment. Topical foamable compositions comprising minocycline appear to avoid or minimize known side effects and may act to prevent or minimize such side effects, thereby leading to better patient compliance compared to available treatment options.

A Phase II, multi-center, randomized, placebo controlled and double blinded, parallel group, dose finding clinical study in acne patients has been completed. Safety and tolerability observations have revealed that these topical minocycline formulations are safe and well-tolerated. Likewise the placebo formulation without minocycline has also been found to be safe and well-tolerated.

It has been observed that many patients who completed between 6-12 weeks of treatment demonstrated a substantial decrease in the number of inflammatory and non-inflammatory lesions, which is greater than the decreases recorded for oral minocycline and for three recently approved topical products which use active ingredients other than tetracycline antibiotics, namely Epiduo™, Acanya™ and Ziana™. A decrease in the number of inflammatory and non-inflammatory lesions was demonstrated as early as three weeks from the begining of treatment.

It follows that foamable compositions comprising minocycline are safe, well-tolerated and can effectively treat acne vulgaris using a once daily topical application regime to an infected area while avoiding unwanted systemic side effects associated with oral treatment with antibiotics and skin irritation.

Thus, foamable compositions containing minocycline offer a safe, user friendly, and effective alternative to current oral minocycline treatments and other topical treatments.

In one or more embodiments there is provided a method of treating or alleviating a disorder selected from the group consisting of acne, acne related symptoms, a tetracycline antibiotic responsive acne related disorder, a tetracycline antibiotic responsive skin disorder, skin disorder caused by a bacteria, a sebaceous gland disorder, P. acne bacteria associated disorders and other superficial infections, including skin infections, comprising administering topically at least alternate days or once daily to a target area on a human subject having the disorder a hydrophobic gel or foam composition comprising a tetracycline antibiotic wherein the target area is the skin.

In one or more embodiments there is provided a method of treating or alleviating acne. In one or more embodiments there is provided a method of treating or alleviating acne related symptoms. In one or more embodiments there is provided a method of reducing the number of inflammatory lesions in a patient diagnosed with acne. In one or more embodiments there is provided a method of reducing the number of non-inflammatory lesions in a patient diagnosed with acne. In one or more embodiments there is provided a method of improving the success rate in treatment of a patient diagnosed with acne as measured by the mean percentage change of lesions. In one or more embodiments there is provided a method of improving the success rate in treatment of a patient diagnosed with acne as measured by improving the percentage of patients with an IGA of “almost clear” or “clear”. In one or more embodiments there is provided a method of treating or alleviating a tetracycline antibiotic responsive acne related disorder. In one or more embodiments there is provided a method of treating or alleviating a tetracycline antibiotic responsive skin disorder. In one or more embodiments there is provided a method of treating or alleviating skin disorder caused by a bacteria. In one or more embodiments there is provided a method of treating or alleviating a tetracycline antibiotic responsive disorder. In one or more embodiments there is provided a method of treating or alleviating a sebaceous gland disorder. In one or more embodiments there is provided a method of treating or alleviating superficial infections, including skin infections. In one or more embodiments there is provided a method of preventing the reoccurrence of acne. In one or more embodiments there is provided a method of preventing the reoccurrence of acne related symptoms. In one or more embodiments there is provided a method of preventing the reoccurrence of a tetracycline antibiotic responsive acne related disorder. In one or more embodiments there is provided a method of preventing the reoccurrence of a tetracycline antibiotic responsive skin disorder. In one or more embodiments there is provided a method of preventing the reoccurrence of skin disorder caused by a bacteria. In one or more embodiments there is provided a method of preventing the reoccurrence of a tetracycline antibiotic responsive disorder. In one or more embodiments there is provided a method of preventing the reoccurrence of a sebaceous gland disorder. In one or more embodiments there is provided a method of preventing the reoccurrence of superficial infections, including skin infections. In any one or more embodiments any of the above methods comprises applying a composition comprising a carrier and a tetracycline antibiotic topically to a target area of skin on a subject diagnosed with acne. In one or more embodiments, the composition is an oil based carrier and a tetracycline antibiotic. In one or more embodiments the composition and carrier is surfactant free. In one or more embodiments the composition and the carrier is polymeric agent free. In one or more embodiments, the composition and the carrier are ethanol free. In one or more embodiments the composition and the carrier is polyol free. In one or more embodiments, the composition and the carrier is hydrophilic solvent free. In one or more embodiments, the carrier comprises a hydrophobic solvent and a fatty alcohol. In one or more embodiments, the carrier comprises a hydrophobic solvent and a fatty alcohol and a wax. In one or more embodiments, the carrier comprises a hydrophobic solvent and a fatty alcohol and a wax and a fatty acid. In one or more embodiments, the composition is a gel composition and/or the carrier is a gel. In one or more embodiments the composition is a foamable composition and/or the carrier is a foamable composition.

Topical administration of minocycline was found to be effective in the treatment of inflammatory lesions. Minocycline inter alia decreases the production of substances that can cause inflammation and therefore has anti-inflammatory effects in general, mitigates apoptosis, and is thought to reduce pro-inflammatory cytokines. In one or more embodiments there is provided a topical gel or foam comprising minocycline to treat conditions, in which inflammation, and/or apoptosis and/or pro-inflammatory cytokines are part of their etiological factors. Non-limiting examples of such disorders of the skin include dermatitis, atopic dermatitis, contact dermatitis, perioral dermatitis, stasis dermatitis, seborrheic dermatitis, rosacea, psoriasis, rash, diaper rash, light-induced burn, sun burn, chemical burn, radiation burn.

Thus, in one or more embodiments there is provided a method of treating or alleviating inflammation by applying a minocycline gel or foam topically to an area of inflamed skin or mucosa on a subject in need of treatment.

In one or more embodiments there is provided a method of treating or alleviating conditions, in which inflammation, pro-inflammatory cytokines and/or apoptosis is a part of their etiological factors.

In one or more embodiments there is provided a method of treating or alleviating skin inflammation.

In one or more embodiments there is provided a method of treating or alleviating a disorder, selected from the group consisting of a dermatitis, atopic dermatitis, contact dermatitis, perioral dermatitis, stasis dermatitis, seborrheic dermatitis, rosacea, psoriasis, rash, diaper rash, light-induced burn, sun burn, chemical burn, radiation burn.

In one or more embodiments there is provided a method of treating or alleviating acne or acne related symptoms by applying a minocycline gel or foam topically to an area of inflamed skin or mucosa on a subject in need of treatment. In one or more embodiments, the hydrophobic gel or foam composition used in the method comprises:
a) about 60% to about 99% by weight of at least one hydrophobic solvent;
b) at least one viscosity-modifying agent selected from the group consisting of a fatty alcohol, a fatty acid, and a wax; and
c) a therapeutically effective amount of a tetracycline antibiotic.

In one or more embodiments the viscosity modifying agent comprises a wax in combination with a fatty alcohol or a fatty acid or both. In one or more embodiments the viscosity modifying agent comprises mixtures of fatty alcohols and a wax. In one or more embodiments the viscosity modifying agent comprises mixtures of fatty alcohols and waxes. In one or more embodiments the viscosity modifying agent comprises mixtures of fatty acids and a wax. In one or more embodiments the viscosity modifying agent comprises mixtures of fatty acids and waxes. In one or more embodiments the wax comprises a hydrogenated oil. In one or more embodiments the hydrogenated oil is a hydrogenated castor oil. In one or more embodiments the wax comprises a paraffin wax. In one or more embodiments the wax comprises a beeswax.

In one or more embodiments the method comprises:
a) expelling a tetracycline foamable formulation from a canister;
b) applying the resultant foam to an area of skin or mucosa;
c) collapsing the foam.

In one or more embodiments the method comprises:
a) expelling a tetracycline foamable formulation from a canister;
b) applying the resultant foam to an area of skin or mucosa;
c) spreading the foam.

In one or more embodiments the spread or collapsed foam is absorbed into the skin or mucosa. In one or more embodiments the absorption is rapid. In one or more embodiments it is mostly absorbed or within about 30 seconds, or within about 20 seconds, or within about 15 seconds, or within about 10 seconds, or within about 5 seconds, or within about 3 seconds or within about a second. In one or more embodiments absorption is within about 30 seconds, or within about 20 seconds, or within about 15 seconds or within about 10 seconds, or within about 5 seconds, or within about 3 seconds or within about a second.

In one or more embodiments the hydrophobic foam used in the method is formed from the hydrophobic gel composition further comprising a propellant.

In an embodiment the disorder is acne vulgaris.

In an embodiment the disorder is an inflammatory disorder.

In an embodiment the disorder is a skin or mucosal related inflammatory disorder or a skin or mucosal related inflammation, or a condition, in which inflammation, pro-inflammatory cytokines and/or apoptosis is part of its etiological factors.

In an embodiment the disorder is a non-inflammatory disorder.

In an embodiment the disorder displays one or more lesions selected form the group consisting of comedonal, papulopustular, nodulocystic, and mixtures of any two or more thereof.

In one or more embodiments the tetracycline antibiotic used in the method is selected from the group consisting of tetracycline, oxytetracycline, demeclocycline, doxycycline hyclate, lymecycline, meclocycline, methacycline, minocycline hydrochloride, rolitetracycline, chlorotetracycline, and tigecycline.

In one or more embodiments the tetracycline antibiotic used in the method is present in the composition at a concentration of about 0.5% to about 10% by weight.

In one or more embodiments the tetracycline antibiotic used in the method is minocycline hydrochloride. In one or more embodiments the minocycline hydrochloride used in the method is present in the composition at a concentration of about 1% by weight. In one or more embodiments the minocycline hydrochloride used in the method is present in the composition at a concentration of about 4% by weight.

In one or more embodiments the hydrophobic gel or foam composition used in the method is applied on average at a frequency selected from the group consisting of three times daily, twice daily, once daily, and alternate day.

In one or more embodiments, the hydrophobic gel or foam composition used in the method is administered for a period selected from the group consisting of two weeks, three weeks, four weeks, five weeks, six weeks, seven weeks, eight weeks, nine weeks, ten weeks, eleven weeks, twelve weeks, thirteen weeks, fourteen weeks, fifteen weeks, and sixteen weeks. In one or embodiments, the hydrophobic gel or foam composition used in the method is administered for a period longer than 16 weeks, for example seventeen or eighteen or nineteen, twenty, twenty-one, twenty-two, twenty-three or twenty-four weeks or such longer period as is needed to bring the disorder under control.

In one or more embodiments, the hydrophobic gel or foam composition used in the method is applied as a maintenance dose after the therapy period at a frequency selected from the group consisting of every two days, three times a week, twice a week, once a week, once in two weeks, once in three weeks, once a month, once in two months, and alternate weeks. In one or more embodiments, the maintenance dose is discontinued after a period selected from the group consisting of a week, two weeks, three weeks, four weeks, a month, two months, three months, four months, five months, and six months.

In one or more embodiments the hydrophobic foam composition or gel used in the method is effective against P. acne bacteria associated disorders.

In one or more embodiments the hydrophobic foam composition or gel used in the method results in a decrease of at least about 60% in the number of the acne vulgaris inflammatory lesions after twelve weeks of treatment, wherein the hydrophobic foam composition or gel is administered once daily. In one or more embodiments the decrease is at least about 65%, or at least about 70%.

In one or more embodiments the hydrophobic foam composition or gel used in the method results in a decrease of at least about 55% in the number of the acne vulgaris inflammatory lesions after nine weeks of treatment, wherein the hydrophobic foam composition or gel is administered once daily. In one or more embodiments the decrease is at least about 60%, or at least about 65%, or at least about 70%.

In one or more embodiments the hydrophobic foam composition or gel used in the method results in a decrease of at least about 50% in the number of the acne vulgaris inflammatory lesions after six weeks of treatment, wherein the hydrophobic foam composition or gel is administered once daily. In one or more embodiments the decrease is at least about 55%, or at least about 60%, or at least about 65%, or at least about 70%.

In one or more embodiments the hydrophobic foam composition or gel used in the method results in a decrease of at least about 45% in the mean number of the acne vulgaris inflammatory lesions after three weeks of treatment, wherein the hydrophobic foam composition or gel is administered once daily. In one or more embodiments the decrease is at least about 50%, or at least about 55%, or at least about 60%. In an embodiment the decrease can be at least about 40%.

In one or more embodiments the hydrophobic foam composition or gel used in the method results in a decrease of at least about 50% in the number of the acne vulgaris inflammatory lesions four weeks after the end of the treatment. In one or more embodiments the decrease is at least about 55%, or at least about 60%, or at least about 65%, or at least about 70%.

In one or more embodiments the hydrophobic foam composition or gel used in the method obtains a decrease of at least about 55% in the number of the acne vulgaris non-inflammatory lesions after twelve weeks of treatment, wherein the composition is administered once daily. In one or more embodiments the decrease is at least 60%, or at least about 65%, or at least about 70% or at least about 75%.

In one or more embodiments the hydrophobic foam composition or gel used in the method results in a decrease of at least about 50% in the number of the acne vulgaris non-inflammatory lesions after nine weeks of treatment, wherein the hydrophobic foam composition or gel is administered once daily. In one or more embodiments the decrease is at least about 55%, or at least about 60%, or at least about 65%, or at least about 70%.

In one or more embodiments the hydrophobic foam composition or gel used in the method results in a decrease of at least about 45% in the number of the acne vulgaris non-inflammatory lesions after six weeks of treatment, wherein the hydrophobic foam composition or gel is administered once daily. In one or more embodiments the decrease is at least about 50%, or at least about 55%, or at least about 60%, or at least about 65%, or at least about 70%.

In one or more embodiments the hydrophobic foam composition or gel used in the method results in a decrease of at least about 40% in the number of the acne vulgaris non-inflammatory lesions after three weeks of treatment, wherein the hydrophobic foam composition or gel is administered once daily. In one or more embodiments the decrease is at least about 45%, or at least about 50%, or at least about 55%, or at least about 60%.

In one or more embodiments the hydrophobic foam composition or gel used in the method obtains a decrease of at least about 60% in the number of the acne vulgaris non-inflammatory lesions four weeks after the end of the treatment.

In one or more embodiments the hydrophobic gel or foam composition used in the method comprises:
about 48% to about 51% by weight of soybean oil;
about 23% to about 25% by weight of coconut oil;
about 4% to about 6% by weight of cyclomethicone;
about 3% to about 5% by weight of light mineral oil;
about 3% to about 4% by weight of cetostearyl alcohol;
about 2% to about 4% by weight of stearic acid;
about 2% to about 3% by weight of myristyl alcohol;
about 1% to about 3% by weight of hydrogenated castor oil;
about 1% to about 3% by weight of beeswax;
about 1% to about 2% by weight of stearyl alcohol;
about 0.5% to about 1.5% by weight of behenyl alcohol;
about 0.2% to about 0.5% by weight of modified (fumed) silica; and
about 1% by weight of minocycline hydrochloride or doxycycline hyclate.

In one or more embodiments the hydrophobic gel or foam composition used in the method comprises:
about 48% to about 51% by weight of soybean oil;
about 23% to about 25% by weight of coconut oil;
about 4% to about 6% by weight of cyclomethicone;
about 0.5% to about 1.5% by weight of light mineral oil;
about 3% to about 4% by weight of cetostearyl alcohol;
about 2% to about 4% by weight of stearic acid;
about 2% to about 3% by weight of myristyl alcohol;
about 1% to about 3% by weight of hydrogenated castor oil;
about 1% to about 3% by weight of beeswax;
about 1% to about 2% by weight of stearyl alcohol;
about 0.5% to about 1.5% by weight of behenyl alcohol;
about 0.2% to about 0.5% by weight of modified (fumed) silica; and
about 4% by weight of minocycline hydrochloride or doxycycline hyclate.

In one or more embodiments if the tetracycline antibiotic concentration is increased one or more of the oils is decreased by a corresponding amount. Likewise in one or more embodiments if the tetracycline antibiotic concentration is decreased one or more of the oils is increased by a corresponding amount.

In one or more embodiments the hydrophobic foam used in the method is formed from the hydrophobic gel composition further comprises about 3% to about 25% by weight of propellant based on the total weight of the hydrophobic gel composition.

In one or more embodiments the hydrophobic foam composition or gel used in the method obtains a decrease of at least about 45% in the total number of the acne vulgaris lesions (inflammatory plus non-inflammatory lesions) after twelve weeks or less than twelve weeks of treatment, wherein the composition is administered once daily. In one or more embodiments the decrease is at least about 50%, or at least about 55%, or at least about 60%, or at least about 65% or at least about 70%. In an embodiment the decrease can be at least about 40%.

In one or more embodiments the hydrophobic gel or foam composition used in the method obtains a decrease in the total number of lesions after nine weeks or less than nine weeks of treatment, when the composition is administered on average once daily. In an embodiment the decrease can be at least about 50%. In one or more embodiments the decrease is at least about 55%, or at least about 60%, or at least about 62%, or at least about 65%, or at least about 68%, or at least about 70%.

In one or more embodiments the hydrophobic gel or foam composition used in the method obtains a decrease in the total number of lesions after six weeks or less than six weeks of treatment, when the composition is administered on average once daily. In an embodiment the decrease can be at least about 45%. In one or more embodiments the decrease is at least about 50%, or at least about 55%, or at least about 60%, or at least about 62%, or at least about 65%, or at least about 68%, or at least about 70%.

In one or more embodiments the hydrophobic gel or foam composition used in the method obtains a decrease in the total number of lesions after three weeks or less than three weeks of treatment, when the composition is administered on average once daily. In an embodiment the decrease can be at least about 40%. In one or more embodiments the decrease is at least about 42%, or at least about 45%, or at least 47%, or at least 50%, or at least about 55%, or at least about 60%, or at least about 62%, or at least about 65%, or at least about 68%, or at least about 70%.

In one or more embodiments the hydrophobic foam composition or gel used in the method obtains a decrease of at least 55% in the total number of the acne vulgaris lesions when observed four weeks after the end of the treatment. In one or more embodiments the decrease is at least about 57%, or at least about 60%, or at least about 62%, or at least about 65%, or at least about 68%, or at least about 70%.

In one or more embodiments the hydrophobic foam composition or gel used in the method is safe and tolerated when the hydrophobic gel or foam composition is administered once daily for a period of at least six weeks.

In one or more embodiments the tolerability of the hydrophobic foam composition or gel used in the method is determined by skin irritation and wherein symptoms for assessing skin irritation are selected from a group consisting of pigmentation, erythema, dryness, peeling, and itching.

In one or more embodiments the hydrophobic foam composition or gel used in the method is safe, and tolerated and has high rates of clinical responses when the hydrophobic gel or foam composition is administered once daily for at least two weeks.

In one or more embodiments the method comprises a step of administering which includes releasing the hydrophobic gel or foam composition from a container and applying it onto the target area by collapsing and/or spreading it on the target area using mild mechanical force, such as a simple rub, thereby resulting in the hydrophobic gel or foam composition collapsing and being absorbed onto the target area. In one or more embodiments method further comprises using a sterile applicator or prior to the steps of administering and/or collapsing and/or spreading, the hands of the person spreading are sterilized in order to avoid cross contamination.

In one or more embodiments the hydrophobic gel or foam composition used in the method is absorbed within at least 120 seconds.

In one or more embodiments the hydrophobic gel or foam composition used in the method obtains a decrease in the number of lesions after twelve weeks or less than twelve weeks of treatment, when the composition is administered on average once daily.

In one or more other embodiments the composition is administered twice daily. In one or more further embodiments the composition is administered thrice daily.

In an embodiment the acne is severe prior to starting treatment. In an embodiment the acne is of moderate severity prior to starting treatment. In an embodiment the acne is of mild severity prior to starting treatment. In an embodiment the acne is almost clear prior to starting treatment. In one or more embodiments the treatment reduces the acne from severe to moderate severity or to mild severity or to almost clear or to clear. In one or more embodiments the treatment reduces the acne from moderate severity to mild severity or to almost clear or to clear. In one or more embodiments the treatment reduces the acne from mild severity to almost clear or to clear. In an embodiment the treatment reduces the acne from almost clear to clear.

In an embodiment the subject is under the age of 25 years old. In one embodiment the subject is under the age of thirty years old.

In one embodiment the subject the subject is under the age of forty-six years old and is a pregnant or breastfeeding female.

In one or more embodiments the hydrophobic gel or foam composition used in the method has a shelf life of at least two years at ambient temperature.

In one or more embodiments there is provided a method for retarding, arresting, or reversing the progression of a disorder in a mammalian subject in need thereof, the disorder selected from the group consisting of acne, acne related symptoms, a tetracycline antibiotic responsive acne related disorder, a tetracycline antibiotic responsive skin disorder, skin disorder caused by a bacteria, a tetracycline antibiotic responsive disorder, a sebaceous gland disorder, P. acne bacteria associated disorders and other superficial infections, including skin infections, the method comprising topically applying to the skin of the subject a hydrophobic foam composition or gel comprising a tetracycline antibiotic at least alternate days or once a day for at least six weeks, thereby retarding, arresting, or reversing the progression of the disorder in the subject.

In one or more embodiments there is provided a method for retarding, arresting, or reversing the progression of a disorder wherein the hydrophobic gel or foam composition comprises:
about 60% to about 99% by weight of at least one hydrophobic solvent;
at least one viscosity-modifying agent selected from the group consisting of a fatty alcohol, a fatty acid, and a wax; and
a therapeutically effective amount of a tetracycline antibiotic.

In one or more embodiments there is provided a method for retarding, arresting, or reversing the progression of a disorder wherein the tetracycline antibiotic is selected from the group consisting of tetracycline, oxytetracycline, demeclocycline, doxycycline hyclate, lymecycline, meclocycline, methacycline, minocycline, minocycline hydrochloride, rolitetracycline, chlorotetracycline, and tigecycline.

In one or more embodiments there is provided a method for retarding, arresting, or reversing the progression of a disorder wherein the tetracycline antibiotic is minocycline hydrochloride.

In one or more embodiments there is provided a method for retarding, arresting, or reversing the progression of a disorder wherein the tetracycline antibiotic is minocycline hydrochloride at a concentration of about 1% or about 4% by weight.

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

### BRIEF DESCRIPTION OF THE DRAWINGS

The invention is described with reference to the drawings, which are presented for the purpose of illustration only and is not intended to be limiting of the invention. In one or more embodiments:

FIG. 1 is a pictorial example of acne vulgaris lesions on the forehead of a fourteen year old male observed before and after receiving treatment with a 1% minocycline foam once daily over the course of six weeks.

FIG. 2 is a pictorial example of acne vulgaris lesions on the forehead of a seventeen year old male observed before and after receiving 4% minocycline for 12 weeks.

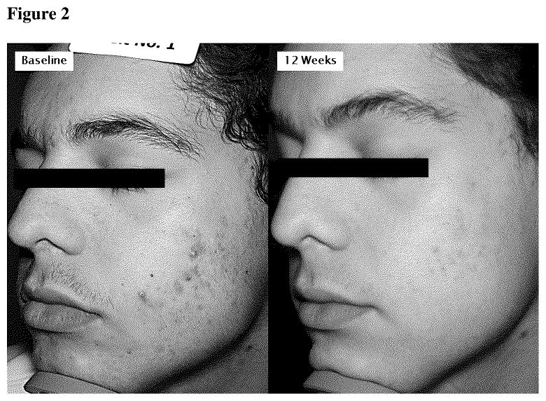

FIG. 3 is a pictorial example of histological sections of minocycline foam penetrating hair follicles using fluorescence microscopy, reflectance and fluorescent images being superimposed.

FIG. 4 provides a chart summarizing the mean inflammatory lesion count in the ITT population by visit and by study group.

FIG. 5 provides a chart summarizing the mean reduction in the number of inflammatory lesion in the ITT population by visit and by study group.

FIG. 6 provides a chart summarizing the mean percent reduction of the number of inflammatory lesions in the ITT population by visit and by study group.

FIG. 7 provides a chart summarizing the percent of subjects who had a decrease of more than 60%, in inflammatory lesions count in the ITT population by visit and by study group.

FIG. 8 provides a chart summarizing the mean percent reduction of the number of non-inflammatory lesions in the ITT population by visit and by study group.

FIG. 9 provides a chart summarizing the mean percent reduction of the total number of lesions in the ITT population by visit and by study group.

FIG. 10 provides a chart summarizing the mean IGA score in the ITT population by visit and by study group.

FIG. 11 provides a chart summarizing the percent of subjects with mean IGA score less than 2 in the ITT population by visit and by study group.

### DETAILED DESCRIPTION

In one or more embodiments there is provided herein, a method for treating a disorder of the skin or a mucosal surface, especially when the disorder is a disorder of a sebaceous gland. In one or more embodiments there is provided herein, a method for treating a disorder including acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infection, including skin infections. In one or more embodiments the disorder involves inflammation. The method includes administering topically to a surface having the disorder a therapeutic hydrophobic composition, comprising a tetracycline antibiotic. In one or more embodiments the hydrophobic composition comprises a carrier comprising about 60% to about 99% by weight of at least one hydrophobic solvent; at least one viscosity-modifying agent selected from the group consisting of a fatty alcohol, a fatty acid and a wax; and a tetracycline antibiotic.

According to one or more embodiments provided herein the tetracycline is a minocycline or doxycycline, which are semi-synthetic tetracycline antibiotic. According to one or more embodiments, the tetracycline is minocycline. The tetracycline drug is usually bacteriostatic in action. It can, amongst other options, exert its antimicrobial activity by inhibiting protein synthesis. It can also have an antiviral effect. According to one or more embodiments the minocycline is minocycline hydrochloride (minocycline HCl; (hereinafter “MCH”)). MCH is a yellow crystalline powder that is sparingly soluble in water, slightly soluble in alcohol and practically insoluble in chloroform and in ether.

Minocycline is known to be highly sensitive to air and light and undergoes rapid degradation. Therefore storage of foamable formulations in airtight sealed containers under pressure with propellant may contribute to preserving stability subject to selection of compatible canisters and accessories. Likewise, production and/or filing under vacuum in an oxygen free environment can help.

The ingredients of the carrier were selected for their compatibility with tetracycline antibiotics as described. In one or more embodiments it was not sufficient to identify single ingredients that were compatible with tetracycline antibiotics but formulations had to be found in which the ingredients in combination were also compatible with tetracycline antibiotics.

In one or more embodiments, a hydrophobic foamable composition (e.g. foam or gel) provided herein comprises:
a) about 60% to about 99% by weight of at least one hydrophobic solvent;
b) about 1% to about 22% by weight of at least one viscosity modifying agent; and
c) about 0.1% to about 18% of a tetracycline antibiotic (e.g., minocycline HCl).

In one or more embodiments, a hydrophobic foamable composition or gel provided herein comprises:
a) about 70% to about 90% by weight of at least one hydrophobic solvent;
b) about 10 to about 22% by weight of at least one viscosity modifying agent; and
c) about 0.5% to about 8% of a tetracycline antibiotic (e.g., minocycline HCl).

In one or more embodiments, a hydrophobic foamable composition or gel provided herein comprises:
a) about 75% to about 90% by weight of at least one hydrophobic solvent;
b) about 10 to about 22% by weight of at least one viscosity modifying agent; and
c) about 0.5% to about 2% of a tetracycline antibiotic (e.g., minocycline HCl).

In one or more embodiments, a hydrophobic foamable composition or gel provided herein comprises:
a) about 72% to about 88% by weight of at least one hydrophobic solvent;
b) about 10 to about 22% by weight of at least one viscosity modifying agent; and
c) about 2% to about 6% of a tetracycline antibiotic (e.g., minocycline HCl).

According to one or more embodiments there are provided substantially surfactant-free oleaginous formulations comprising a tetracycline, such as a minocycline, for use in treatment of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections. In one or more embodiments the tetracycline is used for the treatment of rosacea. In one or more embodiments the tetracycline is used for the treatment of impetigo. In one or more embodiments the tetracycline acts to reduce oxidative stress and/or inflammation in skin pathologies. In one or more embodiments the tetracycline is effective where the condition is accompanied by apoptotic cell death.

### Definitions

All % values are provided on a weight (w/w) basis.

By the term “about” herein it is meant that a figure or range of figures can vary plus or minus up to 10%. So in this embodiment if a figure of “about 1” is provided then the amount can be up to 1.1 or from 0.9. As will be appreciated by one of the art there is some reasonable flexibility in formulating compositions such that where one or more ingredients are varied successful formulations may still be made even if an amount falls slightly outside the range. Therefore, to allow for this possibility amounts are qualified by about. In one or more other embodiments the figures may be read without the prefix about.

The term “thixotropic,” as used herein, means that the formulation shows a decrease in viscosity upon application of shear force. The structure of the formulation breaks down, leading to a reduction in viscosity. When the formulation is standing without shear force, this decrease in viscosity is recovered over time.

It should be noted that the term “gel” means a jelly-like material that can have properties ranging from soft and fluid to hard and tough. Gels may be in liquid, semi-liquid, semi-solid or solid state. Solid gels are defined as a substantially diluted crosslinked system, which exhibits no flow when in the steady-state. By weight, gels are mostly liquid, yet they behave like semi-solids due to a three-dimensional crosslinked network of a solidifying, gelling or thickening agent within the liquid. It is the crosslinks within the fluid that give a gel its structure (hardness) and contribute to stickiness (tack). Depending on the amounts of gelling agent in a formulation, the gel may be semi-solid with some limited flowability, such that when the semi-solid gel is placed in a tube and is inclined horizontally from a vertical position it will slowly flow from the vertical towards the horizontal or it may be a liquid gel where the amount of gelling agent or gelling effect is lower such that the gel structure or connections are weaker or loose so that when placed in a tube and tilted from a vertical position to the horizontal the gel readily flows and adapts to the horizontal position. The rheological properties of gels at different surface temperatures can influence the release and bioabsorption of drugs therefrom.

In some embodiments, formulations comprising a hydrophobic oil and viscosity-modifying agents demonstrated increased viscosity of such oil, and to which when even small amounts of a suspended tetracycline antibiotic were added, a substantial or synergistic increase in the viscosity of the composition was observed.

In one or more embodiments, the gel is stable and it retains its viscosity upon dispensing from a container, such as a tube, yet, it liquefies and spreads easily upon application of shear force, which can be mild, such as a simple rub. Further, while the gel is oily, it absorbs into the site of application, such as the skin or mucosa membrane, and after minutes the surface does not appear and/or feel significantly oily or greasy.

The term “liquid gel” refers inter alia to the formulation after propellant is added (which prior to adding the propellant is a gel) or where the gel is loose or fluid or such that when subjected to gravity will pour or become liquid.

The term “waterless” or “water free” as used herein, means that the composition contains no, or essentially no, free or unassociated or absorbed water. Similarly, “substantially water free” or “substantially waterless” carriers contain at most incidental or trace amounts of water. In one or more embodiments, “substantially waterless” or substantially water free” means the composition contains about or less than 1%, (e.g. about or less than 0.8%; about or less than 0.6%; about or less than 0.4%; about or less than 0.2%; about or less than 0.1%, about or less than 0.05%, about or less than 0.01%, less than about 0.001% by weight, or 0% by weight).

By the term “single phase” herein it is meant that after addition of propellant to the composition or carrier, the liquid components of the foamable composition or carrier are fully miscible, and the solid components, if any, are either dissolved or homogeneously suspended in the composition so that only one phase is visible.

By the term “substantially a single phase” is meant that the composition or carrier after addition of propellant is primarily or essentially a single phase as explained above, but may also have present a small amount of material which is capable of forming or may form a separate phase amounting to less than about 5% of the composition or carrier after the addition of propellant. In some embodiments, it is less than about 3%, and other such embodiments, is less than about 1%.

The term “unstable active agent” as used herein, means an active agent which is oxidized and/or degraded within less than a day, and in some cases, in less than an hour upon exposure to air, light, skin, or water or a pharmaceutical excipient under ambient conditions.

It should be noted that the term “surfactant” or “emulsifier” in the context herein refers to stand alone surfactants used to reduce surface tension between two substances or phases, which are also capable of stabilizing an emulsion of water and oil. Reduction of surface tension can be significant in foam technology in relation to the ability to create small stable bubbles. This is as opposed to the term surfactant which has often been loosely used in the art to include substances which do not function effectively as standalone surfactants to reduce surface tension between two substances or phases and which are also capable of stabilizing an emulsion of water and oil. For example, a surfactant as provided herein, does not include fatty acids, does not include fatty alcohols and does not include propoxylated lanolin oil derivatives. In the context of the present invention fatty acids and fatty alcohols are defined as foam adjuvants. Similarly, propoxylated lanolin oil derivatives in the context herein are defined as emollients.

“Standard surfactant” or “customary surfactant” or “stand alone surfactant” refers to customary non-ionic, anionic, cationic, zwitterionic, amphoteric and amphiphilic surfactants. Many standard surfactants are derivatives of fatty alcohols or fatty acids, such as ethers or esters formed from such fatty alcohols or fatty acids with hydrophilic moieties, such as polyethylene glycol (PEG). However, a native (non-derivatized) fatty alcohol or fatty acid, as well as waxes are not regarded as a standard surfactant.

The term “co-surfactant” as used herein, means a molecule which on its own is not able to form and stabilize satisfactorily an oil-in-water emulsion but when used in combination with a surfactant as defined herein the co-surfactant has properties which can allow it to help a surfactant create an emulsion and can boost the stabilizing power or effect of the surfactant. Examples include a fatty alcohol, such as cetyl alcohol, or a fatty acid, such as stearic acid. Cetyl alcohol is a waxy hydrophobic substance that can be emulsified with water using a surfactant. Some substances may have more than one function and for example, fatty alcohols can in some formulations act as a co-solvent. In certain circumstances, a co-surfactant can itself be converted into a surfactant or soap by, for example, adding a base, such as, triethanolamine to a fatty acid like stearic acid.

The term “viscosity modifying agent” in the context of the present disclosure is an agent which, when added to a hydrophobic oil, facilitates the creation of a hydrophobic breakable vehicle in the form of a breakable gel or breakable foam. In one or more embodiments, the viscosity modifying agent is a “foamer complex” comprising a fatty alcohol, a fatty acid and/or a wax.

The term “breakable” refers to a unique property of the gel or the foam wherein the gel or foam is stable upon dispensing from a container, yet breaks and spreads easily upon application of shear or mechanical force, which can be mild such as a simple rub.

It should be noted that the term a “polyol”, as used herein, is an organic substance that contains at least two hydroxy groups in its molecular structure.

The term “water activity” as used herein, represents the hygroscopic nature of a substance, or the tendency of a substance to absorb water from its surroundings. Microorganisms require water to grow and reproduce, and such water requirements are best defined in terms of water activity of the substrate. The water activity of a solution is expressed as Aw=P/Po, where P is the water vapor pressure of the solution and Po is the vapor pressure of pure water at the same temperature. Every microorganism has a limiting Aw, below which it will not grow; e.g., for Streptococci, Klebsiella spp, Escherichia coli, Clostridium perfringens, and Pseudomonas spp, the Aw value is 0.95. Staphylococcus aureus is most resistant and can proliferate with an Aw as low as 0.86, and fungi can survive at an Aw of at least 0.7. In one or more embodiments, the concentration of the hydrophobic solvent, and/or second rheology modulator in the composition is selected to provide an Aw value selected from the ranges between or of (1) about 0.8 and about 0.9; (2) about 0.7 and about 0.8; and (3) less than about 0.7. Delivering the formulation in a pressurized package does not allow for humidity to be absorbed by the preparation, and therefore, the water free character of the composition is not altered or compromised.

In one embodiment, no preservative is needed because the formulation is a waterless hydrophobic solvent or oil-based formulation having an Aw (water activity) value of less than 0.9, or less than about 0.8, or less than about 0.7, or less than about 0.6, and preferably less than about 0.5 which is below the level of microbial proliferation.

The identification of a “solvent,” as used herein, is not intended to characterize the solubilization capabilities of the solvent for any specific active agent or any other component of a gel or a foamable composition. Rather, such information is provided to aid in the identification of materials suitable for use as a part in a gel or a foamable composition as described herein.

It should be noted that the term “a method of treating a disease or a disorder” as provided throughout the specification is interchangeable with the term “use of the composition as a medicament for treatment of a disease”. It should be noted the term a disease is used interchangeably with the term disorder.

It should be noted that the term “substantially free of” an ingredient as provided throughout the specification is intended to mean that the composition comprises less than about 0.5% by weight (e.g., less than about 0.2% by weight, less than about 0.1% by weight, less than about 0.05% by weight, less than about 0.01% by weight, less than about 0.001% by weight, or 0% by weight) of an ingredient.

The term “surfactant free” or emulsifier free” or “non-surfactant” composition means compositions which comprise no or negligible levels of surface active agents. Where a formulation includes insignificant or de minimis amounts of surface active agents it is considered to be essentially surfactant free.

The term “substantially surfactant-free” relates to a composition wherein the ratio between the viscosity-modifying agent and the surfactant is between 10:1 or 5:1; or between 20:1 and 10:1 or between 100:1 and 20:1. In additional embodiments, the term relates to a composition that contains a total of less than about 0.4% of a surfactant selected from the group consisting of customary non-ionic, anionic, cationic, zwitterionic, amphoteric and ampholytic surfactants.

Preferably, the composition comprises less than about 0.2% by weight of a standard surfactant or less than about 0.1%; or less than 0.05%.

By de minimis is meant so minor as to merit disregard.

The term “hydrophobic gel composition” or “hydrophobic foam composition” or “hydrophobic composition” is intended to mean that the composition has a low solubility in water. In an embodiment, 100 to 1000 parts of water are needed to dissolve or render miscible 1 part of composition. In an embodiment, 1000 to 10,000 parts of water are needed to dissolve or render miscible 1 part of composition. In an embodiment, more than 10,000 parts of water are needed to dissolve or render miscible 1 part of composition.

By “regular basis” is meant a repeated or repeatable interval of time which can be by way of illustration, a part of a day, daily, alternate daily, twice weekly, weekly, fortnightly, monthly or some other repeated or repeatable interval for an appropriate period of time wherein a dose is to be applied. In this connection the repeat applications will be according to the needs of the subject and the disease or disorder. In some circumstances as little as three repeat doses may be required in other cases, between 3 and 14, in other cases between 14 and 28, in other cases between 28 and 50, in other cases between 50 and 75, in other cases between 75 and 100 and in other cases such as where prolonged treatment or a long period of maintenance dosing is needed as many as one, two or three hundred or more repeat doses may be needed.

The term clinical response to treatment, (clinical success or clinical failure) in the context of acne treatment is derived from an efficacy evaluation endpoints. The term total lesion count relates to the sum of inflammatory lesions and non-inflammatory lesions.

The terms “high rates of clinical response” or “high efficacy” or “substantial decrease” in the context herein can relate to a reduction of about 45% or more in lesion count (inflammatory, non-inflammatory or total); or to where subjects met a success criterion of “clear” or “almost clear” or to an “improvement of 2 grades from the baseline”; or to where subjects receive an excellent score according to Investigator's Global Improvement Assessment; or to where patients receive excellent in Patient's Global Improvement Assessment; or wherein according to any of the aforementioned endpoints a statistically significant reduction or improvement is demonstrated as compared to placebo.

The term safe in the context herein means having no or essentially no adverse events or serious adverse event.

The term adverse event in the context of the clinical study means having any untoward sign (including an abnormal vital sign, physical exam finding), symptom, or disease temporally associated with the use of a medical product whether or not considered as related to the medical product. A new condition or the worsening of a pre-existing condition was considered as an adverse event. All abnormal findings or diagnostic procedures (vital signs, physical exam, pregnancy etc) considered to be clinically significant (CS) were recorded as adverse events.

The term serious adverse event in the context herein can relate to any adverse drug event (experience) occurring at any dose that results in any of the following outcomes: death, a life-threatening adverse drug experience, inpatient hospitalization or prolongation of existing hospitalization (for >24 hours, a persistent or significant incapacity or substantial disruption of the ability to conduct normal life functions, a congenital anomaly/birth defect, an important Medical Events (IME) that may not result in death, be life threatening, or require hospitalization may be considered a serious adverse drug experience when, based upon medical judgment, they may jeopardize the patient or subject and may require medical or surgical intervention to prevent one of the outcomes listed in this definition.

The term tolerable or enhanced tolerability in the context herein means having no or essentially no skin irritation symptoms such as pigmentation, erythema, dryness, peeling and itching or alternatively when such symptoms arise they are mild and disappear without interrupting treatment.

By “essentially no” in the context of tolerability includes insignificant or de minimis occurrences of skin irritation events manifested in symptoms such as pigmentation, erythema, dryness, peeling and itching or mild transient events connected with the application of topical tetracyclines or vehicle.

By “essentially no” in the context of safety includes insignificant or de minimis occurrences of systemic or serious adverse events connected with the application of topical tetracyclines or vehicle.

The clinical response was determined at each study visit inter alia by a lesion count (inflammatory/non inflammatory and total), by % change in lesion count, by Investigator global assessment, by improvement assessment (by the Investigator) and improvement assessment (by the patient). Photographs were also used to assess the clinical improvement.

The term clinical failure is defined as insufficient improvement or deterioration (i.e. an increase or no change in the number of lesions).

By “on average” with reference to dosage regimes it is intended to reflect and/or take into account human nature and that a subject may forget to apply a dose or not strictly adhere to the regime, such that even if a subject forgets a dose or does not strictly adhere to the regime it will still be considered as if the regime has been applied. For example, if a subject misses an occasional dose but does not make it up or alternatively if having missed a dose applies a compensatory dose on a different day it is still counted as having complied with the dosage regime

An acne related disorder is any disorder which may occur in parallel with acne or be a contributing factor to the outbreak of acne or may resemble acne. Perioral dermatitis is an erythematous, papulopustular facial eruption that resembles acne and/or rosacea but typically starts around the mouth. Rosacea (acne rosacea) is a chronic inflammatory disorder characterized by facial flushing, telangiectasias, erythema, papules, pustules, and in severe cases, rhinophyma.

Acne related symptoms include, papules, pustules, blackheads, whiteheads or milia, nodules and cysts.

Acne conglobata is the most severe form of acne vulgaris, affecting men more than women. Patients have abscesses, draining sinuses, fistulated comedones, and keloidal and atrophic scars. Acne fulminans is acute, febrile, ulcerative acne, characterized by the sudden appearance of confluent abscesses leading to hemorrhagic necrosis. Leukocytosis and joint pain and swelling may also be present. Pyoderma faciale (also called rosacea fulminans) occurs suddenly on the midface of young women. It may be analogous to acne fulminans. The eruption consists of erythematous plaques and pustules.

In non-inflammatory acne, symptoms include microcomedones, blackheads, milia and closed comedones, bumps or bumpiness across the skin's surface, or an uneven skin texture, rough skin feel, non-inflamed acne blemishes, acne cosmetica, smoker's acne. Inflamed acne is characterized by redness and inflammation. Those with inflamed acne will have microcomedones, blackheads, and milia, as well as papules, pustules, and possibly nodules and cysts. Symptoms also include redness, swelling, and irritation of the skin, along with possible crusting, oozing, or scabbing of the lesions. Inflamed acne ranges in acuity from very mild to extremely severe and from only the occasional pustule to angry-looking cysts, skin damage and scarring and pigmentation. These symptoms can typically be seen on the face, neck, chest, shoulders, upper arms, back, and less commonly on the torso, arms, and legs. Discolored, darkened, or reddened spots or blotches are common on acne prone skin. These problems can persist even after breakouts have fully healed. Post-inflammatory hyperpigmentation (PIH) describes discolored spots (macules) left behind after an acne lesion has healed. Scars include ice pick scars, rolling scars, atrophic (depressed or pitted) scars, boxcar scars, hypertrophic (raised) scars and keloids.

In one or more embodiments, topical tetracycline antibiotics may be effective in treatment of one or more of bacterial infections, cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma, disorders of hair follicles and sebaceous glands, acne, rosacea, perioral dermatitis, hypertrichosis (hirsutism), alopecia, including male pattern baldness, alopecia greata, alopecia universalis and alopecia totalis, pseudofolliculitis barbae, and keratinous cyst. For example, rosacea involves papules and pustules, which can be treated with an antibiotic agent, as well as erythema, telangiectasia, and redness, which partially respond to treatment with an antibiotic agent.

In one or embodiments, topical tetracycline treatments may be given with or followed by application of a steroid or a hyaluronic acid or a collagen or a silicone or mixtures of any two or more thereof, for example to ameliorate or reduce scarring or skin damage effects.

It should be noted that hydrophobic compositions disclosed herein can be applied to the target site as a gel or a semi-solid gel or foam. In certain other embodiments, it can be applied as a liquid gel or as a collapsed foam. In one or more embodiments, the composition is thixotropic. In one or more embodiments, the gel formulation subjected to constant shear rate shows a reduction in viscosity with time. In one or more further embodiments, after the material is allowed to rest for a period of time, the viscosity increases again. In one or more embodiments, there is provided prior to adding propellant a solid or semi-solid composition or gel. In one or more embodiments, the composition or gel is a liquid. In one or more embodiments the propellant is miscible with and dilutes the composition.

Upon packaging of the foamable composition in an aerosol container and adding a propellant, a shakable and homogenous foamable composition is prepared, which upon dispensing forms a breakable foam with good to excellent quality. The resulting foam is pharmaceutically equivalent to the respective gel (prior to adding the propellant), since immediately upon dispensing of the foam the propellant evaporates and the composition upon collapsing is similar or identical to that of the gel. This is an important pragmatic advantage, because many drug development activities, including expensive and lengthy toxicology studies with numerous animals and clinical trials with thousands of patients can be saved by conducting such studies once for either the gel or foam presentation instead of twice (for each presentation).

Application can be, hourly, twelve hourly (e.g., twice daily), daily, alternate-day or intermittent, according to the condition of the patient. For reasons of compliance, less frequent applications, where possible, are preferable, e.g., daily single applications. In certain cases, where prolonged or long term treatment is required, an initial dose is provided followed by a gradual reduction to a lower maintenance dose, which can be increased if further outbreaks occur.

In one or more embodiments, the initial dose of tetracycline is about 18%, or about 17.5%, or about 16.5%, or about 15.5%, or about 14.5%, or about 13.5% or about 12.5%, or about 11.5%, or about 10.5% or about 9.5% or about 8.5% or about 7.5% or about 6.5% or about 5.5% or about 4.5% or about 3.5% or about 2.5% or about 1.5%, or about 17%, or about 16%, or about 15%, or about 14%, or about 13% or about 12%, or about 11%, or about 10% or about 9% or about 8% or about 7% or about 6% or about 5% or about 4% or about 3% or about 2% or about 1% or about 0.75% or about 0.5% or about 0.25% or about 0.2% by weight of the composition. In one or more embodiments, the maintenance dose of tetracycline is about 7.5% or about 6.5% or about 5.5% or about 4.5% or about 3.5% or about 2.5% or about 1.5%, 7% or about 6% or about 5% or about 4% or about 3% or about 2% or about 1% or about 0.5%, or about 1.9%, or about 1.8%, or about 1.7%, or about 1.6%, or about 1.55 or about 1.4% or about 1.3% or about 1.2% or about 1.1%, or about 0.9% or about 0.8%, or about 0.7%, or about 0.6% or about 0.4% or about 0.35 or about 0.25% or about 0.2% or about 0.15% or about 0.1% by weight of the composition.

In one or more embodiments, such a composition is presented as a breakable gel, which breaks down with mild mechanical force.

In one or more embodiments, the hydrophobic composition when packaged in an aerosol container to which is added a liquefied or compressed gas propellant the composition provides upon release from the container a breakable foam of at least good quality that breaks easily upon application of mechanical force.

In one or more embodiments, the composition is a foamable composition that is thermally stable at skin temperature.

In one or more embodiments, when the above composition is filled into an aerosol can or canister and pressurized with a propellant a foamable composition is produced.

In one or more embodiments, the at least one hydrophobic solvent comprises or is selected from the group consisting of a mineral oil, a hydrocarbon oil, an ester oil, an ester of a dicarboxylic acid, a triglyceride oil, an oil of plant origin, an oil from animal origin, an unsaturated or polyunsaturated oil, a diglyceride, a PPG alkyl ether, an essential oil, a silicone oil, liquid paraffin, an isoparaffin, a polyalphaolefin, a polyolefin, polyisobutylene, a synthetic isoalkane, isohexadecane, isododecane, alkyl benzoate, alkyl octanoate, C₁₂-C₁₅ alkyl benzoate, C₁₂-C₁₅ alkyl octanoate, arachidyl behenate, arachidyl propionate, benzyl laurate, benzyl myristate, benzyl palmitate, bis(octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethylhexanoate, diethyleneglycol dioctanoate, diethyleneglycol diisononanoate, diethyleneglycol diisononanoate, diethylhexanoate, diethylhexyl adipate, diethylhexyl malate, diethylhexyl succinate, diisopropyl adipate, diisopropyl dimerate, diisopropyl sebacate, diisosteary dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystarate, ethylhexyl isononanoate, ethylhexyl palmytate, ethylhexyl pelargonate, ethylhexyl stearate, hexadecyl stearate, hexyl laurate, isoamyl laurate, isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isosteary citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isosteary tartarate, isotridecyl isononanoate, isotridecyl isononanoate, lauryl lactate, myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl erucate, oleyl lactate, oleyl oleate, propyl myristate, propylene glycol myristyl ether acetate, propylene glycol dicaprate, propylene glycol dicaprylate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, alexandria laurel tree oil, avocado oil, apricot stone oil, barley oil, borage seed oil, calendula oil, canelle nut tree oil, canola oil, caprylic/capric triglyceride castor oil, coconut oil, corn oil, cotton oil, cottonseed oil, evening primrose oil, flaxseed oil, groundnut oil, hazelnut oil, glycereth triacetate, glycerol triheptanoate, glyceryl trioctanoate, glyceryl triundecanoate, hempseed oil, jojoba oil, lucerne oil, maize germ oil, marrow oil, millet oil, neopentylglycol dicaprylate/dicaprate, olive oil, palm oil, passionflower oil, pentaerythrityl tetrastearate, poppy oil, propylene glycol ricinoleate, rapeseed oil, rye oil, safflower oil, sesame oil, shea butter, soya oil, soybean oil, sweet almond oil, sunflower oil, sysymbrium oil, syzigium aromaticum oil, tea tree oil, walnut oil, wheat germ glycerides, wheat germ oil, PPG-2 butyl ether, PPG-4 butyl ether, PPG-5 butyl ether, PPG-9 butyl ether, PPG-12 butyl ether, PPG-14 butyl ether, PPG-15 butyl ether, PPG-15 stearyl ether, PPG-16 butyl ether, PPG-17 butyl ether, PPG-18 butyl ether, PPG-20 butyl ether, PPG-22 butyl ether, PPG-24 butyl ether, PPG-26 butyl ether, PPG-30 butyl ether, PPG-33 butyl ether, PPG-40 butyl ether, PPG-52 butyl ether, PPG-53 butyl ether, PPG-10 cetyl ether, PPG-28 cetyl ether, PPG-30 cetyl ether, PPG-50 cetyl ether, PPG-30 isocetyl ether, PPG-4 lauryl ether, PPG-7 lauryl ether, PPG-2 methyl ether, PPG-3 methyl ether, PPG-3 myristyl ether, PPG-4 myristyl ether, PPG-10 oleyl ether, PPG-20 oleyl ether, PPG-23 oleyl ether, PPG-30 oleyl ether, PPG-37 oleyl ether, PPG-40 butyl ether, PPG-50 oleyl ether, PPG-11 stearyl ether, herring oil, cod-liver oil, salmon oil, cyclomethicone, a dimethyl polysiloxane, dimethicone, an epoxy-modified silicone oil, a fatty acid-modified silicone oil, a fluoro group-modified silicone oil, a methylphenylpolysiloxane, phenyl trimethicone and a polyether group-modified silicone oil. In some embodiments, the hydrophobic solvent comprises or is selected from the group consisting of soybean oil, a coconut oil, a cyclomethicone, a light mineral oil, and mixtures thereof. In one or more embodiments the solvent is tested individually for compatibility with a tetracycline antibiotic and is only used if it passes a compatibility test as described below in the Methods.

In one or more embodiments, the hydrophobic solvent is at a concentration of about 75% to about 90% by weight. In one or more embodiments, the hydrophobic solvent is at a concentration of at least about 40% by weight, at least about 50% by weight, at least about 60% by weight, at least about 70% by weight, at least about 90% by weight. In some embodiments, the hydrophobic solvent is at a concentration of less than about 90% by weight, less than about 80% by weight, less than about 70% by weight, less than about 60% by weight, less than about 50% by weight.

In one or more embodiments, the viscosity-modifying agent is at a concentration of about 0.1% to about 22%, about 0.4 to about 18%, about 0.5% to 16%, about 0.6% to 14%, about 0.7% to 13%, about 0.8 to about 12%, about 0.9% to about 11%, about 1% to about 10%, about 10% to about 22% by weight. In one or more embodiments, the viscosity-modifying agent is a fatty alcohol having at least 12 carbon atoms in its carbon backbone. In one or more embodiments, the viscosity-modifying agent is a fatty acid having at least 12 carbon atoms in its carbon backbone.

In one or more embodiments, the viscosity-modifying agent is at a concentration of about 9.5% or about 8.5% or about 7.5% or about 6.5% or about 5.5% or about 4.5% or about 3.5% or about 2.5% or about 1.5%, about 7% or about 6% or about 5% or about 4% or about 3% or about 2% or about 1% or about 0.5%, or about 1.9%, or about 1.8%, or about 1.7%, or about 1.6%, or about 1.55 or about 1.4% or about 1.3% or about 1.2% or about 1.1%, or about 0.9% or about 0.8%, or about 0.7%, or about 0.6% or about 0.5% by weight of the composition or less than any of the aforesaid amounts.

In one or more embodiments, the fatty alcohol and/or fatty acid have a melting point of at least about 40° C.

In one or more embodiments, the fatty alcohol comprises or is selected from the group consisting of lauryl alcohol, myristyl alcohol, cetyl alcohol, stearyl alcohol, arachidyl alcohol, behenyl alcohol, tetracosanol, hexacosanol, octacosanol, triacontanol, and tetratriacontanol. In one or more embodiments, the fatty acid comprises or is selected from the group consisting of dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid, and pentatriacontanoic acid.

In one or more embodiments, the carbon chain of the fatty alcohol or the fatty acid is substituted with a hydroxyl group.

In one or more embodiments, the fatty acid is 12-hydroxy stearic acid.

In one or more embodiments, the viscosity-modifying agent is a wax comprising or selected from the group consisting of a plant wax, carnauba wax, candelilla wax, ouricury wax, sugarcane wax, retamo wax, jojoba oil, an animal waxes, beeswax, a petroleum derived wax, a paraffin wax, polyethylene, and derivatives thereof.

In one or more embodiments, the viscosity-modifying agent is a combination comprising (i) at least one fatty alcohol and at least one fatty acid; or (ii) at least one fatty alcohol and at least one wax; or (iii) at least one fatty acid and at least one wax; or (iv) at least one fatty alcohol, at least one fatty acid, and at least one wax.

In one or more embodiments the at least one viscosity-modifying agent comprises or is selected from the group consisting of a fatty alcohol, a fatty acid and a wax, wherein the fatty alcohols and/or fatty acids have at least 12 carbon atoms in their carbon backbone. In certain embodiments the viscosity modifying agent is a combination of a fatty alcohol and a fatty acid and/or a wax.

Preferably, the fatty alcohol and/or fatty acid and/or wax are solid at ambient temperature. In certain embodiments, the fatty alcohol and/or the fatty acid and/or the wax or the mixture of them have a melting point of more than about 40° C.

### Incompatible Excipients and Undesirable Excipients

In certain embodiments, the composition is free of one or more of a petrolatum, surface active agents, protic solvents, certain polar aprotic solvents, isopropyl myristate, polyethylene gelling agents, polyethylene homopolymers, polyethylene copolymers, selenium derivatives and silicone thickening agents; and in certain embodiments, the foamable composition is substantially free of such excipients. In the context herein, the term “substantially-free” relates to a composition that contains a total of less than about 0.4% of a petrolatum, surface active agents, protic solvents, certain polar aprotic solvents, isopropyl myristate, polyethylene gelling agents, polyethylene homopolymers, polyethylene copolymers, selenium derivatives and silicone thickening agents cumulatively. Preferably, the composition comprises less than about 0.2% of two or more or all thereof by weight of petrolatum, surface active agents, protic solvents, certain polar aprotic solvents, isopropyl myristate, polyethylene gelling agents, polyethylene homopolymers, polyethylene copolymers, selenium derivatives and silicone thickening agents cumulatively or, and more preferably less than about 0.1% individually or of two or more or all thereof cumulatively.

### Surface Active Agents

For clarification, in the context herein whilst the term “standard surfactant” or “customary surfactant” refers herein to customary non-ionic, anionic, cationic, zwitterionic, amphoteric and amphiphilic surfactants A fatty alcohol or a fatty acid and certain waxes are not regarded as a standard surfactant. However, in contrast, ethers or esters formed from such fatty alcohols or fatty acids can be regarded as a customary surfactant.

Surfactants of all kinds are undesirable in accordance with the present invention, as (i) they were found to cause degradation of the tetracycline antibiotic; and (ii) they are generally known to possess irritation potential.

Non-limiting examples of classes of non-ionic surfactants that are undesirable according to the present invention include: (i) polyoxyethylene sorbitan esters (polysorbates), such as polysorbate 20, polysorbate 40, polysorbate 60 and polysorbate 80; (ii) sorbitan esters, such as sorbitan monolaurate and sorbitan monooleate; (iii) polyoxyethylene fatty acid esters, such as, PEG-8 stearate, PEG-20 stearate, PEG-40 stearate, PEG-100 stearate, PEG-150 distearate, PEG-8 laurate, PEG-10 laurate, PEG-12 laurate, PEG-20 laurate, PEG-8 oleate, PEG-9 oleate, PEG-10 oleate, PEG-12 oleate, PEG-15 oleate and PEG-20 oleate; (iv) PEG-fatty acid diesters; (v) polyethylene glycol (PEG) ethers of fatty alcohols; (vi) glycerol esters, such as glyceryl monostearate, glyceryl monolaurate, glyceryl monopalmitate and glyceryl monooleate; (vii) PEG-fatty acid mono- and di-ester mixtures; (viii) polyethylene glycol glycerol fatty acid esters; (ix) propylene glycol fatty acid esters; (x) mono- and diglycerides; (xi) sugar esters (mono-, di- and tri-esters of sucrose with fatty acids) and (xii) PEG alkyl phenols.

As mentioned above, in the context of the present invention, while fatty alcohols, fatty acids, and certain waxes are somewhat amphiphilic, these substances are not effective as stand-alone surfactants that can stabilize an emulsion, let alone foamable emulsion compositions, because of their very weak emulsifying capacity and further due to their weak foaming capacity on their own.

They are occasionally used in a supporting role as co-emulsifiers, i.e., in combination with a standard surfactant but are commonly used as thickeners and have successfully been used as foam adjuvants to assist customary surfactants to boost foam quality and stability. For the purposes of forming an emulsion they are usually regarded as an oil and thus have a “required” HLB value for the purpose of determining what standard surfactant might be appropriate to use with the oil phase.

Generally, surfactants are known to possess irritation potential. One way to try and reduce or minimize potential irritation and drying of the skin or mucosa due to surfactants and their repeated use, especially when formulations are to be left on the skin or mucosa rather than being washed off, is to use essentially or primarily nonionic surfactants at significant concentrations although preferably below 5%. The current breakthrough of identifying formulations which produce gels and quality breakable foam yet omitting customary surfactants from a composition may contribute to improved tolerability of such a composition and can be an important advantage. This is especially so when a formulation is to be applied to a very sensitive target site, and particularly so on a repeated basis.

In certain embodiments, the composition is free of customary surfactants, or “surfactant-free” and in certain embodiments the foamable composition is substantially free of customary surfactants, or “substantially surfactant-free”.

In certain embodiments, the composition is free or substantially free of an ionic surfactant. In certain embodiments, the composition is free or substantially free of a zwitterionic surfactant. In certain embodiments, the composition is free or substantially free of a non-ionic surfactant.

### Protic Solvents

Protic solvents, such as short chain alcohols, glycols and glycerin are incompatible with tetracyclines and therefore are undesirable.

### Aprotic Polar Solvents

It was discovered in WO11/039637 that certain polar aprotic solvents are incompatible with tetracycline antibiotics. Thus, aprotic polar solvents, such as dimethyl sulfoxide (DMSO), dimethylformamide (DMF), acetonitrile, acetone, methyl ethyl ketone, 1,4-Dioxane and tetrahydrofuran (THF), N-methylpyrrolidone, pyridine, piperidine, dimethylformanide, N-methyl-2-pyrrolidone and 1-methyl-2-pyrrolidinone) and azone (1-dodecylazacycloheptan-2-one) are undesirable.

### Silicone Thickening Agents

Silicone thickening agents comprise one or more polysiloxane-derived components. Such polysiloxanes are typically cross-linked and they have rubber-like characteristics, which require their solubilization in an oil, usually a silicone oil. An example of such a silicone thickening agent is ST-Elastomer 10 (Dow Corning), which is a mixture of high molecular weight dimethicone crosspolymer (12%), in cyclopentasiloxane (cyclomethicone, silicone solvent). With reference to bioavailability of an active agent in the skin following topical application, it is conceivable that cross co-polymers will create a non-permeable film which should block skin penetration and therefore, it is undesirable. Further, in the context of a breakable foam, cyclomethicone is known as a defoamer and therefore its presence in high concentrations in the breakable hydrophobic composition is undesirable.

In one or more other specific embodiments, the drug carrier is formulated substantially free of elastomers. In one or more other specific embodiments, the drug carrier is formulated essentially free of elastomers. In one or more other specific embodiments, the drug carrier is formulated substantially free of silicones. In one or more other specific embodiments, the drug carrier is formulated essentially free of silicones. In one or more other specific embodiments, the drug carrier is formulated with less than about 30% silicones, or less than about 25% silicones, or less than about 20% silicones, or less than about 15% silicones, or less than about 10% silicones, or less than about 7.5% silicones, or less than about 5% silicones or less than about 2% silicones; or less than about 1% silicones; or less than about 0.5% silicones; or about 1% to about 5% silicones; or about 0.5% to about 3% silicones. In one or more other specific embodiments, the drug carrier does not comprise a silicone other than cyclomethicone. In one or more other embodiments, the drug carrier does not comprise one or more volatile silicones. In other embodiments, volatile silicones are present at about 3% or less.

In one or more embodiments, semi-solid hydrophobic oils are a subsidiary component in the composition, for example being present at less than about 45%, at less than about 40%, at less than about 35%, at less than about 30%, at less than about 25%, less than about 20%, less than about 15%, less than about 10%, or less than about 5% by weight of the composition. In one or more alternative embodiments, semi-solid oils are omitted.

### Polyol

The identification of a “polyol”, as used herein, is an organic substance that contains at least two hydroxy groups in its molecular structure. In one or more embodiments, the polyol is a diol (a compound that contains two hydroxy groups in its molecular structure). Examples of diols include propylene glycol (e.g., 1,2-propylene glycol and 1,3-propylene glycol), butanediol (e.g., 1,2-butanediol, 1,3-butanediol, 2,3-butanediol and 1,4-butanediol), butanediol (e.g., 1,3-butanediol and 1,4-butenediol), butynediol, pentanediol (e.g., pentane-1,2-diol, pentane-1,3-diol, pentane-1,4-diol, pentane-1,5-diol, pentane-2,3-diol and pentane-2,4-diol), hexanediol (e.g., hexane-1,6-diol hexane-2,3-diol and hexane-2,56-diol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiments, the polyol is a triol (a compound that contains three hydroxy groups in its molecular structure), such as glycerin, butane-1,2,3-triol, butane-1,2,4-triol and hexane-1,2,6-triol.

In one or more embodiments, the polyol is a saccharide. Exemplary saccharides include, but are not limited to, monosaccharide, disaccharides, oligosaccharides, and sugar alcohols.

A monosaccharide is a simple sugar that cannot be hydrolyzed to smaller units.

Empirical formula is (CH₂O)ₙ and range in size from trioses (=3) to heptoses (n=7). Exemplary monosaccharide compounds are ribose, glucose, fructose, and galactose.

Disaccharides are made up of two monosaccharides joined together, such as sucrose, maltose, and/or lactose.

In one or more embodiments, the polyol is a sugar alcohol (also known as a polyol, polyhydric alcohol, or polyalcohol) or a hydrogenated form of saccharide, whose carbonyl group (aldehyde or ketone, reducing sugar) has been reduced to a primary or secondary hydroxyl group. They are commonly used for replacing sucrose in foodstuffs, often in combination with high intensity artificial sweeteners to counter the low sweetness. Some exemplary sugar alcohols, which are suitable for use according to the present invention are mannitol, sorbitol, xylitol, maltitol, lactitol. (Maltitol and lactitol are not completely hydrogenated compounds—they are a monosaccharide combined with a polyhydric alcohol.) Mixtures of polyols, including (1) at least one polyol comprises or selected from a diol and a triol; and (2) a saccharide are contemplated within the scope of the present disclosure.

According to some embodiments, the composition is polyol free, i.e. free of polyols.

In other embodiments, the composition is substantially free and comprises less than about 5% final concentration of polyols, preferably less than 2%, more preferably less than 1%; or about 1% to about 5% polyols; or about 0.5% to about 3% polyols. In some embodiments the composition comprises de minimis amounts of polyols. Where a formulation includes insignificant or de minimis amounts of polyols such as less than 0.05% it is considered to be essentially free of them.

In an embodiment, the polyol is linked to a hydrophobic moiety. In the context of the present disclosure, a polyol linked to a hydrophobic moiety is still defined as a “polyol” as long as it still contains two or more free hydroxyl groups.

In an embodiment, the polyol is linked to a hydrophilic moiety. In the context of the present disclosure, a polyol linked to a hydrophilic moiety is still defined “polyol” as long as it still contains two or more free hydroxyl groups.

In one or more embodiments, the hydrophobic composition further contains an anti-infective agent, comprises or selected from the group of an antibiotic agent, an antibacterial agent, an antifungal agent, an agent that controls yeast, an antiviral agent, and an antiparasitic agent. In an embodiment, the anti-infective agent comprises a tricyclic antibiotic. Not only can combining the anti-infective effect of a hydrophobic composition, with an anti-infective agent can result in a synergistic effect and consequently higher success rate of the treatment but the combination with the viscosity modifying agent achieves a formulation in which the active pharmaceutical ingredient is chemically stable and the formulation is physically stable as demonstrated herein in the Examples. Moreover, the use of hydrophobic based water-free formulation can maximize the antimicrobial and antiviral potentials of the formulations. Delivery topically can be improved by using a hydrophobic carrier with a hydrophobic API. Storage in sealed, light and airtight canisters can assist in preserving the formulations.

In one or more embodiments, the hydrophobic composition is substantially free of at least one or more selected from a group consisting of surface active agents, protic solvents, polar aprotic solvents, and silicone thickening agents.

In one or more embodiments, the hydrophobic composition is substantially free of at least one or more selected from a group consisting of surface active agents, polymeric gelling agents, polyols, short chain alcohols, and silicone thickening agents.

In one or more embodiments, the hydrophobic composition contains less than about 0.4% by weight of the composition; or less than about 0.2% by weight of the composition; or less than about 0.1% by weight of the composition of one of or a combination of two, three or all of surface active agents, protic solvents, polar aprotic solvents, and silicone thickening agents.

### The Composition Essential Ingredients

In certain embodiments, a hydrophobic solvent can be useful. For example, some essential oils can kill microorganisms or may prevent of conditions that involve microbial infection. Additionally, hydrophobic solvents can useful for the treatment of conditions which involve damaged skin, such as psoriasis or atopic dermatitis. The combination of a hydrophobic solvent and a fatty alcohol or fatty acid may be of possible help in conditions characterized, for example, by infection and/or inflammation.

Fatty alcohols can also be of possible help. Long chain saturated and mono unsaturated fatty alcohols, e.g., stearyl alcohol, erucyl alcohol, arachidyl alcohol and behenyl alcohol (docosanol) have been reported to possess antiviral, antiinfective, antiproliferative and anti-inflammatory properties (see, U.S. Pat. No. 4,874,794). Longer chain fatty alcohols, e.g., tetracosanol, hexacosanol, heptacosanol, octacosanol, triacontanol, etc., are also known for their metabolism modifying properties, and tissue energizing properties.

In one or more embodiments, the active agent may be a placebo or a cosmetic agent. The foamable composition is suitable for use in the manufacture of a medicament including a placebo or active agent.

### Combination of Active Agents

Several disorders involve a combination of more than one etiological factor; and therefore, the use of more than one active agent is advantageous. For example, psoriasis involves excessive cell proliferation and inadequate cell differentiation as well as inflammation. Atopic dermatitis involves keratinocyte growth abnormality, skin dryness and inflammation. Bacterial, fungal and viral infections involve pathogen colonization at the affected site and inflammation. Hence, in many cases, the inclusion of a combination of active agents in the pharmaceutical composition can be desirable. Thus, in one or more embodiments, the composition includes at least two active agents, in a therapeutically effective concentration.

In one or more embodiments, a combination of any two or more of an antibacterial, an antiinflamitory, an antifungal, and an antivirial agent is contemplated.

In one or more embodiments, a tetracycline antibiotic is the sole active ingredient present in the composition. In one or more embodiments, a minocycline is the sole active ingredient present in the composition. In one or more embodiments, a doxycycline is the sole active ingredient present in the composition. In one or more embodiments minocycline and doxycycline are used in combination.

In one or more embodiments, a combination of any two or more of a minocycline, retinoids, and benzoyl peroxide is contemplated

In one or more embodiments, a combination of any two or more of a tetracyline, retinoids, and benzoyl peroxide is contemplated.

In one or more embodiments, a combination of any two or more of benzoyl peroxide, antibiotics, retinoids, antiseborrheic medications, anti-androgen medications, hormonal treatments, salicylic acid, alpha hydroxy acid, azelaic acid, nicotinamide, and a keratolytic agent is contemplated.

In one or more embodiments, the tetracycline antibiotic comprises or is selected from the group consisting of tetracycline, oxytetracycline, demeclocycline, doxycycline, lymecycline, meclocycline, methacycline, minocycline, rolitetracycline, chlorotetracycline, and tigecycline.

In one or more embodiments, the tetracycline antibiotic is hydrophobic.

In one or more embodiments, the Log of the distribution constant of the tetracycline antibiotic at pH 7.0 (buffer/chloroform) is equal to or less than about 0.2.

In one or more embodiments, the tetracycline antibiotic is present in a free base form, a hydrate form, a salt form, a chelate complex form or a coordination complex form.

In one or more embodiments, the tetracycline antibiotic does not comprise a hydroxy group at carbons 5, 6, and 7.

In one or more embodiments, the tetracycline antibiotic comprises or is selected from the group consisting of minocycline and doxycycline. In some embodiments, the tetracycline antibiotic is minocycline. In some embodiments, the concentration of minocycline is in a range between about 0.1% to about 10% by weight (e.g., about 0.1% to about 8% by weight, about 0.1% to about 5% by weight, about 0.1% to about 3% by weight, about 0.1% to about 2% by weight, about 0.10% to about 10% by weight, about 0.10% to about 0.75% by weight, about 0.1% to about 0.5% by weight, about 0.1% to about 0.25% by weight, about 0.25% to about 10% by weight, about 0.5% to about 10% by weight, about 1% to about 10% by weight, about 2% to about 10% by weight, about 4% to about 10% by weight, about 6% to about 10% by weight, about 7% to about 10% by weight, about 8% to about 10% by weight, about 0.5% to about 2.0% by weight, about 0.75% to about 1.5% by weight, about 1% to about 3% by weight, about 1% to about 4% by weight, and about 2% to about 6% by weight). In some embodiments, the concentration of minocycline is at least about 0.05% by weight, is at least about 0.1% by weight, at least about 0.5% by weight, at least about 1% by weight, at least about 2% by weight, at least about 4% by weight, at least about 6% by weight, at least about 8% by weight or at least about 10% by weight.

The topical compositions of the present invention avoid, reduce, minimize or or do not cause adverse effects, which are attributed to oral tetracycline antibiotics. Photosensitivity, for example, is a known side effect of oral minocycline. It is manifested as an exaggerated sunburn reaction on areas of the body exposed to direct sunlight or ultraviolet light, resulting in muddy brown skin discoloration. Use of oral minocycline over an extended period of time can also lead to skin pigmentation e.g. manifested as blue-gray skin and blue-gray staining in areas of scaring and inflammation associated with acne. Tooth staining potential of oral minocycline in adult populations has also been acknowledged in recent literature. In contrast, no tooth staining was reported during the period of topical application of T % or 4% minocycline foam or on follow-up. So in one or more embodiments topical minocycline avoids tooth staining.

Surprisingly, it has been previously demonstrated by Applicants in U.S. Ser. No. 13/499,475 minimal to no skin pigmentation following rubbing of 4% minocycline foam onto the skin was noticed, when observed after about 30 seconds. It has now been surprisingly further discovered that no photosensitivity or skin discoloration was noticed following application of 1% or 4% minocycline foam onto the skin once daily for 12 weeks. Similarly, drug related pigmentation was not observed.

This further corroborates and strengthens previous findings that the composition of the present invention had protective properties in the case of UVB-induced sun damage or any other condition associated with sunlight or other light (e.g., laser) exposure. Applicants formulations and methods of treatment may therefore be able to reduce skin photodamage and photoaging, and more generally to reduce oxidative stress and inflammation in skin pathologies which are known to be accompanied by apoptotic cell death.

In one or more embodiments the method is useful for treating a disorder, including administering topically to a surface having the disorder a hydrophobic composition as described above, wherein:
(a) the at least one hydrophobic solvent comprises or is selected from a group consisting of a soybean oil, a coconut oil, a cyclomethicone, a light mineral oil, and mixtures thereof;
(b) the at least one viscosity modifying agent comprises or is selected from a group consisting of a fatty acid, a fatty alcohol, a wax, a hydrogenated castor oil, and mixtures thereof; and
(c) the tetracycline antibiotic is minocycline, or a salt thereof, such as minocycline HCl.

In one or more embodiments the disorder is acne or acne related or has acne like symptoms.

In one or more embodiments, the composition further comprises fumed or modified silica (SiO₂) such as Aerosil R972.

In one or more embodiments, the minocycline is micronized.

In one or more embodiments, the composition is a foamable composition, and further comprises a propellant. Any compatible propellant may be used. In one or more embodiments, the propellant is a gas at room temperature under normal pressure and which may be liquefied at increased pressure at room temperature. Examples of propellants include, without limitation, hydrocarbon propellants such as butane, propane, isobutane, dimethyl ether, fluorocarbons such as 1,1,1,2 tetrafluorethane (Dymel 134), and 1,1,1,2,3,3,3 heptafluoropropane (Dymel 227), and mixtures thereof. In one or more embodiments, a hydrocarbon mixture AP-70 (a mixture of about 30% w/w butane, 20% w/w isobutane and 50% w/w propane) is used.

In one or more embodiments of the invention, there is disclosed a method for treating a disorder of the pilosebaceous unit, including acne, including administering topically to a surface having the disorder a hydrophobic composition substantially free of surfactants, and/or substantially free of surfactants and polymeric agents as described above, wherein
(a) the at least one hydrophobic solvent comprises or is selected from a group consisting of a soybean oil, a coconut oil, a cyclomethicone, a light mineral oil, and mixtures thereof;
(b) the fatty alcohol comprises or is selected from a group consisting of cetostearyl alcohol, myristyl alcohol, stearyl alcohol, behenyl alcohol, and mixtures thereof;
   the fatty acid comprises or is selected from the group consisting of stearic acid, beeswax, a hydrogenated castor oil, and mixtures thereof:
   the wax comprises or is selected from the group consisting of bees wax, a hydrogenated castor oil, and mixtures thereof; and
   the tetracycline antibiotic is selected from a group consisting of a minocycline and a doxycycline.

In one or more embodiments the above hydrophobic composition is used to treat one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections.

In one or more embodiments, the tetracycline antibiotic is minocycline HCl.

In one or more embodiments, the tetracycline antibiotic is doxycycline hyclate.

Also provided herein is a method for treating human skin diseases or disorders especially for the treatment of acne, rosacea and/or superficial infections, including skin infections, such as impetigo, including administering topically to a surface having the disease or disorder a hydrophobic composition containing:
(a) a mixture of soybean oil in an amount of about 50 weight percent, coconut oil in an amount of about 24 weight percent, cyclomethicone in an amount of about 5 weight percent, and light mineral oil in an amount of about 4 weight percent;
(b) a mixture of about 3.5 weight percent cetostearyl alcohol, about 2.5 weight percent myristyl alcohol, about 1.5 weight percent stearyl alcohol, about 1 weight percent behenyl alcohol, about 3 weight percent stearic acid, about 2 weight percent beeswax, and about 2 weight percent hydrogenated castor oil;
(c) fumed (modified) silica in an amount of about 0.25 weight percent; and
(d) minocycline HCl in an amount of about 1.0 weight percent.

In one or more embodiments of the invention is disclosed a method for treating human skin disorders or diseases. In one or more embodiments a method of treating one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and superficial infections, including skin infections, including administering topically to a surface having the disorder a hydrophobic composition substantially free of surfactants, and/or substantially free of surfactants and polymeric agents as described above, containing:
(a) a mixture of soybean oil in an amount of about 50 weight percent, coconut oil in an amount of about 23.6 weight percent, cyclomethicone in an amount of about 5 weight percent, and light mineral oil in an amount of about 1 weight percent;
(b) a mixture of about 3.5 weight percent cetostearyl alcohol, about 2.5 weight percent myristyl alcohol, about 1.5 weight percent stearyl alcohol, about 1 weight percent behenyl alcohol, about 3 weight percent stearic acid, about 2 weight percent beeswax, and about 2 weight percent hydrogenated castor oil;
(c) modified (fumed) silica (Aerosil R 972) in an amount of about 0.25 weight percent; and
(d) minocycline HCl (micronized) in an amount of about 4.44% weight percent.

In one or more embodiments, any composition of the present invention can also contain a fragrance. In one or more embodiments, the fragrance is at a concentration of about 0.1% by weight to about 1% by weight.

In one or more embodiments, the composition comprises about 48% w/w to about 51% w/w of soybean oil. In one or more embodiments, the composition comprises about 23% w/w to about 24% w/w of coconut oil. In one or more embodiments, the composition comprises about 4% w/w to about 6% w/w of cyclomethicone. In one or more embodiments, the composition comprises about 1% w/w to about 5% w/w of light mineral oil.

In one or more embodiments, the composition comprises about 3% w/w to about 4% w/w of cetostearyl alcohol. In one or more embodiments, the composition comprises about 2% w/w to about 4% w/w of stearic acid. In one or more embodiments, the composition comprises about 2% w/w to about 3% w/w of myristyl alcohol. In one or more embodiments, the composition comprises about 1% w/w to about 2% w/w of stearyl alcohol. In one or more embodiments, the composition comprises about 0.5% w/w to about 1.5% w/w of behenyl alcohol. In one or more embodiments, the composition comprises about 1% w/w to about 3% w/w of hydrogenated castor oil. In one or more embodiments, the composition comprises about 1% w/w to about 3% w/w of beeswax.

In one or more embodiments, the composition comprises about 0.1% w/w to about 0.3% w/w of fumed (modified) silica. In one or more embodiments, the composition comprises about 1% w/w to about 4% w/w of minocycline hydrochloride or a doxcycline or a tetracycline antibiotic. In one or more embodiments, the composition comprises about 3% w/w to about 15% w/w of propellant based on the weight of the total composition.

In one or more embodiments, there is provided a method for treating acne vulgaris, including administering topically to a surface having acne vulgaris a composition which is highly effective against bacteria. In one or more embodiments the tetracycline antibiotic is effective against some multi-drug resistant strains (e.g., antibiotic-resistant P. acnes).

In one or more embodiments, there is provided a method for treating acne vulgaris, including administering topically to a surface having acne vulgaris a composition which is highly effective against antibiotic-resistant P. acnes bacteria. In some embodiments, the composition comprises a tetracycline antibiotic and a vehicle. In some embodiments, the vehicle is also effective in treating skin with acne.

In one or more embodiments, there is provided a method for treating acne vulgaris, including administering topically, once a day, to a surface having acne vulgaris a composition comprising a tetracycline antibiotic.

In one or more embodiments, there is provided a method for treating acne vulgaris, including administering topically, twice a day, to a surface having acne vulgaris a composition comprising a tetracycline antibiotic.

In one or more embodiments, there is provided a method for treating acne vulgaris, including administering topically, alternate-day or intermittently, to a surface having acne vulgaris a composition comprising a tetracycline antibiotic.

In one or more embodiments, there is provided a method for treating acne vulgaris, including administering topically, gradual reduction to a lower maintenance dose, which can be increased if further outbreaks occur, to a surface having acne vulgaris a composition comprising a tetracycline antibiotic. In one or more embodiments, a maintenance dose can be applied topically, daily, alternate daily, twice weekly or weekly for a month, two months, quarterly, six months or indefinitely. A maintenance dose can include about 0.9%, or about 0.8%, or about 0.7%, or about 0.6%, or about 0.5%, or about 0.4%, or about 0.3%, or about 0.2%, or about 0.1%, or about 0.09%, or about 0.08%, or about 0.07%, or about 0.06%, or about 0.05% by weight of a tetracycline antibiotic. In one or more embodiments, the maintenance dose may be commenced after four weeks of treatment, or after five weeks of treatment, or after six weeks of treatment, or after seven weeks of treatment, or after eight weeks of treatment, or after nine weeks of treatment, or after ten weeks of treatment, or after eleven weeks of treatment, or after twelve weeks of treatment, or after thirteen weeks of treatment, or after fourteen weeks of treatment, or after fifteen weeks of treatment, or after sixteen weeks of treatment.

In one or more embodiments, there is provided a method for treating acne vulgaris, including administering topically, once daily for at least six weeks, to a surface having acne vulgaris a composition comprising a tetracycline antibiotic.

In one or more embodiments, there is provided a method for treating acne vulgaris, including administering topically, once daily upto six weeks, to a surface having acne vulgaris a composition comprising a tetracycline antibiotic.

In one or more embodiments, there is provided a method for treating acne vulgaris, including administering topically, once daily for twelve weeks or less than twelve weeks, to a surface having acne vulgaris a composition comprising a tetracycline antibiotic.

In one or more embodiments, there is provided a method for treating acne vulgaris, including administering topically, once daily for six weeks or less than six weeks, to a surface having acne vulgaris a composition comprising a tetracycline antibiotic.

In one or more embodiments, there is provided a method for treating acne vulgaris, including administering topically, once daily for three weeks or less than three weeks, to a surface having acne vulgaris a composition comprising a tetracycline antibiotic.

In one or more embodiments, there is provided a method for treating a condition involving inflammation of the skin or mucosa (the disorder), including administering topically, once daily for six weeks or less than six weeks, to a surface having the disorder a gel of foam composition comprising a tetracycline antibiotic.

The compositions provided herein are manufactured according to the methods described in the art and as described in Example 1. Gels are usually packaged in a tube but can also be packaged in any other convenient delivery form including for example, bottles with a pump mechanism or canisters such as bag in can devices where propellant is separate from the gel. Foam formulations are usually packed in a container with an outlet valve e.g. aerosol canister. Possible containers and valves are likewise described in the literature as known by those skilled in the art.

In one or more embodiments, the composition is substantially alcohol-free, i.e., free of short chain alcohols having up to 5 carbon atoms in their carbon chain skeleton. In other embodiments, the composition comprises less than about 5% by weight final concentration of short chain alcohols, for example, less than 2% by weight, or less than 1% by weight. In certain embodiments, the composition is free or substantially free of ethanol, propanol, butanol and pentanol.

One known disadvantage of state of the art compositions (for example Retin A) is that they must be administered for at least seven weeks before consistent beneficial effects are observed, thus making it burdensome for use. It is therefore an advantage of the compositions provided herein is that they can be effective when administered once daily for only six weeks or less. In certain embodiments, the composition may further be effective even if administered alternate-day according to the condition of the patient. In other embodiments, the composition may be used even if administered more than once a day and/or for more than twelve weeks according to the condition of the patient and the concentration of the minocycline.

Another disadvantage of state of the art compositions (such as Akne Mycine) is that they have an ointment base, comprising petrolatum which is greasy and generally considered less usable in the case of facial treatment of acne. Another disadvantage of state of the art compositions is that they contain surfactants, which can be irritants. In some cases, irritation at the application site has been reported with the use of such compositions.

It is therefore an advantage of the compositions provided herein that they are breakable gels or foams; and therefore are easy to apply to the skin and also avoid skin irritation that has been associated with compositions containing surfactants.

Therapeutic topical compositions must stay on the skin for a sufficient period of time to allow the active agent to be absorbed onto the skin, to perform its activity and to further exert a preventative effect. They should preferably not irritate the skin; and they should be perceived by the patient as pharmaceutically convenient in order to achieve sufficient patient compliance. By “pharmaceutically convenient”, it is meant that the skin look and feel to the patient is good, i.e., it must not be too watery or too greasy and it must easily be applied.

Foam is advantageous in the topical treatment of skin diseases, especially in teenagers with skin afflicted with acne, since it is light and easy to apply and collapses and spreads with a minor mechanical force like a simple rub. When dispensed, even in small quantities, drug delivery in the form of foam can also cover a larger surface area of application while also facilitating better product application in areas where conventional topical products cannot be as effective. Foam absorbs rapidly—without the need of repeated rubbing—which is helpful and important for treatment of damaged or irritated skin, sores, and lesions. As the composition is absorbed quickly this may contribute to a positive treatment effect by the vehicle alone, or when in combination with the active a higher percentage effect by the active may be observed.

Thermally stable foam which breaks upon application of mild shear force is extremely advantageous in the topical treatment of skin diseases. It can be applied directly onto skin or hands of the patient without collapsing. So hydrophobic compositions according to the description provided herein, facilitate easy application and even distribution of the active agent, thereby improving treatment convenience. In contrast, Evoclin foam is a temperature sensitive foam that collapses immediately on the skin so it must first be applied onto a cool surface and then quickly applied using fingertips onto the surface which impedes patient compliance.

The formulation packaged into an aerosol container is devoid of any contact with air, light, or any other form of contamination as it is a completely sealed system throughout the life of the product. Thus, light and oxidation sensitive actives can be stabilized effectively in the aerosol system.

In one or more embodiments there is provided a method of administering a tetracycline foam composition to a target area such as skin of a patient comprising releasing foam, applying it to the area, and collapsing the foam. In one or embodiments, the foam is applied by spreading. In the course of spreading mechanical shear can cause the foam to collapse. In one or more embodiments, the collapsed foam is not washed off. In one or more embodiments it is absorbed onto the area of skin. In one or more embodiments it avoids skin irritation or an ointment sensation.

Breakable gels, which comprise liquid oils and a thickening agent, are also very convenient for use, as they liquefy on application of mild shear force such as gentle rubbing, and in turn, they readily absorb onto the skin.

In one or more embodiments, there is provided a method of applying a tetracycline gel composition to an area of skin of a patient comprising releasing a gel, applying it to the area, and collapsing or liquefying the gel. In one or more embodiments, the collapsed or liquefied gel is not washed off. In one or more embodiments, the collapsed or liquefied gel is readily absorbed and does not leave an ointment sensation.

In one or more embodiments, a gel or a liquid gel or a collapsed foam is absorbed within 240 seconds, or within 200 seconds, or within 180 seconds, or within 150 seconds, within 120 seconds, or within 100 seconds, or within 80 seconds, or within 60 seconds, or within 50 seconds, or within 40 seconds, or within 30 seconds, or within 20 seconds, or within 10 seconds, or within 5 seconds, or within 2 seconds or less. By absorbed is meant that the composition enters onto and into an area of skin, mucosa or eye, often forming a thin coating on the surface.

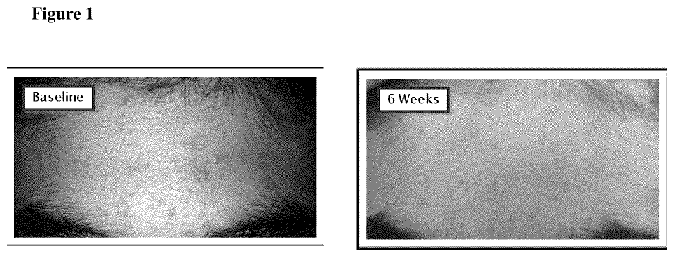

In a preliminary study it has surprisingly been shown that hydrophobic compositions according to the description provided herein have beneficial properties in the treatment of acne vulgaris (see Example 2). According to this preliminary study, following six weeks of treatment, in which the foamable composition containing 1% minocycline was applied once a day on the forehead of a fourteen year old male afflicted with acne vulgaris, an unexpected decrease in the number of both inflammatory and non-inflammatory lesions as well as a significant improvement in the skin condition was observed (See FIG. 1). Surprisingly, no serious adverse events or skin irritation events such as pigmentation, erythema, dryness, itching or peeling were observed. These preliminary results indicate that these compositions might be as effective as, or more effective than the alternatives without untoward reactions.

It was surprisingly shown that therapeutic effects were achieved with low concentrations minocycline such as 1%. Thus, it is possible to use lower concentrations of minocycline thereby reducing toxicity and increasing safety. A number of other skin disorders and diseases can be treated with the composition according to the present invention such as rosacea, wounds, burns, inflammatory skin dermatoses superficial infections, including skin infections, such as impetigo, antibiotic responsive dermatoses and sebaceous gland disorders. The improvement was apparent as was also the restoration of visible, normal cutaneous topographic features, indicating the return of skin integrity as shown in FIG. 1.

Minocycline acts to eradicate P. acnes and reduce the inflammation, thereby reducing the number of inflammatory acne lesions. Minocycline may also have skin regenerating and healing properties responsible for restoration of skin integrity. The combination of minocycline together with a hydrophobic solvent and a fatty alcohol or fatty acid may afford a beneficial effect in conditions characterized, for example, by infection and/or inflammation.

In one or more embodiments, there is provided a method for treating acne (the disorder), including administering topically, to a surface having the disorder, a composition comprising a tetracycline antibiotic, wherein a reduction in the number of lesions is observed after six weeks or less than six weeks of treatment compared to baseline. In one or more embodiments, there is provided a method for treating acne, including administering topically, to a surface having the disorder, a composition comprising a tetracycline antibiotic, wherein an improvement in the skin condition is observed after six weeks or less than six weeks of treatment and wherein an improvement is considered as restoration of visible, normal cutaneous topographic features, indicating the return of skin integrity. In an embodiment the improvement is after three weeks, or after four weeks, or after five weeks, or after six weeks or after seven weeks, or after eight weeks, or after nine weeks, or after ten weeks, or after eleven weeks, or after twelve weeks.

In one or more embodiments, there is provided a method for eradicating P. acnes and reducing inflammation, thereby reducing the number of inflammatory acne lesions. In one or more embodiments, there is provided a method for reducing the number of non-inflammatory acne lesions, by applying topically an effective amount of a tetracycline gel, liquid gel or foam to an afflicted area of a patient in need. In one or more embodiments, the method involves applying a gel, liquid, gel or foam formulation topically to a target surface in need of treatment and breaking the gel or foam over the target site. In one or more embodiments the gel or foam is collapsed and spread by application of a mechanical force, which can be mild or slight such as a simple rub and the active agent is then absorbed. In one or more embodiments the foam or gel is absorbed. In one or more embodiments, the method uses a once daily dosage regime for twelve weeks or less than twelve weeks. In one or more embodiments the twelve week dosage regime is followed by a once daily maintenance dose for one, two, three, four or more weeks according to the condition and response of the patient. In one or more embodiments, the method uses a once daily dosage regime for six weeks or less than six weeks. In one or more embodiments the six week dosage regime is followed by a once daily maintenance dose for one, two, three, four or more weeks according to the condition and response of the patient. In one or more embodiments, the method uses a once daily dosage regime of for six weeks or less than six weeks followed by a once weekly maintenance dose for one, two, three, four, five, six, seven, eight, nine, ten, eleven or more weeks according to the condition and response of the patient. In one or more embodiments, the method uses a once daily dosage regime of for three weeks or less than three weeks followed by a once weekly maintenance dose for one, two, three, four, five, six, seven, eight, nine, ten, eleven or more weeks according to the condition and response of the patient. In one or more embodiments, the method uses a once daily dosage regime of for two weeks followed by a daily maintenance dose for one, two, three or more weeks according to the condition and response of the patient. In one or more embodiments the method uses a once daily dosage regime of for twelve weeks wherein the treatment is every alternate week.

In one or more embodiments, the method uses an additional step of pre cleaning and drying the lesions and surrounding area before applying the gel, liquid gel or foam.

In one or more embodiments, the method uses a sterile applicator or prior to the steps of administering and/or collapsing and/or spreading, the hands of the person spreading are sterilized in order to avoid cross contamination.

In one or more other embodiments, the method uses an additional step of applying an active agent selected from a group consisting of a hyaluronic acid or a retinoid or BPO or salicylic acid, or an alpha hydroxy acid, or azelaic acid, or nicotinamide, or a keratolytic agent to the lesions and surrounding area after the gel, liquid gel or foam has been absorbed. In certain embodiments the active agent selected from a group consisting of a hyaluronic acid, a retinoid, BPO, salicylic acid, an alpha hydroxy acid, azelaic acid, a nicotinamide, a keratolytic agent and mixtures of two or more thereof is applied once daily at least 1, or, 2 or 3 or 4 or 5 or 6 or 7 or 8 or 9 or 10 or 11 or 12 hours after the tetracycline antibiotic formulation has been absorbed. In one or more embodiments, the active agent selected from a group consisting of a hyaluronic acid or a retinoid or BPO or salicylic acid, or an alpha hydroxy acid, or azelaic acid, or nicotinamide, or a keratolytic agent is applied after the third day. In one or more embodiments, the active agent selected from a group consisting of a hyaluronic acid or a retinoid or BPO or salicylic acid, or an alpha hydroxy acid, or azelaic acid, or nicotinamide, or a keratolytic agent is applied during the maintenance stage. In an alternative embodiment the active agent selected from a group consisting of a hyaluronic acid or a retinoid or BPO or salicylic acid, or an alpha hydroxy acid, or azelaic acid, or nicotinamide, or a keratolytic agent is replaced with or supplemented by a steroid.

In an alternative embodiment the active agent selected from a group consisting of a hyaluronic acid or a retinoid or BPO or salicylic acid, or an alpha hydroxy acid, or azelaic acid, or nicotinamide, or a keratolytic agent or steroid is replaced with or supplemented by an antibiotic. In an embodiment the antibiotic, which is in addition to one or more tetracycline antibiotics, is selected from the group consisting of mupirocin, fusidic acid, a penicillin or penicillin derivative, augmentin, an antistaphylococcal penicillin, amoxicillin/clavulanate, a cephalosporin, cephalexin, a macrolide, erythromycin, clindamycin, trimethoprim-sulfamethoxazole penicillin, retapamulin, and mixtures of any two or more thereof. In an embodiment the antibiotic is applied topically. In another embodiment it is applied orally or by injection or by infusion. In another embodiment more than one antibiotic is applied. For example one is applied topically and another is given orally. The latter may be appropriate for example where there is a systemic as well as a topical bacterial infection.

Both the minocycline and the foamable compositions containing minocycline were manufactured under current Good Manufacturing Principles (cGMP) conditions. The foamable composition was provided in aluminum aerosol canisters mounted with valve and actuator. Each canister was filled with 25 g of product and 3 g of propellant. Upon actuation of the canister an aliquot of quality foam was released.

The stability of foamable composition containing minocycline was monitored at 5° C. and 25° C. during and after the clinical trials and satisfactory stability results were obtained (se e.g. Example 3). A multi-center, randomized, double blind, placebo controlled, parallel group, dose finding Phase II clinical study conducted in patients afflicted with acne is reported in Example 3 below. The study was designed to assess the efficacy, safety and tolerability of foamable composition comprising minocycline at one of two different concentrations (strengths): a lower concentration of minocycline of 1% by weight of the formulation and higher concentration of minocycline 4% by weight of the formulation, in comparison with a placebo. The placebo-control design eligibility criteria and endpoints was based on the FDA guidance designated “Guidance for Industry—Acne Vulgaris: Developing Drugs for Treatment (2005).”. The concentrations of minocycline in the composition were selected according to formulation integrity and stability considerations.

The study included six scheduled study visits in which the patients were evaluated: Day 1 (Visit 1) Baseline which included, screening and treatment initiation, Week 3 (Visit 2), Week 6 (Visit 3), Week 9 (Visit 4), Week 12 (Visit 5) End of Treatment (EOT) and Week 16 (Visit 6) “follow up” (F/U). Safety, tolerability and efficacy evaluations were done at baseline, interim visits (2 to 4) and EOT.

At the end of the study no serious adverse events were recorded in the one hundred and thirty nine patients who completed twelve weeks of treatment, which indicates an enhanced safety of topical foamable minocycline compositions. Similarly, no drug related adverse events were noted throughout the study.

Overall, no statistically significant differences were demonstrated between treatment groups in any of the selected skin tolerance parameters during the course of the present study. A few cases of erythema, dryness, peeling, were reported during the 12 weeks of treatment but they were mostly mild and transient and all the patients completed the study. No incidents of drug related pigmentation or itching were reported. These results indicate a satisfactory and enhanced tolerability and safety profile for topical foamable minocycline compositions.

Most of the patients demonstrated a substantial decrease in the number of inflammatory and non-inflammatory lesions, with a percentage decrease higher than that disclosed for oral minocycline. Thus, the results indicate that minocycline foam is at least as effective if not more effective than oral minocycline or other available topical formulations for acne.

Daily application of topical minocycline foam (4% and 1%) on facial skin with moderate to severe acne resulted in a a clinically and statistically significant reduction in the number of inflammatory, non-inflammatory and sum (total) acne lesions; and clinically and statistically significant improvement in the investigator global assessment of acne severity after 12 treatment weeks in the subjects receiving minocycline foam compared to Placebo. The effect of the drug was dose dependent, the effect of 4% minocycline foam was generally greater than 1% minocycline foam and also better than the placebo. It follows that increasing dose between 1 to 4%, for Example, about 1.5%, about 2%, about 2.5%, about 3%, about 3.5%, would respectfully be expected to have a generally increased effect and vice versa. Similarly, increasing the dose above 4% may have an increased effect and reducing it below 1% a decreased effect.

The percentage of decrease from baseline in the mean number of inflammatory lesions in subjects treated with 4% minocycline was statistically significant compared to placebo already after three weeks of treatment and onwards.

The percentage of decrease from baseline in the mean number of non-inflammatory lesions in subjects treated with 4% was statistically significant compared to placebo at twelve weeks.

The percentage of decrease from baseline in the mean number of total lesions in subjects treated with 4% was statistically significant compared to placebo already after six weeks of treatment and onwards.

The percentage of patients having an “almost clear” or “clear” IGA (less than IGA=2) in subjects treated with 4% was statistically significant compared to placebo already after nine weeks of treatment and onwards. Improvement of at least 2 grades in the investigator's global assessment score was also statistically significant more frequent in subjects receiving the 4% minocycline foam compare to subjects in the Placebo group.

After twelve weeks of treatment, approximately two thirds of subjects who received 4% minocycline foam, half of the subjects who received the 1% minocycline foam had an ‘excellent’ improvement, compared to third of subjects in the Placebo group. More than half of subjects who received the 4% minocycline foam evaluated their acne as “much better than prior to study”, compared to 27.6% who received the Placebo. The evaluations were statistically significant.

Notably, more than a 70% reduction in inflammatory lesion counts was reached following 6 weeks of treatment in subjects receiving the 4% minocycline foam.

These results when taken together with the Preliminary Clinical study surprisingly indicate the potential for successful treatment of skin infections such as acne vulgaris with Applicants' topical foamable compositions containing minocycline as an alternative to oral minocycline compositions or other available topical formulations. Thus, Applicants' treatment methods with minocycline in a topical formulation can avoid or minimize unwanted adverse effects seen when given orally or in other topical formulations or methods that result in systemic delivery rather than targeted delivery to the skin in need of treatment.

Topical delivery also means that lower doses can be used again contributing to the elimination or reduction of unwanted side effects. Accordingly, these foamable compositions are expected to be beneficial for the treatment of a range of skin conditions, including rosacea, wound, burn, inflammation, superficial infections, antibiotic responsive diseases or dermatoses, a skin disease caused by bacteria and other skin infections, such as impetigo. Likewise, these foamable compositions are expected to be beneficial in mucosal infections and in eye infections and inflammatory conditions.

In one or more embodiments there is provided a method for treating acne (the disorder), including administering topically, to a surface having the disorder, a composition comprising a tetracycline antibiotic, wherein an enhanced safety and good tolerability of the topical foamable minocycline compositions is demonstrated.

In certain embodiments the treatment is applied on average once daily for two weeks, or for three weeks or for four weeks or for five weeks or for six weeks or for seven weeks or for eight weeks or for nine weeks or for ten weeks or for eleven weeks of for some other period of less than twelve weeks.

In one or more embodiments there is provided a method for treating acne (the disorder), including administering topically, to a surface having the disorder, a composition comprising a tetracycline antibiotic, wherein essentially no skin irritation or essentially no adverse events or no serious adverse events are observed. In one or more embodiments good tolerability was demonstrated with relatively few reports of skin irritation including, erythema, dryness or peeling. In one or more embodiments good tolerability was demonstrated with relatively few or no reports of pigmentation or itching.

In one or more embodiments there is provided a method for treating acne, including administering topically, to a surface having acne, a composition comprising a tetracycline antibiotic, wherein an enhanced efficacy of the topical foamable minocycline compositions is demonstrated.

In one or more embodiments, there is provided a method for treating acne vulgaris, including administering topically to a surface having acne vulgaris a composition comprising a tetracycline antibiotic, wherein after twelve weeks of treatment, at least about 40% of the treated acne vulgaris lesions disappear (in other words, a 40% decrease in the number of lesions) so that no further antimicrobial therapy is necessary. In some embodiments, at least about 50%, at least about 60%, at least about 70% or at least about 80% of the treated acne vulgaris lesions disappear. In one or more embodiments, at least about 90% of the treated acne vulgaris lesions disappear.

In other embodiments, a decrease of at least about 60%, in the number of inflammatory acne vulgaris lesions is observed in at least 65% of the patients after twelve weeks or less than twelve weeks of treatment. In other embodiments, a decrease of at least about 60%, in the number of non-inflammatory acne vulgaris lesions is observed in at least 65% of the patients after twelve weeks or less than twelve weeks of treatment. In one or more other embodiments, a decrease of at least about 60%, in the number of inflammatory and non-inflammatory acne vulgaris lesions is observed in at least 65% of the patients after twelve weeks or less than twelve weeks of treatment. In one or more other embodiments, a decrease of at least about 80%, in the number of inflammatory acne vulgaris lesions is observed in at least 33% of the patients after twelve weeks or less than twelve weeks of treatment. In one or more other embodiments, a decrease of at least about 80%, in the number of non-inflammatory acne vulgaris lesions is observed in at least 25% of the patients after twelve weeks or less than twelve weeks of treatment.

In other embodiments, a decrease of at least about 50%, or at least about 40%, or at least about 30% in the number of inflammatory and/or non-inflammatory acne vulgaris lesions is observed in at least 65% of the patients after twelve weeks or less than twelve weeks of treatment. In one or more other embodiments, a decrease of at least about 90% at least about 70%, at least about 60% in the number of inflammatory acne vulgaris lesions is observed in at least 35% of the patients after twelve weeks or less than twelve weeks of treatment. In one or more other embodiments, a decrease of at least about 90%, at least about 70%, or at least about 60%, in the number of non-inflammatory acne vulgaris lesions is observed in at least 25% of the patients after twelve weeks or less than twelve weeks of treatment.

In other embodiments, a decrease of at least about 30% in the number of inflammatory and non-inflammatory acne vulgaris lesions is observed in at least 45% or 55% or 65% or 75% of the patients after twelve weeks or less than twelve weeks of treatment. In one or more other embodiments, a decrease of at least about 60% in the number of inflammatory acne vulgaris lesions is observed in at least 20% or 25% or 35% or 45% or 55% of the patients after twelve weeks or less than twelve weeks of treatment. In one or more other embodiments, a decrease of at least about 60%, in the number of non-inflammatory acne vulgaris lesions is observed in at in at least 20% or 25% or 35% or 45% or 55% of the patients after twelve weeks or less than twelve weeks of treatment.

In one or more embodiments, there is provided a method for treating acne vulgaris, including administering topically to a surface having acne vulgaris a composition comprising a tetracycline antibiotic, wherein after six weeks or less than six weeks of treatment, at least about 45% of the treated acne vulgaris lesions disappear (in other words, a 45% decrease in the number of lesions) so that no further antimicrobial therapy is necessary. In some embodiments, at least about 50%, at least about 60%, at least about 70% or at least about 80% of the treated acne vulgaris lesions disappear after six week or less than six weeks of treatment. In one or more embodiments, at least about 90% of the treated acne vulgaris lesions disappear after six week or less than six weeks of treatment.

In one or more embodiments there is provided a method for treating acne, including administering topically, to a surface having acne, a composition comprising a tetracycline antibiotic, wherein a percent of total number lesions that disappeared is at least about 30%, at least about 40%, or at least about 50% after three weeks or less than three weeks of treatment.

In one or more embodiments there is provided a method for treating acne, including administering topically, to a surface having acne, a composition comprising a tetracycline antibiotic, wherein a percent of total number lesions that disappeared is at least about 30%, at least about 40%, or at least about 50% after four weeks or less than four weeks of treatment.

In one or more embodiments there is provided a method for treating acne, including administering topically, to a surface having acne, a composition comprising a tetracycline antibiotic, wherein a percent of total number lesions that disappeared is at least about 30%, at least about 40%, or at least about 50% after five weeks or less than five weeks of treatment.

In one or more embodiments there is provided a method for treating acne, including administering topically, to a surface having acne, a composition comprising a tetracycline antibiotic, wherein a percent of total number lesions that disappeared is at least about 30%, at least about 40%, or at least about 50% or at least about 60%, or at least about 70%, or at least about 80% after six weeks or less than six weeks of treatment.

In one or more embodiments there is provided a method for treating acne, including administering topically, to a surface having acne, a composition comprising a tetracycline antibiotic, wherein a percent of total number lesions that disappeared is at least about 30%, at least about 40%, or at least about 50% or at least about 60% after seven weeks or less than seven weeks of treatment.

In one or more embodiments there is provided a method for treating acne, including administering topically, to a surface having acne, a composition comprising a tetracycline antibiotic, wherein a percent of total number lesions that disappeared is at least about 30%, at least about 40%, or at least about 50% or at least about 60% after eight weeks or less than eight weeks of treatment.

In one or more embodiments there is provided a method for treating acne, including administering topically, to a surface having acne, a composition comprising a tetracycline antibiotic, wherein a percent of total number lesions that disappeared is at least about 30%, at least about 40%, or at least about 50% or at least about 60%, or at least about 65%, or at least about 70%, or at least about 80% after nine weeks or less than nine weeks of treatment.

In one or more embodiments there is provided a method for treating acne, including administering topically, to a surface having acne, a composition comprising a tetracycline antibiotic, wherein a percent of total number lesions that disappeared is at least about 30%, at least about 40%, or at least about 50% or at least about 60%, or at least about 70% or at least about 75% or at least about 80% after twelve weeks or less than twelve weeks of treatment.

In one or more embodiments, there is provided a method for treating acne, including administering topically, to a surface having acne, a composition comprising a tetracycline antibiotic, wherein a percent of total number lesions that disappeared is at least about 50%, or at least about 60%, or at least about 70%, or at least about 80% after four weeks after the end of the treatment.

In one or more embodiments there is provided a method for treating acne, including administering topically, to a surface having acne, a composition comprising a tetracycline antibiotic, wherein a percent of total number lesions that disappeared at the end of treatment is statistically significant compared to baseline in both 1% and 4% dose groups.

In one or more embodiments there is provided a method for treating acne, including administering topically, to a surface having acne, a composition comprising a tetracycline antibiotic, wherein a percent of total number lesions that disappeared at the end of treatment compared to baseline is statistically significant in both 1% and 4% dose groups when compared to to placebo.

In one or more embodiments there is provided a method for treating acne, including administering topically, to a surface having acne, a composition comprising a tetracycline antibiotic, wherein a percent of total number lesions that disappeared at the end of treatment compared to baseline is statistically significant in the 4% dose group when compared to to placebo.

In one or more embodiments there is provided a method for treating acne, including administering topically, to a surface having acne, a composition comprising a tetracycline antibiotic, wherein the baseline severity of acne is at least moderate to severe, as judged by the number of acne lesions and investigator's global severity assessment (IGA).

In one or more embodiments there is provided a method for treating acne, including administering topically, to a surface having acne, a composition comprising a tetracycline antibiotic, wherein the mean number of inflammatory acne lesions at baseline is at least about 35, and the mean number of non-inflammatory lesions is at least about 45.

In one or more embodiments there is provided a method for treating acne, including administering topically, to a surface having acne, a composition comprising a tetracycline antibiotic, wherein the number of lesions is at least 20 lesions. In other embodiments there is at least one lesion, or at least 5, or at least 10 or at least 15 lesions and in further embodiments there is at least 25, or at least 30 or at least 40 or at least 50 lesions.

In one or more embodiments there is provided a method for treating acne, including administering topically, to a surface having acne, a composition comprising a tetracycline antibiotic, wherein the acne is low to moderate acne. In other embodiments the composition may be applied as aforesaid as a method of protecting the skin, for example, by preventing microbial infection or inflammatory acne

In one or more embodiments there is provided a method for treating acne, including administering topically, to a surface having acne, a composition comprising a tetracycline antibiotic, wherein the IGA score as assessed by the investigator at baseline is between 3.3-3.4, indicating moderate to severe acne at baseline. In other embodiments the composition may be applied to mild acne and in still further embodiments it may be applied to very severe acne.

In one or more embodiments there is provided a method for treating acne, including administering topically, to a surface having acne, a composition comprising a tetracycline antibiotic, wherein the daily application of topical minocycline foam (4% and 1%) on facial skin with moderate to severe acne results in a significant improvement of the disease, for example, as indicated by the primary and secondary endpoints.

In one or more embodiments there is provided a method for treating acne, including administering topically, to a surface having acne, a composition comprising a tetracycline antibiotic, wherein there is a clinically and statistically significant reduction in a lesion count selected from a group consisting of inflammatory, non-inflammatory and sum (total) acne lesions after twelve weeks of treatment in the subjects receiving minocycline foam compared to Placebo. In all 3 groups a statistically significant mean percent reduction in the number of lesions was observed.

In one or more embodiments there is provided a method for treating acne, including administering topically, to a surface having acne, a composition comprising a tetracycline antibiotic, wherein a clinically and statistically significant improvement in the investigator global assessment of acne severity after 12 treatment weeks in the subjects receiving minocycline foam compared to Placebo.

In one or more embodiments there is provided a method for treating acne, including administering topically, to a surface having acne, a composition comprising a tetracycline antibiotic, wherein the effect of the minocycline foam is dose dependent, and the effect 4% minocycline foam was generally greater than 1% minocycline foam.

In one or more embodiments there is provided a method for treating acne, including administering topically, to a surface having acne, a composition comprising a tetracycline antibiotic, wherein, a clinically and statistically significant reduction in the number of inflammatory lesions compared to baseline can be seen after 3 weeks of treatment in subjects receiving the 4% minocycline foam compared to Placebo.

In one or more embodiments there is provided a method for treating acne, including administering topically, to a surface having acne, a composition comprising a tetracycline antibiotic, wherein, clinically and statistically significant reduction in the mean percent of total lesions compared to baseline can be seen after six treatment weeks in subjects receiving the 4% minocycline foam compared to Placebo.

In one or more embodiments there is provided a method for treating acne, including administering topically, to a surface having acne, a composition comprising a tetracycline antibiotic, wherein, more than a 70% reduction in inflammatory lesion counts can be seen following 6 weeks of treatment in subjects receiving the 4% minocycline foam compared to Placebo.

In one or more embodiments there is provided a method for treating acne, including administering topically, to a surface having acne, a composition comprising a tetracycline antibiotic, wherein, the percent of subjects who had a decrease of more than 50%, 60%, 70%, or 80% in the inflammatory lesions count was statistically significantly higher in the 4% treatment group compared to Placebo after 6 treatment weeks and onward.

In one or more embodiments there is provided a method for treating acne, including administering topically, to a surface having acne, a composition comprising a tetracycline antibiotic, wherein, the percent of subjects who had a decrease of more than 50%, 60% or 70%, in the total lesions count was statistically significantly higher in the 4% treatment group compared to Placebo at twelve treatment weeks.

In one or more embodiments there is provided a method for treating acne, including administering topically, to a surface having acne, a composition comprising a tetracycline antibiotic, wherein, the percent of subjects who had a decrease of more than 50% or 70%, in the non-inflammatory lesions count was statistically significantly higher in the 4% treatment group compared to Placebo only at twelve treatment weeks.

In one or more embodiments there is provided a method for treating acne, including administering topically, to a surface having acne, a composition comprising a tetracycline antibiotic, wherein according to the investigator's global assessment at 12 weeks, more than half of the subjects have ‘clear’ or ‘almost clear’ skin in subjects receiving the 4% minocycline foam and wherein this change is statistically significant compared to subjects in the Placebo group.

In one or more embodiments there is provided a method for treating acne, including administering topically, to a surface having acne, a composition comprising a tetracycline antibiotic, wherein improvement of at least 2 grades in the investigator's global assessment score is in at least 35% of the subjects receiving 4% minocycline foam and wherein this is statistically more frequent than in subjects receiving Placebo.

In one or more embodiments there is provided a method for treating acne, including administering topically, to a surface having acne, a composition comprising a tetracycline antibiotic, wherein according to secondary endpoint relating to acne improvement assessment by the investigator after 12 treatment weeks indicated ‘excellent’ improvement in approximately two thirds of subjects in subjects receiving the 4% minocycline foam and about half of the subjects receiving the 1% minocycline foam and wherein this is statistically significant compared to the Placebo group.

In one or more embodiments there is provided a method for treating acne, including administering topically, to a surface having acne, a composition comprising a tetracycline antibiotic, wherein at least half the subjects receiving the 4% minocycline foam evaluated their acne as ‘much better than prior to study’ and wherein this is statistically significant when compared to the Placebo group.

In one or more embodiments there is provided a method for treating acne, including administering topically, to a surface having acne, a composition comprising a tetracycline antibiotic, wherein the effect was most notably shown on inflammatory acne lesions in subjects receiving the 4% minocycline foam.

In one or more embodiments there is provided a method for treating acne, including administering topically, to a surface having acne, a composition comprising a tetracycline antibiotic, wherein a percent of total number of inflammatory lesions and non-inflammatory lesions that disappeared at the end of treatment compared to baseline is higher than placebo in both 1% and 4% dose groups.

In one or more embodiments the placebo formulation has a beneficial effect. In one or more embodiments there is provided a method for treating acne, including administering topically, to a surface having acne, a placebo composition being a vehicle composition described herein for the delivery of a tetracycline that does not comprise a tetracycline antibiotic, wherein a percent of total number lesions that disappeared at the end of treatment compared to baseline is higher than on a surface having acne that is untreated. In an embodiment placebo is statistically better than no treatment.

In one or more embodiments there is provided a method for treating acne, including administering topically, to a surface having acne, a composition comprising a tetracycline antibiotic, wherein a percent of total number of inflammatory lesions that disappeared at the end of treatment compared to baseline is statistically higher than the number of non-inflammatory lesions that disappeared in both 1% and 4% dose groups.

In one or more embodiments there is provided a method for treating acne, including administering topically, to a surface having acne, a composition comprising a tetracycline antibiotic, wherein a percent of total number of inflammatory lesions that disappeared at the end of treatment compared to baseline is statistically lower than the number of non-inflammatory lesions that disappeared in both 1% and 4% dose groups.

In one or more embodiments there is provided a method for treating acne, including administering topically, to a surface having acne, a composition comprising a tetracycline antibiotic, wherein a percent of total number of lesions that disappeared in the 4% dose group at the end of treatment is significantly statistically higher than that of the 1% dose group.

In one or more embodiments there is provided a method for treating acne, including administering topically, to a surface having acne, a composition comprising a tetracycline antibiotic, wherein a percent of total number of inflammatory lesions that disappeared in the 4% dose group at the end of treatment is significantly statistically higher than that of the 1% dose group.

In one or more embodiments there is provided a method for treating acne, including administering topically, to a surface having acne, a composition comprising a tetracycline antibiotic, wherein a percent of total number of inflammatory lesions that disappeared in the 4% dose group at the end of treatment is significantly statistically higher than that of placebo.

In one or more embodiments there is provided a method for treating acne, including administering topically, to a surface having acne, a composition comprising a tetracycline antibiotic, wherein a percent of total number of inflammatory lesions that disappeared in both T % and 4% dose groups at the end of treatment is statistically significant when compared to to placebo.

In one or more embodiments there is provided a method for treating acne, including administering topically, to a surface having acne, a composition comprising a tetracycline antibiotic, wherein a percent of total number of non-inflammatory lesions that disappeared in the 4% dose group at the end of treatment is significantly statistically higher than that of the 1% dose group.

In one or more embodiments there is provided a method for treating acne, including administering topically, to a surface having acne, a composition comprising a tetracycline antibiotic, wherein a percent of total number of non-inflammatory lesions that disappeared in the 4% dose group at the end of treatment is significantly statistically higher than that of placebo.

In one or more embodiments there is provided a method for treating acne, including administering topically, to a surface having acne, a composition comprising a tetracycline antibiotic, wherein a percent of total number of non-inflammatory lesions that disappeared in both T % and 4% dose groups at the end of treatment is statistically significant when compared to to placebo.

In one or more embodiments there is provided a method for treating acne, including administering topically, to a surface having acne, a composition comprising a tetracycline antibiotic, wherein at least about 40%, or at least about 50%, or at least about 60%, or at least about 62%, or at least about 64%, or at least about 66%, or at least about 68%, or at least about 70%, or at least about 75% of total number lesions disappear after four weeks after the end of the treatment (F/U). In one or more embodiments these changes at F/U are statistically significant compared to baseline in both 1% and 4% dose groups. In one or more embodiments these changes at F/U are statistically significant compared to placebo in both T % and 4% dose groups. In one or more embodiments the number of lesions at F/U is the same or similar compared to EOT in both 1% and 4% dose groups. In one or more embodiments the number of lesions at F/U increases compared to EOT. In one or more embodiments there is the number of lesions at F/U decreases compared to EOT.

It is postulated, without being bound by any theory, that the use of a hydrophobic oil based foam vehicle contributes to cutaneous bioavailability, including the achievement of therapeutic levels of minocycline in the pilosebaceous unit. Specific targeting of hydrophobic oil based foam vehicle to the pilosebaceous unit is enabled due the hydrophobic nature of the pilosebaceous gland.

In-vitro skin penetration studies (see e.g. WO11/039637) show that topical administration of minocycline brings appreciable amounts of the drug to its target site of action—the skin, thus it possible to avoid the undesirable high systemic exposure and the negative consequences of the oral dosage route.

Ex-vivo studies surprisingly revealed that minocycline successfully penetrates the skin and hair follicle and targets the sebaceous gland area. (see Example 4).

In one or more embodiments, the methods for treating acne, as set out herein, are able to deliver effective amounts of a tetracycline antibiotic into the skin or mucosal surface.

In one or more embodiments, the methods for treating acne, as set out herein, are able to deliver effective amounts of a tetracycline antibiotic into and around the hair follicle or the hair follicle area.

In one or more embodiments, the methods for treating acne, as set out herein, are able to deliver effective amounts of a tetracycline antibiotic into or around the sebaceous gland or the sebaceous gland area or the pilosebaceous unit.

In one or more embodiments, the methods for treating acne, as set out herein, are able to deliver effective amounts of a minocycline, wherein the minocycline composition targets the sebaceous gland or the sebaceous gland area or the pilosebaceous unit.

In one or more embodiments, there is provided a method for treating acne, as set out herein, wherein the hydrophobic gel or foam composition targets the hair follicle or the hair follicle area.

In one or more embodiments, there is provided a vehicle for delivering a therapeutically effective amount of active agent to the sebaceous gland or the sebaceous gland area or the pilosebaceous unit comprising:
a) about 60% to about 99% by weight of at least one hydrophobic solvent;
b) at least one viscosity-modifying agent comprising a wax and a fatty alcohol or a fatty acid, or both.

In one or more embodiments, there is provided a vehicle for delivering a therapeutically effective amount of active agent to the sebaceous gland or the sebaceous gland area or the pilosebaceous unit comprising:
b) about 60% to about 99% by weight of at least one hydrophobic solvent;
c) at least one viscosity-modifying agent comprising a wax and a fatty alcohol or a fatty acid, or both;
wherein the active agent is a tetracycline antibiotic.

In one or more embodiments, there is provided a vehicle for delivering a therapeutically effective amount of active agent to the sebaceous gland or the sebaceous gland area or the pilosebaceous unit comprising:
d) about 60% to about 99% by weight of at least one hydrophobic solvent;
e) at least one viscosity-modifying agent comprising a wax and a fatty alcohol or a fatty acid, or both;
wherein the active agent is a minocycline.

In one or more embodiments, there is provided an effective method for treating acne, as set out herein to patients with more than twenty inflammatory but not more than fifty inflammatory lesions on the face (papules and/or pustules), with more than twenty five but not more than a hundred non-inflammatory lesions on the face (opened and/or closed comedones), no significant nodulocystic acne vulgarison the face (less than 2 lesions) and receiving a score of at least Moderate on the Investigator's Global Assessment Scale.

The clinical studies affirmed that the once-daily treatment regimen with minocycline foam (1% or 4%) was safe even for a prolonged treatment period. During twelve weeks of treatment no serious adverse events were reported, no occurrences of pigmentation or itching were noted and only a few occurrences of erythema, dryness and peeling were recorded in acne vulgaris patients. This is advantageous as most approved topical prescription treatments currently available to treat acne vulgaris are in the form of creams and ointments and are associated with skin irritation such as pigmentation, erythema, dryness, peeling and itching which impede patient compliance. The present foamable compositions meet the long felt need for a convenient safe method of treatment of acne which minimizes or avoids skin irritation and avoids serious adverse events associated with other treatments including oral tetracyclines even when applied for a prolonged period while maintaining efficacy Thus, Minocycline topical foam administered once daily was efficient, safe and well-tolerated.

Most approved topical prescription treatments currently available to treat acne vulgaris require a twelve week treatment regimen, which may impact patient compliance. In contrast, the present gel, liquid gel and foamable compositions meet a long felt need for a shorter treatment regimen having an earlier onset and a higher percentage reduction in lesions, while maintaining high levels of safety and efficacy.

As with other therapeutic regimens, patient compliance is essential in the effectiveness of prescribed antibiotics. With poor compliance, therapeutic goals are less likely to be achieved, resulting in poorer patient outcomes. Poor compliance is associated with deteriorating skin condition, the need for additional consultations, the emergence of bacterial resistance, extra drugs, additional expenses on cosmeticians and increases in direct and indirect costs of healthcare management.

In general, patients are more compliant with simple and shorter dosing regimens. Both the dosage schedule and the patient's daily routine should be considered when prescribing antibiotics. Topical agents may also be more attractive than oral therapy because they reduce the potential for systemic side effects, typically nausea and diarrhea, which are commonly associated with many systemic antibiotics. They may also help provide a reduction in cross contamination by providing a barrier with antibiotic over the infected area.

In one or more embodiments there is provided a method for treating acne, including administering topically, to a surface having acne, a composition comprising a tetracycline antibiotic administered at least alternate days or once daily which has a high or improved patient compliance compared with existing treatments.

In one or more embodiments one or more of the methods herein of treating or alleviating acne or acne vulgaris can also be used for treating a disorder including one or more of the following: acne related or associated disorder, acne like symptoms, acne related symptoms, a tetracycline antibiotic responsive acne related disorder, skin disorder caused by a bacteria, and a tetracycline antibiotic responsive sebaceous gland disease, P. acne bacteria associated disorders and other superficial infections, including skin infections.

In one or more embodiments there is provided a method of maintenance therapy, to prevent acne recurrence or reduce the severity of the acne recurrence, applied to a patient in need which comprises applying to the skin on a regular basis (as defined above) a hydrophobic gel or foam composition comprising a therapeutically effective amount of a tetracycline antibiotic

In one or more embodiments there is provided a regime or regimen for treating a patient having one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections which comprises applying to the afflicted area on a regular basis a hydrophobic gel or foam composition, said composition comprising a therapeutically effective amount of a tetracycline antibiotic.

In one or more embodiments there is provided the use of an tetracycline antibiotic compound for the manufacture of a medicament for treating one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections in a human subject in need thereof, wherein the tetracycline antibiotic compound is to be administered topically to said human subject in an amount that is effective to treat acne.

In one or more embodiments there is provided a tetracycline antibiotic compound for use as a medicament in treating and/or preventing one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders, and other superficial infections, including skin infections wherein the tetracycline antibiotic is used in a hydrophobic gel or foam composition administered topically at least alternate days or at least once daily for at least six weeks.

In one or more embodiments there is provided a hydrophobic gel or foam composition comprising a tetracycline antibiotic for use in treating acne in a human subject suffering therefrom comprising topically administering the composition to the human subject in a sufficient amount and for a sufficient time to decrease the number of acne lesions by at least 25%.

In one or more embodiments there is provided a hydrophobic gel or foam composition comprising a tetracycline antibiotic for use in treating one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections, wherein the hydrophobic gel or foam composition is administered topically at least alternate days or at least once daily for twelve weeks or less than twelve weeks of treatment.

In one or more embodiments there is provided a hydrophobic gel or foam composition comprising a therapeutically effective amount of tetracycline antibiotic for use in treating acne in a human subject comprising topically administering the composition at least alternate days or at least once daily, wherein a decrease the number of acne lesions is observed after at least six weeks of treatment.

In one or more embodiments there is provided a hydrophobic gel or foam composition comprising a therapeutically effective amount of tetracycline antibiotic for use in treating acne in a human subject comprising topically administering the composition at least alternate days or at least once daily, wherein a decrease the total number of acne lesions is observed after at least three weeks of treatment.

In one or more embodiments there is provided a hydrophobic gel or foam composition comprising a therapeutically effective amount of tetracycline antibiotic for use in treating acne in a human subject comprising topically administering the composition at least alternate days or at least once daily, wherein a decrease the number of inflammatory acne lesions is observed after at least three weeks of treatment.

In one or more embodiments there is provided a hydrophobic gel or foam composition comprising a minocycline antibiotic for use in treating a disorder selected from the group consisting of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections, wherein the hydrophobic gel or foam composition is administered topically at least alternate days or at least once daily for at least six weeks to the skin, wherein the hydrophobic gel or foam composition is waterless and does not comprise a silicone other than cyclomethicone.

In one or more embodiments there is provided a hydrophobic gel or foam composition comprising a minocycline antibiotic for use in treating a disorder selected from the group consisting of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections, wherein the hydrophobic gel or foam composition is administered topically at least alternate days or at least once daily for at least six weeks to the skin, mucosa, or eye, wherein the hydrophobic gel or foam composition is waterless and does not comprise a polyethylene gelling agent or polyethylene homopolymer or polyethylene copolymer.

In one or more embodiments there is provided a hydrophobic gel or foam composition comprising a minocycline antibiotic for use in treating a disorder selected from the group consisting of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections, wherein the hydrophobic gel or foam composition is administered topically at least alternate days or at least once daily for at least six weeks to the skin, wherein the minocycline antibiotic is the sole active ingredient present in the composition. In one or more embodiments there is provided a hydrophobic foam composition or gel comprising a tetracycline antibiotic for use in retarding, arresting, or reversing the progression of one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections, wherein the hydrophobic foam composition or gel is applied topically to the skin at least alternate days or at least once a day for at least six weeks.

In one or more embodiments the human subject is 30 or less than 30 years old, or is 25 or less than 25 years old, or is 22 or is less than 22 years old, or is 20 or less than 20 years old, or is 18 or less than 18 years old, or 15 or is less than 15 years old, or is between 8 to 25 years old or is between 9 to 22 years old. In an embodiment the subject is a female. In an embodiment the female is under the age of forty six and optionally is a pregnant or breastfeeding female. In an embodiment the subject is a male. In an embodiment the subject is a teenager. In another embodiment the subject is a child.

Thus, it was unexpectedly demonstrated that topical minocycline foam offered a safe and effective alternative to topical compositions containing for example retinoids and BPO for the topical treatment of acne vulgaris. The ease of use, with once daily dosing, as well as its broad spectrum of activity, early onset, the low level of adverse events and the rapid reduction in the number of lesions make it an attractive choice and a potentially valuable medication for the treatment of acute bacterial skin infections.

Further provided herein is a method of treating human skin disorders such as acne, rosacea, and/or impetigo by topical application of a foam or gel or liquid gel as described herein to a patient in need thereof.

The invention is described with reference to the following examples, in a non-limiting manner. The following examples exemplify the foamable compositions and methods described herein. The examples are for the purposes of illustration only and are not intended to be limiting. Many variations will suggest themselves and are within the full intended scope.

### Methods

### Canisters Filling and Crimping

Each aerosol canister is filled with the pre-foam formulation (“PFF”, i.e., foamable carrier) and crimped with valve using vacuum crimping machine. The process of applying a vacuum will cause most of the oxygen present to be eliminated. Addition of hydrocarbon propellant may, without being bound by any theory, further help to reduce the likelihood of any remaining oxygen reacting with the active ingredient. It may do so, without being bound by any theory, by one or more of dissolving in, to the extent present, the oil or hydrophobic phase of the formulation, by competing with some oxygen from the formulation, by diluting out any oxygen, by a tendency of oxygen to occupy the dead space, and by oxygen occupying part of the space created by the vacuum being the unfilled volume of the canister or that remaining oxygen is rendered substantially ineffective in the formulation.

### Pressurizing & Propellant Filling

Pressurizing is carried out using a hydrocarbon gas or gas mixture. Canisters are filled and then warmed for 30 seconds in a warm bath at 50° C. and well shaken immediately thereafter.

### Tests

By way of non-limiting example the objectives are briefly set out below as would be appreciated by a person of skill in the art.

### Collapse Time

Collapse Time, which is the measure of thermal stability, is examined by dispensing a given quantity of foam and photographing sequentially its appearance with time during incubation at 36° C. The collapse time result is defined as the time when the foam height reaches 50% of its initial height or if the foam has not yet reached 50% of its initial height after say 180 seconds then the collapse time is recorded as being >180. By way of illustration, one foam may remain at 100% of its initial height for three minutes, a second foam may reach 90% of its initial height after three minutes, a third foam may reach 70% of its initial height after three minutes, and a fourth foam may reach 51% of its initial height after three minutes, nevertheless in each of these four cases the collapse time is recorded as >180 seconds since for practical purposes for easy application by a patient to a target the majority of the foam remains intact for more than 180 seconds. If the foam, for example, reaches 50% of its original height after say 100 seconds it would be recorded as having a collapse time of 100 seconds. It is useful for evaluating foam products, which maintain structural stability at skin temperature for at least 1 minute. Foams which are structurally stable on the skin for at least one minute are termed “short term stable” carriers or foams.

Alternatively, a Simple Collapse Time can be assessed by placing a foam sample on the warm fingers of a volunteer and measuring the time it takes to melt on the fingers.

### Viscosity

Viscosity is measured with Brookfield LVDV-II+PRO with spindle SC4-25 at ambient temperature and 10, 5 and 1 RPM. Viscosity is usually measured at 10 RPM. However, at about the apparent upper limit for the spindle of ˜>50,000 CP, the viscosity at 1 RPM may be measured, although the figures are of a higher magnitude. Unless otherwise stated, viscosity of the pre-foam formulation (PFF) is provided. It is not practical to try and measure the viscosity of the foamable formulation with regular propellants since they have to be stored in sealed pressurized canisters or bottles. In order to simulate the viscosity in the foamable formulations with propellant an equivalent weight of pentane (a low volatile hydrocarbon) is added to and mixed with the pre-foam formulation and left overnight. The viscosity is then measured as above.

### FTC (Freeze Thaw Cycles)

Foam appearance under extreme conditions of repeated heating and cooling is evaluated by cycling through cooling, heating, (first cycle) cooling, heating (second cycle) etc., conditions, commencing with −10° C. (24 hours) followed by +40° C. (24 hours) and measuring the appearance following each cycle. The cycle is repeated up to three times.

### Chemical Stability

The amount of active agent present is analyzed chromatographically in foam released from various pressurized canisters or in the gel or liquid gel. Analysis is carried out at baseline and at appropriate time intervals thereafter. The canisters are typically stored in controlled temperature incubators at one or more of 5° C., 25° C., 40° C. and 50° C. At appropriate time intervals canisters are removed and the amount of active agent in the foam sample is measured.

### Microbiological Tests

Microbial load: Testing was performed according to EP 2.6.12 and 2.6.13 as described in the European Pharmacopeia.

Preservative efficacy: Testing was performed according to USP <51> and EP 5.6, 2007 5.1.3. as described in the European and US Pharmacopeia.

The test consists of challenging the product with specified microorganisms, storing the inoculated preparations at a prescribed temperature, removing the inoculated samples at specified intervals of time and counting the number of viable organisms in the withdrawn samples using a plate-count procedure. Formulations were challenged by introducing the following microorganisms:
Escherichia co/i (ATCC no. 8739)
Staphylococcus aureus (ATCC no. 6538)
Pseudomonas aeruginosa (ATCC no. 9027)
Candida albicans (ATCC no. 10231)
Aspergillus niger (ATCC no. 16404)

The number of colony-forming units (cfu/g) determined at each incubation time point was compared to the number of cfu/g measured in non-inoculated control samples. In order to verify that the samples tested are free of microbial contaminants, the microbial load (base-line) in the samples was determined prior to preservative efficacy testing. Study results are expressed as the number of surviving microorganisms (cfu/g).

Water Activity (Aw): The test for water activity was performed on pre-foam formulation samples introduced into the measuring cell of a PAWKIT water activity meter from DECAGON.

In-vitro effect on microbial growth: The tested microorganism is grown on Tryptic Soy Agar Slants. After incubation, the bacteria is harvested using sterile buffer phosphate pH 7.0, to obtain a microbial count of about 10⁴ cfu/ml. 0.2 ml of the above suspension is spread on Letheen Agar plate and put aside to dry for 20 minutes at room temperature. A sterile disc of 6 mm diameter which has been soaked in 10 μl of the tested antibacterial pre-foam-formulation (PFF) is put on the microbial film, the plate is incubated at 35° C. for 1-2 days. A control experiment is also performed where no antibacterial material is put on the sterile discs. Antimicrobial activity of the tested material inhibits growth of the microorganism around the disc, leaving a transparent zone around it. The diameter of the inhibition zone is measured in mms.

### Compatibility

Active agent is incubated with various excipients individually at one or more temperatures and at different ratios of active agent to a single excipient for a certain fixed period or to the point where degradation was suspected. The period can be for example 3 or 7 or 14 or 21 or 28 days or longer. Visual inspection is a criterion for indication of compatibility. Any change of color indicates oxidation or degradation. For example, the color of an intact MCH suspension is a pale yellow; and a change of color e.g., to dark orange, red, green, brown and black, indicates oxidation or degradation. Tests are also carried out with combinations of excipients.

### Ex-Vivo Studies

Two mg/cm² of the minocycline foam formulation, according to COLIPA and United States standards, were applied on pig ear skin. A skin area of 10×8 cm² was marked with a permanent marker. 160 mg of the formulation was applied with a syringe and distributed homogeneously with a gloved finger. Subsequent to application the substance was left on the skin for an hour. A biopsy was taken upon completion of penetration period. Histological sections of the hair follicles were prepared subsequent to biopsy. Pictures were obtained using fluorescence microscopy, reflectance and fluorescent images being superimposed.

### EXAMPLES

### Example 1—General Manufacturing Procedures for a Gel or a Foam

The following procedures were used to produce gel or foam samples, in which only the steps relevant to each formulation were performed depending on the type and nature of ingredients used.

Step 1: Hydrophobic solvents such as soybean oil, coconut oil, mineral oils are mixed at room temperature.

Step 2: The formulation is warmed to 85° C. and solid compounds such as fatty alcohols, fatty acids and waxes are added and mixed until complete dissolution. Fumed silica and if present color agents are also added and mixed at this stage.

Step 3: The formulation is cooled down to 30-40° C., cyclomethicone and active agents such as tetracyclines are added under mixing until formulation homogeneity is obtained.

Step 4: For gel compositions, the formulation is packaged in suitable containers. For foamable compositions, the formulation is packaged in aerosol canisters which are crimped with a valve, pressurized with propellant and equipped with an actuator suitable for foam dispensing. Optionally, a metered dosage unit can is utilized, to achieved delivery of desirable and/or repeatable measured doses of foam.

Step 5: For foamable compositions, pressurizing is carried out using a hydrocarbon gas or gas mixture. Canisters are filled and then warmed for 2 minutes in a warm bath at 60° C. and well shaken immediately thereafter.

Step 6: The canisters or containers are labeled.

### Example 2—Preliminary Study in an Acne Vulgaris Patient

### 1. Study Synopsis

STUDY TITLE: A preliminary study, to assess the efficacy safety and tolerability of topically applied FXFM244 antibiotic foam in the treatment of acne vulgaris.

Objectives: To assess the efficacy, safety and tolerability of topical application of FXFM244 antibiotic foam in a human subject having acne vulgaris.

STUDY MEDICATION: Minocycline hydrochloride foam (1% FXFM244 antibiotic foam), as described in section 10 below.

DOSAGE: Subject's forehead was treated topically once daily at bedtime for 6 weeks.

INDICATION: Acne vulgaris.

DESIGN: The forehead of a fourteen year old male subject having acne vulgaris was cleansed, then treated topically with 1% FXFM244 antibiotic foam at bedtime once daily for 6 weeks. Subject's forehead was photographed at baseline and after 6 weeks of treatment.

VARIABLES: Efficacy, safety and tolerability in the treatment of acne vulgaris.

### 2. Study Results

Following six weeks of treatment, in which the foamable composition containing 1% minocycline was applied once daily on the forehead of a fourteen year old male afflicted with acne vulgaris, an unexpected decrease in the number of both inflammatory and non-inflammatory lesions and a significant improvement in the skin condition was observed (See FIG. 1). As shown in FIG. 1, the improvement is apparent as is also the restoration of visible, normal cutaneous topographic features, indicating the return of skin integrity. No systemic adverse events and no dermal adverse events, e.g., skin irritation, photosensitization, pigmentation, erythema, dryness, itching or peeling normally associated with oral antibiotics or other available topical formulation were observed.

The lack of pigmentation and photosensitization is surprising as hyper-pigmentation and photosensitization of the skin are known dermal side effects of oral minocycline. It could therefore be that more severe pigmentation and photosensitization might be observed if minocycline is applied directly onto the skin; but this was not the case. FIG. 1 demonstrates nearly complete clearance of both types of lesions in the treated area. In comparison oral minocycline is said to achieve about 50% clearance of inflammatory lesions and no effect on non-inflammatory lesions. These preliminary results may suggest that the topical compositions of the present invention can be as effective as, or more effective than, the alternatives without untoward reactions.

### Example 3—Clinical Study Phase II in Acne Vulgaris Patients

### 1. Study Synopsis

STUDY TITLE: A Pilot, Multicenter, Randomized, Double Blind, Placebo Controlled, Parallel Group, Dose Range Finding Study, to Evaluate the Tolerability and Safety of FXFM244 Antibiotic Foam and to Monitor its Clinical Effect in Acne Vulgaris Patients.

OBJECTIVES: (i) To assess the safety and tolerability of topical FXFM244 antibiotic foam in a population of acne vulgaris patients; (ii) To detect clinically significant efficacy of FXFM244 antibiotic foam for treatment of acne vulgaris and (iii) to establish a safe and effective dose.

STUDY MEDICATION: Minocycline hydrochloride foam (MCH 1% and MCH 4% compositions, as described below) and placebo (vehicle foam) as set out in Table 10. below.

DOSAGE: Patients were treated topically on the facial skin areas affected by acne vulgaris once daily for 12 weeks. The first dose was applied in the presence of the study Investigator or his assignee. Subsequent applications were made by the patient once daily at bedtime to the same region.

INDICATION: Acne vulgaris

DESIGN: 150 male or female patients, between ages of 12 to 25 years, diagnosed with moderate to severe acne vulgaris with facial involvement entered a randomized, double (investigator/patient) blind, placebo controlled, parallel group, dose range finding study. At the screening/baseline visit, after signing informed consent and undergoing screening procedures, eligible subjects were randomized in 1:1:1 ratio to one of three treatment groups receiving, 1% minocyclie foam FXFM244, 4% minocyclie foam FXFM244 or Placebo. The first dose of study drug was applied topically in the presence of study investigator or designee to facial skin areas. Subsequently, subjects continued to self-apply study drug to the same region daily, in the evening, for 12 weeks. Subjects returned to the clinic for efficacy, safety and tolerability evaluations 3, 6, 9 and 12 weeks post baseline visit. A post-treatment follow-up visit took place 4 weeks after end of treatment.

Subjects received clear instructions on how to apply the study drug on the face area. During the 12 weeks treatment period, prior to each daily topical administration, subjects were instructed to cleanse the face area with mild or soapless, non-medicated cleanser, and not use any other acne medications or moisturizers. At each study visit, subjects were dispensed one aerosol container and were instructed to return it at the following visit to evaluate subject's compliance.

Patients were instructed to shake the can before use, dispense a small amount of foam and apply a thin layer of medication on the affected area once a day for 12 weeks. A post-treatment follow-up visit took place 4 weeks after end of treatment.

The inclusion criteria specified that patients should have (i) at least 20 but not more than 50 inflammatory lesions on the face (papules and/or pustules,); (ii) at least 25 and not more than 100 non-inflammatory lesions on the face (open and/or closed comedones); (iii) no significant nodulocystic acne on the face (≤2 lesions); (iv) a score of >3 (moderate) on the investigator's global assessment scale.

VARIABLES: Clinical examination, safety, tolerability and efficacy parameters (lesion count, global assessment, global improvement assessment, patient global improvement assessment photographs). ENDPOINTS AND OUTCOMES

### Efficacy Outcomes

1. Lesion count, number, numerical change in lesion count and % change in lesion count (inflammatory, non-inflammatory and total) at visit 2 (three weeks), 3 (six weeks), 4 (nine weeks), 5 (twelve weeks), 6 (four weeks after end of treatment (EOT)) compared to baseline;
2. % of subjects who had a decrease of more than 50%, more than 60%, more than 70% or more than 80% in lesions by study group at visit 2, 3, 4, 5, 6 compared to baseline;
a. 3. Investigator Global Assessment (IGA) for acne severity:IGA at visits 2, 3, 4, 5 and 6.
b. The statistical difference in IGA at visits 2, 3, 4, 5 and 6.
c. Number and % of subjects that meet the success criterion of “clear” or “almost clear” (grades less 2) at visits 2, 3, 4, 5, and 6
d. Number and % of subjects that meet the success criterion of “improvement of 2 grades from the baseline” at visits 2, 3, 4, 5 and 6
4. Investigator's Global Improvement Assessment (aided by photographs) comparison from baseline to visit 5 (12 weeks of treatment);
5. Patient's Global Improvement Assessment comparison from baseline to visit 5 (12 weeks of treatment)

### Safety Outcomes

Assessment of safety was conducted at all six visits using the following parameters Adverse events (AEs), physical examination, vital signs (blood pressure, heart rate, and oral body temperature) and concomitant medications.
Tolerability Outcome Clinical assessment of skin irritation (erythema, dryness, pigmentation, peeling and itching) using a scale of 0 to 3

### Statistical Methods:

All tests applied were two-tailed, and a p-value of 5% or less was considered statistically significant. The data were analyzed using STATA version 12.0 for Windows. See further discussion in section 4 below (statistical methodology).

### 2. Clinical Study Design

The protocol and informed consent forms were approved by each clinical site's local Ethics Committee (EC) and the Israel Ministry of Health prior to study initiation. To be eligible for the study, the subject or the subject's parent or legal guardian were required to sign a written informed consent document and were willing and able to comply with the requirements of the protocol.

The exclusion criteria specified that subjects had to be between ages of 12 to 25 with clinical diagnosis of moderate to severe acne vulgaris with facial involvement having the main severity criteria consisting of.
(i) at least twenty but not more than fifty inflammatory lesions on the face (papules and/or pustules) (ii) at least twenty five but not more than 100 non-inflammatory lesions on the face (open blackhead and/or closed comedones (whitehead) (iii) no significant nodulocystic acne vulgaris on the face (less than 2 lesions) and (iv) receiving a score of at least Moderate on the Investigator's Global Assessment Scale.

Subjects were required not to have had any known medical conditions that, in the Investigator's opinion could interfere with study participation. Subject had to refrain from use of all other topical acne medications or antibiotics or moisturizers, new brands of make-up, creams, lotions, powders or any topical product other than the assigned treatment to the treatment area for the duration of the study. Women of childbearing potential, had to use an acceptable form of birth control during the study. Use of oral contraceptives had to remain constant within 3 month prior to baseline and throughout the study.

The exclusion criteria specified that patients should have any one of the following (i) a disease selected form a group consisting Acne Conglobata, Acne Fulminas, secondary acne (chloracne, drug induced acne) or severe acne requiring systemic treatment; (ii) Presence of any facial skin condition that would interfere with the diagnosis or assessment of acne vulgaris (e.g. rosacea, dermatitis, psoriasis, squamos cell carcinoma, eczema, acneform eruptions caused by medications, steroid acne, steroid folliculitis, or bacterial folliculitis); (iii) Excessive facial hair (beards, sideburns, moustaches, etc.) that would interfere with diagnosis or assessment of acne vulgaris. (iii) Known or suspected hypersensitivity to minocycline or any of the excipients in the study drug. (iv) Use of concomitant medication prior to the study including:
f) Use within 6 month prior to baseline of topical retinoids, oral retinoids (Accutane®) or therapeutic vitamin A supplements of greater than 10,000 units/day (multivitamins are allowed).
g) Use of systemic steroids, systemic antibiotics, systemic treatment for Acne Vulgaris, systemic anti-inflammatory agents within 4 weeks prior to baseline.
h) Use of topical steroids, α-hydroxy/glycolic acid, benzoyl peroxide, topical antibiotics, topical treatment for Acne Vulgaris, topical anti-inflammatory agents within 2 weeks prior to baseline.
i) Use for less than 3 month prior to baseline of estrogens or change in oral contraceptives therapy within less than 3 month prior to baseline;
j) Use on the face of: cryodestruction or chemodestruction, dermabrasion, photodynamic therapy, acne surgery, intralesional steroids or X-ray therapy within 4 weeks prior to baseline.
k) Use of the following concomitant medications throughout the duration of the study (unless approved by the Investigator and medical monitor): topical antibiotics on the face, oral antibiotics, topical steroids on the face, oral steroids, topical anti-inflammatory drugs on the face, oral anti-inflammatory drugs, topical and/or oral drugs for Acne Vulgaris other than the study drug, topical retinoid drugs on the face, oral retinoid drugs or therapeutic vitamin A supplements of greater than 10,000 units/day, topical a hydroxyl/glycolic acid and/or benzoyl peroxide on the face, topical and/or Oral estrogens or any new oral contraceptives, spironolactone.
(v) Alcohol or drug abuse, according to assessment by the investigator; (vi) Known or suspected hypersensitivity to minocycline or any of the excipients in the Study Medication; (vii) Use of another investigational drug within 30 days prior baseline. (viii) Pregnant or lactating women; (ix) Use of tanning booths, sunbathing, or excessive exposure to the sun should be prohibited during the study; (x) Participation in a clinical trial in the previous month prior to randomization.

Subjects were free to discontinue their participation in the study at any time and without prejudice to further treatment. The Investigator had to discontinue participation of any subject who requested to be withdrawn, or if it was determined that continuing in the study would result in a significant safety risk to the subject. The subject's participation in this study could have been discontinued due to any of the following reasons: interrupted therapy for >5 days, worsening of the disease, occurrence of a concomitant disease, intolerable adverse event, subject withdrew consent, relevant non-compliance with the protocol, investigator decided that withdrawal from the study was in the best interest of the subject, pregnancy, subject needed or used medication not permitted under the protocol,

Treatment was administered topically on facial skin areas affected with acne vulgaris once daily (OID) at bedtime for 12 weeks. The mode of application was demonstrated by the investigator or study nurse at Visit 1 using a placebo from a demonstration kit that was supplied by the Applicants. Subjects were instructed to cleanse their face with a mild or soapless, non-medicated and then pat it dry. They were instructed to shake the canister before use, dispense a small amount of foam to a disposable plate and then apply a small amount of the foam using the tip of their finger for each area: forehead cheeks and chin. Subjects were instructed to treat the nose area only if affected and to apply the foam on the whole area, not just on visible lesions. Application was attained by collapsing and spreading it as a thin layer on the affected area. Patients were further instructed not to apply moisturizers, new brands of make-up, creams, lotions, powders or any topical product other than the assigned treatment to the treatment area. Patients were instructed to minimize exposure to sunlight, including sunlamps, while using the compositions. Use of sunscreen products over treated areas was recommended when sun exposure could not be avoided.

A total of a hundred and fifty eligible subjects (conforming to both inclusion and exclusion criteria) were enrolled and randomized into three parallel study groups, testing the two different strengths (minocycline 1% and 4%) of the study medication and placebo formulation (without minocycline) with fifty subjects in each treatment group. The study included six scheduled study visits: Day 1 (Visit 1—Baseline) screening and treatment initiation; Week 3 (±7)—(Visit 2 first interim visit); Week 6 (+7)—(Visit 3—second interim visit), Week 9 (+7)—(Visit 4—third interim visit), Week 12 (+5)—(Visit 5—End of Treatment (EOT)) and Week 16 (+7) (Visit 6—Follow-up (F/U)). Clinical assessments, efficacy, safety and tolerability evaluations were done at Baseline, interim visits, EOT and F/U. At each visit, patients were evaluated by Investigator via lesion counts, clinical assessment of skin irritation, and a global assessment and improvement assessment. Any clinical adverse events were recorded. At Week 12, in addition to the above, the patient provided his/her Patient's Global Improvement Assessment and a satisfaction questionnaire regarding usability and treatment satisfaction. (See Table 2.1 below).

TABLE 2.1
Study assessment table

| | | | | | | Follow- |
|---|---|---|---|---|---|---|
| | Treatment Period | | | | | Up |
| Study Assessment | Screening/Baseline | | | | | Period |
| Visit No. | 1 | 2 | 3 | 4 | 5 | 6 |
| Study Week | 0 | 3 | 6 | 9 | 12 | 16 |
| Visit window | 1 | ±7 | ±7 | ±7 | ±5 | ±7 |
| Informed consent | X | | | | | |
| Inclusion/exclusion criteria | X | | | | | |
| Demographic data | X | | | | | |
| Medical history | X | | | | | |
| Acne disease history | X | | | | | |
| Previous acne medications and | X | | | | | |
| response to these medications | | | | | | |
| Randomization | X | | | | | |
| Physical examination and vital signs | X¹ | X | X | X | X | X |
| Clinical assessment (skin lesion count) | X | X | X | X | X | X |
| Investigator’s Global Assessment | X | X | X | X | X | X |
| (IGA) of acne severity | | | | | | |
| Investigator’s Global Improvement | | X | X | X | X | X |
| Assessment | | | | | | |
| Photography of face area | X | X | X | X | X | X |
| Patient’s Global Improvement | | | | | X | |
| Assessment | | | | | | |
| Tolerability (skin irritation) | X | X | X | X | X | X |
| assessment | | | | | | |
| Urine pregnancy test (for | X | X | | | | |
| childbearing potential women)² | | | | | | |
| Adverse events | | X | X | X | X | X |
| Concomitant medication | X | X | X | X | X | X |
| Dispense study medication | X | X | X | X | | |
| Drug accountability and compliance | | X | X | X | X | |
| Dispense patient diary | X | | | | | |
| Collect patient diary | | | | | X | |

¹At screening/baseline, physical examination included height and weight, in addition to heart/lung and abdomen; vital signs included heart rate, blood pressure and temperature
²In case pregnancy was suspected during the study, urine pregnancy test was performed

Subject compliance was monitored using a treatment diary and was calculated as the percentage of number of days the subject actually administrated the study drug to the sum of days of study drug administration plus days with no study drug administration.

Drug compliance of patients (i.e., the amount of drug used by each subject) was calculated by weighing each container before and after use, and calculating the difference in the weight of containers before and after use, divided by the total number of days study drugs was used by each patient.

Efficacy was determined by the investigator at each visit by evaluating the change in the number of lesion (inflammatory/non inflammatory and total), % change in lesion count and Investigator's global assessment (IGA) for acne severity (assisted by scale). A global improvement assessment by the Investigator (assisted by photographs) and global improvement assessment by the patient which is a subjective assessment.

Tolerability was determined at each visit by assessment of skin irritation parameters. Safety was determined by assessment of adverse events, performing physical examination and checking vital signs at each visit, checking pregnancy potential and concomitant medications.

### 3. Patient Demographics

Patients who enrolled into the study were classified as follow:
Safety population (SAF) included all subjects who were randomized, dispensed study medications and attended at least one post-baseline visit with safety evaluation
The intent-to-treat (ITT) population consists of all randomized patients who dispensed study medications and attended at least one post-baseline visit with efficacy evaluation
The per protocol (PP) population includes all patients who completed 12 weeks of treatment with no major protocol deviations.

Modified per protocol (mPP) population included all PP population who have used a mean>0.08 g of study drug per day. The mPP population differs from the PP population by only one patient and the results of the statistical efficacy analysis of the PP and mPP are nearly the same.

The baseline demographic variables (age, gender, weight, height and BMI) of all subjects (ITT population) were comparable across the three study groups (1%, 4% minocycline foam and Placebo) (p>0.2). Overall average age was approximately 16.5 (range 12.3 to 25.0) and half of all participating subjects were men. Mean BMI was within normal range in all study groups (range 13.7 to 37.5).

Approximately one-sixth of the subjects in the ITT population had medical history events. Some had active diagnoses and others were receiving treatment for their condition. None had dermatological abnormalities other than acne. There were no notable differences between treatment groups.

All subjects with childbearing potential had a negative pregnancy test at Screening/Baseline.

All subjects had a history of acne vulgaris. In the ITT population the duration of acne vulgaris disease at time of enrollment ranged from 2-120 months, averaging approximately 35 months. There was no significant difference between the three study groups.

Study treatment compliance was evaluated in the PP population, in terms of subject compliance and drug compliance throughout the 12 weeks treatment period. There were no significant differences in subject compliance between study groups (p>0.40).

In the ITT population, the baseline severity of acne vulgaris was significantly higher than the minimum criteria. The mean lesion counts at baseline was about 34.5 (36.5, 33.5 and 33.6 in the 1%, 4% and Placebo groups, respectively) for inflammatory lesions (eligibility criterion: 20); and about 44.8 (46.2, 43.3, and 45.1 in 1%, 4%, and Placebo groups, respectively) for non-inflammatory lesions (eligibility criterion: 25). The IGA score as assessed by the investigator at baseline was 3.3-3.4, indicating moderate to severe acne at baseline. There were no statistically significant differences between the groups with regard to acne vulgaris severity. Similar baseline lesion counts were observed in the PP population and mPP population. (not shown).

A total of 150 subjects were randomized into 3 study groups (1%, 4% and Placebo), with 50 subjects in each group. Among all subjects, 139 subjects conformed to criteria for the ITT and safety (SAF) population: 46 in the 1% group, 47 in the 4% group and 46 in the Placebo group. Of these, 96 conformed to criteria for the PP population: 31 in 1% group, 36 in 4% group and 29 in the Placebo group.

TABLE 2.2
Disposition of subjects, all randomized subjects by study group

| | FXFM244 | | FXFM244 | | | | | |
|---|---|---|---|---|---|---|---|---|
| | 1% | | 4% | | Placebo | | Total | |
| | N = 50 | | N = 50 | | N = 50 | | N = 150 | |
| Subject Disposition | N | % | N | % | N | % | N | % |
| Subject screened | 50 | 100 | 50 | 100 | 50 | 100 | 150 | 100 |
| Subjects randomized | 50 | 100 | 50 | 100 | 50 | 100 | 150 | 100 |
| Subjects who completed | 31 | 62 | 36 | 72 | 29 | 58 | 96 | 64 |
| the study-per protocol | | | | | | | | |
| Subjects who did not | 19 | 38 | 14 | 28 | 21 | 42 | 54 | 36 |
| complete the study per | | | | | | | | |
| protocol | | | | | | | | |
| Reason for discontinuation | | | | | | | | |
| Randomized but did not | 4 | 8 | 3 | 6 | 4 | 8 | 11 | 7 |
| attend to post baseline visit | | | | | | | | |
| Consent withdrawal | 4 | 8 | 0 | 0 | 4 | 8 | 8 | 5 |
| Lost to follow up (LFU) | 4 | 8 | 5 | 10 | 4 | 8 | 13 | 9 |
| Other (protocol violation) | 1 | 2 | 1 | 2 | 1 | 2 | 3 | 2 |
| Other (non-compliance) | 1 | 2 | 0 | 0 | 1 | 2 | 2 | 1 |
| Other (out of visit 5 window) | 5 | 10 | 5 | 10 | 7 | 14 | 17 | 11 |

TABLE 2.3
Summary of analysis populations by study group

| | FXFM244 | | FXFM244 | | | |
|---|---|---|---|---|---|---|
| | 1% | | 4% | | Placebo | |
| | N = 50 | | N = 50 | | N = 50 | |
| Analysis Population | N | % | N | % | N | % |
| Safety Population(SAF)^{a} | 46 | 92 | 47 | 94 | 46 | 92 |
| ITT Population ^{b} | 46 | 92 | 47 | 94 | 46 | 92 |
| Per Protocol Population (PP) ^{c} | 31 | 62 | 36 | 72 | 29 | 58 |
| mPP Population ^{d} | 31 | 62 | 35 | 70 | 29 | 58 |

### 4. Statistical Methodology

All measured variables and derived parameters were tabulated by descriptive statistics. Descriptive statistics summary tables included sample size, absolute and relative frequency of categorical variables and sample size, arithmetic mean, standard deviation, median, minimum and maximum for means of continuous variables per group.

All statistical tests were analyzed to a significance level of 0.05.

### Demographic and Baseline Characteristics Display

The planned sample size of 150 subjects was selected based on prior literature acne studies, to provide adequate information on the effect of an anti-acne topical drug. No formal sample size calculation was performed for this study. The demographic and baseline evaluation were done for the ITT population. For the demographic and baseline categorical variables (gender, physical examinations of heart, lungs and abdomen and medical history variables) numbers and percentages were calculated. Distributions for the categorical variables were compared and analyzed by the Chi square test. For the demographic and baseline continuous variable (age, height, weight, vital signs and disease duration) ranges, medians, means and standard deviations were calculated. The results between continuous variable were analyzed by ANOVA (Analysis of Variance) Bonferroni multiple comparison test (subgroups≥2).

### Efficacy Analysis

The efficacy evaluations were done for the Intent to Treat (ITT), the Per Protocol (PP) and the modified (mPP) populations. The efficacy endpoints were: (a) the change in the number (primary endpoint) and percentage (secondary endpoint) of lesion counts (inflammatory, non-inflammatory and total) at post baseline visits compared to baseline and (b) Investigator's Global Assessment (co-primary endpoint).

For the lesion count variables ranges, medians, means and standard deviations were calculated. Test for normality was done by Shapiro-Wilk normality test. As the null hypothesis that the lesion counts variables are normally distributed is rejected, therefore the lesion counts results were compared and analyzed with non-parametric tests. The results between the lesion counts variables were compared and analyzed by the Kruskal-Wallis test (a non-parametric test for subgroups≥2).

Another co-primary endpoint is the evaluation of the Investigator Global Assessment (IGA) at each visit; IGA is based on a scale of 0 to 4, where 0=clear, 1=almost clear, 2=mild, 3=moderate, 4=severe. For the IGA variables ranges, medians, means and standard deviations were calculated. As the null hypothesis that the IGA assessment variables are normally distributed, is not rejected, therefore the IGA assessments results were compared and analyzed with parametric tests. The results between the IGA assessment variables were compared and analyzed by the ANOVA (Analysis of Variance) Bonferroni multiple comparison test (a parametric test for subgroups≥2).

IGA success was evaluated as (a) the number and percentage of subjects who met the success criterion no. 1 of “clear or almost clear” (grades less than 2) at each post baseline visit and (b) the number and percentage of subjects who met the success criteria no. 2 “improvement of at least 2 grades from the baseline” at each post baseline visit. The last two IGA success criteria are categorical variables. The distributions for categorical variables were compared and analyzed by the Chi square test (a parametric test), or by Fisher-Irwin exact test (a non-parametric test).

Two secondary endpoints related to acne improvement assessment after 12 weeks of treatment compared to baseline were evaluated: (a) the Investigator's Global Improvement assessment, which is based on based on a scale of 1 to 5, where 1=Worsening, 2=No change, 3=Slight improvement, 4=Moderate improvement, 5=Excellent improvement and (b) Patient's Global Improvement Assessment which is based on a scale of 1 to 4, where 1=Worse than prior to study 2=Same as prior to study, 3=Slightly better than prior to study, 4=Much better than prior to study.

The Investigator's Global Improvement assessment and Patient's Global Improvement Assessment were calculated and analyzed as categorical variables, numbers and percentages were calculated. The distributions for the physician's global improvement assessment and patient's global improvement assessment categorical variables were compared and analyzed by the Chi square test (a parametric test), or by Fisher-Irwin exact test (a non-parametric test for small numbers).

Subject compliance was calculated as the percentage of number of days the subject actually administrated the study drug to the sum of days of study drug administration plus days with no study drug administration.

Drug compliance was calculated as the mean of study drug weight used per day (the difference of containers' weight before and after use divided by the total number of days study drug was used).

### Safety and Tolerability Analysis

The safety and the tolerability analysis were done for the safety/ITT population. The safety analysis evaluated AEs, vital signs and physical examination.

Adverse events (AEs) were coded by the CTCAE (version 4.0; equivalent to MedDRA version 12), SOC (System Organ Class), preferred term and grade. Incidences of AEs were presented by serious AEs (SAEs), severity (grade), relationship to study drug, duration, action taken and outcome of event. Intensity or severity of each adverse experience was characterized as follows: Mild=adverse experience which is easily tolerated; Moderate=Adverse experience sufficiently discomforting to interfere with daily activity; Severe=Adverse experience which prevents normal daily activities.

For the summary by severity, subjects who have multiple occurrences of the same AE were classified according to the worst reported severity of the AE. For the summary by relationship to study drug, subjects who have multiple occurrences of the same AE were classified according to the strongest reported relationship to study medication. The AE variables are categorical variables. For the AE categorical variables numbers and percentages were calculated. Distributions for categorical variables were compared and analyzed by the Chi square test (a parametric test), or by Fisher-Irwin exact test (a non-parametric test).

The tolerability variables (erythema, dryness, pigmentation, peeling and itching) used a scale of 0 to 3, where 0=None, 1=Mild 2=Moderate, 3=Severe. Tolerability assessment was evaluated as the most severe irritation score from all 5 parts of the face.

The tolerability variables were calculated and compared as categorical variables and analyzed by the Chi square test (a parametric test), or by Fisher-Irwin exact test (a non-parametric test for small numbers).

### 5. Clinical Response to Treatment

The clinical response to treatment, (clinical success or clinical failure) was derived from an efficacy evaluation using the following methods and scales:

### A. Number of Inflammatory and Non-Inflammatory Acne Lesions on the Face

At each study visit, the whole face area was assessed for number of acne lesions, change from base line and percent change from baseline in the cumulative number of lesions of each type inflammatory (papules, pustules and nodulocystic lesions) and non-inflammatory (open (blackhead) and closed (whitehead) comedones) as well total lesion counts (inflammatory plus non-inflammatory lesions).

### B. Investigator Global Assessment—Current Severity Assessment of Acne Sign/Symptom

The severity of each of the following signs/symptoms was assessed and measured at baseline and at all follow up visits using scale described above (see section 4-efficacy analysis). In addition acne exacerbation was evaluated and any change in the severity of the disease was described by the investigator.

### C. Investigator's Global Improvement Assessment

This score was determined by the investigator and represents the patient's overall change from baseline in the severity of acne. The change in the patient's condition was scored at each post baseline visit compared to baseline using the scale described above (see section 4—efficacy analysis). The patient was photographed at baseline to facilitate comparisons at each post-baseline visit.

### D. Patient's Global Improvement Assessment

This score was obtained from the patient only at the end of treatment visit (visit 5 week 12). It results from the question “Considering your acne just before starting treatment and considering your condition today, indicate the change you have experienced according to the scale below”. The scale is provided in section 4 Statistical Analysis (efficacy assessment) above.

### E. Photography

Photographs were taken at each study visit, for documentation purpose, using a digital camera always under the same conditions: front, left side and right side of the face. This aided in estimating lesion area, severity and clinical response.

### 6. Safety and Tolerability

Safety and tolerability were determined for all randomized patients by the investigator at each visit. Safety was assessed using different parameters such as vital signs (blood pressure, heart rate, temperature), physical examination of body systems (heart, lungs, abdomen and, where appropriate, other body systems as indicated), pregnancy potential, adverse events and concomitant medications. Intensity or severity of each adverse experience was characterized using the scale described above (see Section 4—Safety and tolerability assessment).

Significant findings present prior to the start of treatment were included in the relevant medical history CRF. Significant findings made after the start of study drug (or therapy) which met the definition of an adverse events were recorded on the adverse event CRF. Height and weight measurements were recorded at visit 1 (screening/baseline). Any medication or treatment administered during the study as well as changes in established dosages of concomitant medication were documented in the CRFs.

Women of childbearing potential underwent urine pregnancy test at visit 1 (Screening/Baseline). If at subsequent visits, pregnancy was suspected, urine pregnancy test was to be performed.

Tolerability was determined by clinical assessment of skin irritation using different parameters such as, erythema, dryness, pigmentation, peeling at each study visit and at follow up. Based on patient subjective assessment, itching was recorded.

### 7. Satisfaction

At study visit 5 (EOT), a questionnaire was filled out by subjects and the subjects' parents regarding usability and treatment satisfaction. Different parameters were assessed after treatment such as greasiness, shininess, stickiness, moistness of the skin, general feeling, odor, use of pump and control of the amount, general satisfaction from foam and recommendation.

### 8. Study Results

### 8.1. Study Population

The study was conducted at three centers. A total of a hundred and fifty patients clinically diagnosed with at least moderate acne vulgaris were randomized into three groups, with fifty patients in each group. One group received the 1% minocycline foam, the second group received the 4% minocycline foam and the third group received the placebo.

The study was double-blinded and neither the investigators, nor the patients and their parents nor their legal guardian, nor the Applicants knew what strength of medication was dispensed or if the patient received placebo. A dye was introduced in the placebo formulation so that the placebo foam looked like the foam containing minocycline.

### 8.2. Efficacy on Inflammatory Lesions

The effect of the investigated products on inflammatory lesions is detailed in tables 8.2.1, 8.2.2 and 8.2.3 below. The study enrolled patients having moderate severe to severe acne: the mean number of inflammatory lesions was higher than that in many of the studies conducted on other acne-related drugs. The mean lesion counts at baseline was about 34.5 for inflammatory lesions whereas the eligibility criterion was 20. Clinical assessment of acne lesions at baseline indicated no significant differences in lesion counts between groups (mean total ˜80 lesions, including mean ˜37 inflammatory lesions in subjects receiving the 1% minocycline foam, and ˜34 in the subjects receiving the 4% minocycline foam and Placebo).

Nevertheless, a substantial decrease in the number of inflammatory lesions was observed in all groups, with better results observed in the groups that received a topical minocycline treatment.

TABLE 8.2.1
Mean number of inflammatory lesions over
16 weeks for each treatment (count by visit).

| Lesion Numbers | Weeks | | | | | |
|---|---|---|---|---|---|---|
| (mean) | 0 | 3 | 6 | 9 | 12 | 16 |
| Placebo | 33.6 | 21.1 | 17.8 | 16.8 | 17.2 | 16.0 |
| MCH 1% | 36.5 | 23.2 | 15.3 | 14.9 | 13.1 | 13.3 |
| MCH 4% | 33.5 | 16.6 | 10.4** | 9.3** | 9.8** | 9.7** |

**Statistically different from placebo (p < 0.005, ITT population).

TABLE 8.2.2
Mean change from baseline in the number of inflammatory
lesions over 16 weeks for each treatment.

| Mean Lesion Number | | | | | |
|---|---|---|---|---|---|
| Change from Baseline | | | Weeks | | |
| | 3 | 6 | 9 | 12 | 16 |
| Placebo | −12.5 | −15.8 | −16.8 | −16.5 | −17.6 |
| MCH 1% | −13.3 | −21.2** | −21.6** | −23.4** | −23.2** |
| MCH 4% | −16.9** | −23.2** | −24.2** | −23.7** | −23.9** |

**Statistically different from placebo (p < 0.05) for ITT population).

TABLE 8.2.3
Mean percentage of decrease from baseline in the number
of inflammatory lesions over 16 weeks for each treatment.

| Mean % Number Change from Baseline | | | Weeks | | |
|---|---|---|---|---|---|
| | 3 | 6 | 9 | 12 | 16 |
| Placebo | −40% | −50% | −52% | −51% | −54% |
| MCH 1% | −39% | −59% | −61% | −67%** | −65% |
| MCH 4% | −53%** | −71%** | −74%** | −72%** | −73%** |

**Statistically different from placebo (p < 0.05, ITT population).

TABLE 8.2.4
Portion of subjects with 50%, 60%, 70%, or 80% reduction
in inflammatory lesions by study group and visit (ITT Population)

| | | FXFM244 | | FXFM244 | | | | P value |
|---|---|---|---|---|---|---|---|---|
| | | % 1 | | % 4 | | Placebo | | (FXFM244 |
| | | N = 46 | | N = 47 | | N = 46 | | 4% vs. |
| | | N | % | N | % | N | % | Placebo) |
| More than 50% | 6 weeks | 35 | 76.1 | 37 | 78.7 | 24 | 52.2 | 0.0070 |
| reduction | 9 weeks | 33 | 71.7 | 39 | 83.0 | 26 | 56.5 | 0.0050 |
| | 12 weeks | 36 | 78.3 | 40 | 85.1 | 26 | 56.5 | 0.0020 |
| | 16 weeks | 34 | 73.9 | 39 | 83.0 | 28 | 60.9 | 0.0180 |
| More than 60% | 6 weeks | 26 | 56.5 | 34 | 72.3 | 19 | 41.3 | 0.0030 |
| reduction | 9 weeks | 26 | 56.5 | 39 | 83.0 | 21 | 45.7 | 0.0001 |
| | 12 weeks | 32 | 69.6 | 40 | 85.1 | 23 | 50.0 | 0.0001 |
| | 16 weeks | 31 | 67.4 | 37 | 78.7 | 26 | 56.5 | 0.0220 |
| More than 70% | 6 weeks | 21 | 45.7 | 30 | 63.8 | 14 | 30.4 | 0.0010 |
| reduction | 9 weeks | 19 | 41.3 | 32 | 68.1 | 16 | 34.8 | 0.0010 |
| | 12 weeks | 25 | 54.4 | 33 | 70.2 | 13 | 28.3 | 0.0001 |
| | 16 weeks | 24 | 52.2 | 35 | 74.5 | 16 | 34.8 | 0.0001 |
| More than 80% | 6 weeks | 10 | 21.7 | 20 | 42.6 | 10 | 21.7 | 0.0320 |
| reduction | 9 weeks | 10 | 21.7 | 21 | 44.7 | 8 | 17.4 | 0.0050 |
| | 12 weeks | 15 | 32.6 | 22 | 46.8 | 7 | 15.2 | 0.0010 |
| | 16 weeks | 16 | 34.8 | 28 | 59.6 | 13 | 28.3 | 0.0020 |

The above results show a dose dependent effect on topical minocycline on the treatment of acne.

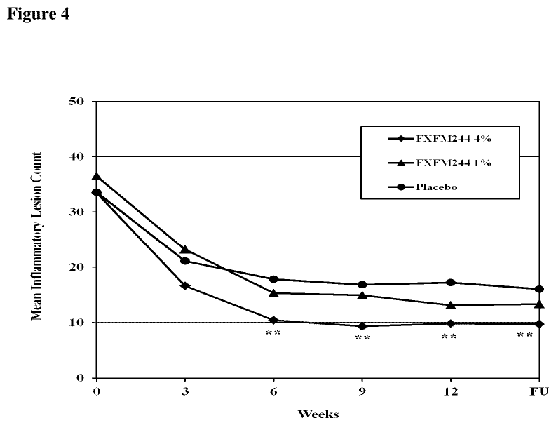

The mean number of inflammatory lesions decreased over time in all study groups. Starting from 6 weeks of treatment and onwards, a statistically significant difference (ITT population p=0.002) was observed in the number of inflammatory lesions between the 4% treatment group and Placebo (see Table 8.2.1 and FIG. 4). Similar results were observed in the PP and mPP populations (not shown).

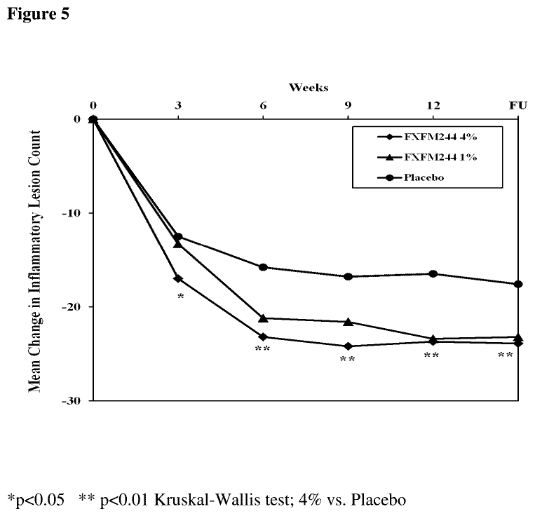

Analysis of absolute change in lesion count by visit revealed a clinically and statistically significant mean reduction (p=0.0286, ITT population) in the number of inflammatory lesions between subjects treated with the 4% minocycline foam compared to subjects treated with the Placebo already after 3 weeks of treatment i.e. a mean reduction of 16.9 inflammatory lesions vs. 12.5 lesions inflammatory, respectively. (see Table 8.2.2 and FIG. 5). In the PP and mPP populations the difference in change was significant from the 6 weeks time point (not shown).

As shown in FIG. 5 the mean change (decrease) in the number of inflammatory lesions in subjects receiving minocycline foam 4% started to flatten out after 6 weeks of treatment. This reduction was statistically significant between subjects receiving the 4% minocycline foam compared to subjects receiving the placebo—i.e a mean reduction of 23.2 inflammatory lesions vs. 15.8 inflammatory lesions for the Placebo (p<0.0003, ITT population). At the same time point a statistically significant reduction in the number of inflammatory lesions in subjects receiving 1% minocycine foam was also attained compared subjects receiving the Placebo—i.e. a mean reduction of 21.2 lesions vs. 15.8 lesions, respectively (p=0.0019, ITT population). These effects continued up to the follow-up visit. (see Table 8.2.2 and FIG. 5)

Statistical analysis at 12 weeks of treatment demonstrated a significantly higher reduction in inflammatory lesions in subjects receiving both 1% and 4% minocycline foam compared to subjects receiving Placebo; (i.e a mean reduction of ˜23 lesions in subjects receiving both 1% and 4% vs. a mean reduction of ˜16 lesions in subjects receiving placebo (p<0.005, ITT population).

Patients who received 4% topical minocycline had a higher percentage of decrease in the number of inflammatory lesions starting from week 3 throughout the study than those who were treated with 1% topical minocycline (Table 8.2.3).

Patients who received 4% topical minocycline showed a percentage of decrease in the number of inflammatory lesions of about 70% or more starting from week 6, which is better that the results obtained by oral treatments with minocycline (˜50% decrease), according to the information published on the prescription leaflets of commercially available oral minocycline treatments, which is provided below.

Patients who received 1% topical minocycline showed a percentage of decrease in the number of inflammatory lesions of about 60% or more starting from week 6, which is also better that the results obtained by oral treatments with minocycline (˜50% decrease), according to the information published on the prescription leaflets of commercially available oral minocycline treatments.

Statistical analysis of the results at week 3 revealed that the mean percent reduction in number of inflammatory lesions from baseline after 3 weeks of treatment was 53% in the 4% dose group, compared with 40% in the Placebo group was statistically significant (p=0.045, ITT population). After 6 weeks of treatment the mean percent reduction number of inflammatory lesions reached ˜71% in the 4% group, while the 1% and Placebo afforded 59% and 50% reduction respectively. The effect in the 4% group was statistically significantly higher than the Placebo (p=0.0007, ITT population); and it was also higher than 1% group (p=0.0071, ITT population)(see Table 8.2.3).

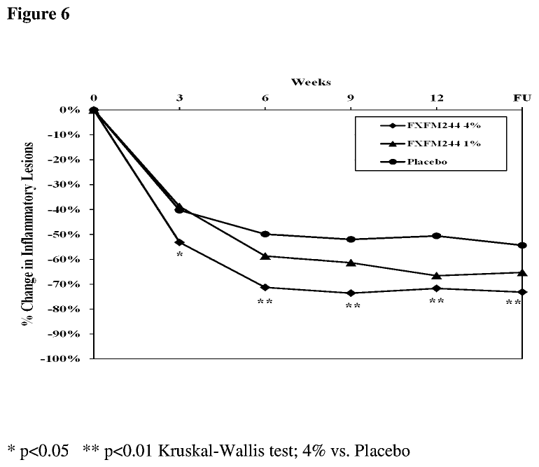

Statistical analysis of the results at week 12 further demonstrated that both the 1% topical minocycline and the 4% topical minocycline treatments gave statistically significant better results than placebo on the mean percentage of decrease from baseline in the number of inflammatory lesions. i.e the mean percent reduction in the number of inflammatory lesions of 72%, and 67% in subjects receiving both 4% and 1% respectively vs. the mean percent reduction of inflammatory lesions of 51% in subjects receiving placebo (p=0.0001, p=0.0072 respectively ITT population). Notably, the difference between the reduction in the 4% group and the Placebo was high—about 20%. This mean percent reduction continued 4 weeks after treatment in all treatment groups. (see Table 8.2.3 and FIG. 6).

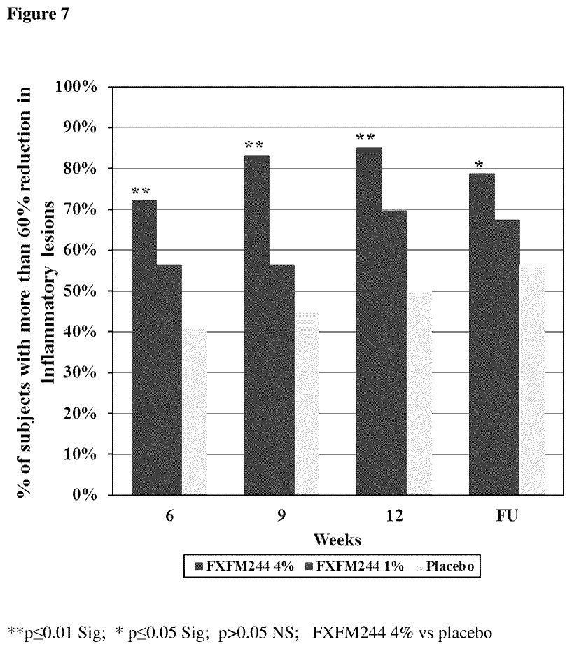

The percent of subjects who had a decrease of more than 50%, 60%, 70%, or 80% in the inflammatory lesions count was statistically significantly higher in the 4% treatment group compared to Placebo after 6 treatment weeks and onward (ITT population). For example, more than 50% reduction in the inflammatory lesion counts in more than 76% of the subject in each minocycline treatment group was reached following 6 weeks which was statistically significant reduction compared to Placebo 52% of the subjects. For example, after 9 weeks of treatment 83% of the subjects receiving the 4% minocycline foam had more than 60% reduction of inflammatory lesions, compared to 56.5% of the subjects receiving the 1% minocycline foam and 45.7% in the Placebo group (4% vs. Placebo: p=0.0001, ITT population) (see Table 8.2.4 and FIG. 7).

In sum, overall 4% was more effective than 1%, which in turn was more effective than placebo formulation, which in turn was more effective than no treatment.

### 8.3. Efficacy on Non-Inflammatory Lesions

The effect of the investigated products on non-inflammatory lesions is detailed in tables 8.3.1, 8.3.2, 8.3.3 and 8.3.4, below. As can be seen from the mean number non-inflammatory lesions at baseline, this study enrolled patients having acne of high severity: the mean number of lesions is higher that in many of the studies conducted on other acne-related drugs. The mean lesion counts at baseline was about 44.8 for non-inflammatory lesions whereas eligibility criterion was 25. Nevertheless, a substantial decrease in the number of non-inflammatory lesions was observed in all groups, with better results observed in the groups that received a topical minocycline treatment.

TABLE 8.3.1
Mean number of non-inflammatory lesions
over 16 weeks for each treatment.

| Lesion Numbers | Weeks | | | | | |
|---|---|---|---|---|---|---|
| (mean) | 0 | 3 | 6 | 9 | 12 | 16 |
| Placebo | 45.1 | 27.2 | 23.5 | 22.3 | 19.7 | 18.7 |
| MCH 1% | 46.2 | 32.4 | 24.7 | 22.0 | 18.7 | 16.7 |
| MCH 4% | 43.3 | 28.3 | 19.8 | 15.1 | 12.0** | 13.2 |

**Statistically different from placebo (p < 0.05, ITT population)

TABLE 8.3.2
Mean change from baseline in the number of non-
inflammatory lesions over 16 weeks for each treatment.

| Mean Lesion Number | | | | | |
|---|---|---|---|---|---|
| Change from Baseline | | | Weeks | | |
| | 3 | 6 | 9 | 12 | 16 |
| Placebo | −17.9 | −21.6 | −22.8 | −25.4 | −26.4 |
| MCH 1% | −13.8 | −21.5 | −24.2 | −27.4 | −29.5 |
| MCH 4% | −15 | −23.5 | −28.2 | −31.4 | −30.1 |

**Statistically different from placebo (p =< 0.05).

TABLE 8.3.3
Mean percentage of decrease from baseline in the number
of non-inflammatory lesions over 16 weeks for each treatment.

| Mean % number Change from Baseline | | | Weeks | | |
|---|---|---|---|---|---|
| | 3 | 6 | 9 | 12 | 16 |
| Placebo | −42% | −49% | −52% | −57% | −59% |
| MCH 1% | −34% | −51% | −56% | −65% | −68% |
| MCH 4% | −33% | −55% | −65% | −73%** | −69% |

**Statistically different from placebo (p < 0.05).

TABLE 8.3.4
Percent (%) of subjects with more than 50% reduction in
non-inflammatory lesion count from baseline by study group and visit

| % of Subjects with | | | | | | | |
|---|---|---|---|---|---|---|---|
| >50% Reduction in | FXFM244 | | FXFM244 | | | | |
| Non-Inflammatory | % 1 | | % 4 | | Placebo | | |
| Lesion Count | N = 46 | | N = 47 | | N = 46 | | |
| Visit no. | N | % | N | % | N | % | P value |
| Visit 3 (6 weeks) | 27 | 58.7 | 32 | 68.1 | 25 | 54.4 | a: 0.3830 |
| | | | | | | | b: 0.1740 |
| | | | | | | | c: 0.6740 |
| | | | | | | | d: 0.3470 |
| Visit 4 (9 weeks) | 29 | 63.0 | 35 | 74.5 | 26 | 56.5 | a: 0.1860 |
| | | | | | | | b: 0.0690 |
| | | | | | | | c: 0.5240 |
| | | | | | | | d: 0.2340 |
| Visit 5 (12 weeks) | 35 | 76.1 | 42 | 89.4 | 33 | 71.7 | a: 0.0870 |
| | | | | | | | b: 0.0380* |
| | | | | | | | c: 0.6350 |
| | | | | | | | d: 0.1060 |
| Visit 6 (FU) | 39 | 84.8 | 40 | 85.1 | 33 | 71.7 | a: 0.1800 |
| | | | | | | | b: 0.1170 |
| | | | | | | | c: 0.1290 |
| | | | | | | | d: 1.0000 |

*p ≤ 0.05 Sig; p > 0.05 NS; a: FXFM244 4% vs FXFM244 % 1 vs placebo
b: FXFM244 4% vs placebo; c: FXFM244 1% vs placebo; d: FXFM244 4% vs FXFM244 1%

TABLE 8.3.5
Percent (%) of subjects with more than 70% reduction in
non-inflammatory lesion count from baseline by study group and visit

| % of Subjects with | | | | | | | |
|---|---|---|---|---|---|---|---|
| >70% Reduction in | FXFM244 | | FXFM244 | | | | |
| Non-Inflammatory | % 1 | | % 4 | | Placebo | | |
| Lesion Count | N = 46 | | N = 47 | | N = 46 | | |
| Visit no. | N | % | N | % | N | % | P value |
| Visit 3 (6 weeks) | 16 | 34.8 | 18 | 38.3 | 18 | 39.1 | a: 0.9010 |
| | | | | | | | b: 0.9340 |
| | | | | | | | c: 0.6660 |
| | | | | | | | d: 0.7250 |
| Visit 4 (9 weeks) | 19 | 41.3 | 23 | 48.9 | 18 | 39.1 | a: 0.6040 |
| | | | | | | | b: 0.3410 |
| | | | | | | | c: 0.8320 |
| | | | | | | | d: 0.4600 |
| Visit 5 (12 weeks) | 27 | 58.7 | 27 | 57.5 | 17 | 37.0 | a: 0.0640 |
| | | | | | | | b: 0.0480* |
| | | | | | | | c: 0.0370* |
| | | | | | | | d: 0.9030 |
| Visit 6 (FU) | 29 | 63.0 | 29 | 61.7 | 21 | 45.7 | a: 0.1720 |
| | | | | | | | b: 0.1210 |
| | | | | | | | c: 0.0940 |
| | | | | | | | d: 0.8940 |

*p ≤ 0.05 Sig; p > 0.05 NS; a: FXFM244 4% vs FXFM244 % 1 vs placebo
b: FXFM244 4% vs placebo; c: FXFM244 1% vs placebo; d: FXFM244 4% vs FXFM244 1%

Statistical analysis of average non-inflammatory lesion counts and also of change in mean lesion number demonstrated a statistically significant change in subjects who received 4% minocycline foam compared to Placebo at 12 weeks (see Tables 8.3.1 and 8.3.2 respectfully).

The above results show a dose dependent effect on topical minocycline on the treatment of acne. The patients who received 4% topical minocycline had a higher percentage of decrease in the number of non-inflammatory lesions starting from week 3 throughout the study than those who were treated with 1% topical minocycline (see Table 8.3.3).

Patients who received 4% topical minocycline showed a percentage of decrease in the number of non-inflammatory lesions of about 65% or more starting from week 9 (see Table 8.3.3), which is better that the results obtained by oral treatments with minocycline (no effect), according to the information published on the prescription leaflets of commercially available oral minocycline treatments.

Patients who received 1% topical minocycline showed a percentage of decrease in the number of non-inflammatory lesions of about 50% or more starting from week 6, which is better that the results obtained by oral treatments with minocycline (no effect), according to the information published on the prescription leaflets of commercially available oral minocycline treatments.

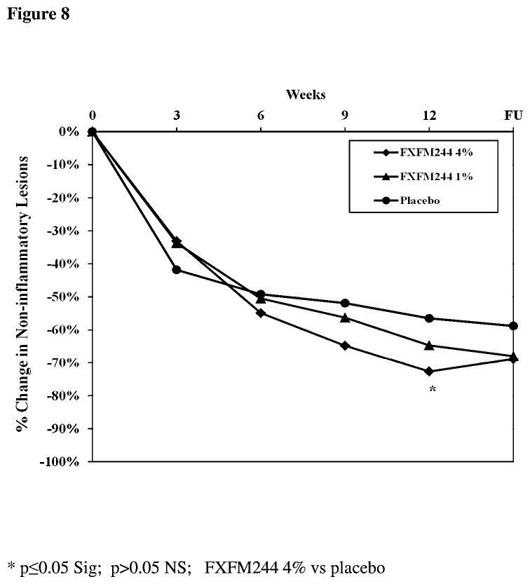

Statistical analysis of the results at week 12 further demonstrated that the 4% topical minocycline treatment gave significantly better results than placebo on the mean percentage of decrease from baseline in the number of non-inflammatory lesions; (i.e the mean percent reduction of 73% in the number of non-inflammatory lesions in subjects receiving 4% vs. the mean percent reduction of 57% in subjects receiving placebo (p<0.05, ITT population) (see Table 8.3.3 and FIG. 8)).

The percent of subjects who had a decrease of more than 50% and 70% in non-inflammatory lesion counts was significantly higher in subjects receiving the 4% minocycline foam compared to Placebo at 12 weeks visit in the ITT population. (see Table 8.3.4 and Table 8.3.5).

### 8.4. Efficacy on Total Lesions

The effect of the investigated products on total lesions (inflammatory and non-inflammatory) is detailed in tables 8.4.1-8.4.6 below. As can be seen from the mean number total lesions at baseline, this study enrolled patients having moderate to severe acne: the mean number of total lesions (80) is higher than that seen in many of the studies conducted on other acne-related drugs. Nevertheless, a substantial decrease in the number of total lesions was observed in all groups, with better results observed in the groups that received a topical minocycline treatment.

TABLE 8.4.1
Mean number of total lesions
over 16 weeks for each treatment.

| Lesion Numbers | Weeks | | | | | |
|---|---|---|---|---|---|---|
| (mean) | 0 | 3 | 6 | 9 | 12 | 16 |
| Placebo | 78.7 | 48.3 | 41.3 | 39.1 | 36.9 | 34.7 |
| MCH 1% | 82.7 | 55.6 | 40.0 | 37.0 | 31.8 | 30.0 |
| MCH 4% | 76.9 | 44.7 | 30.1** | 23.9** | 21.7** | 23.2** |

**Statistically different from placebo (p < 0.05).

TABLE 8.4.2
Mean Change from baseline in the mean number of total
lesions over 16 weeks for each treatment.

| Lesion Number Change from Baseline | | | Weeks | | |
|---|---|---|---|---|---|
| | 3 | 6 | 9 | 12 | 16 |
| Placebo | −30.4 | −37.4 | −39.6 | −41.9 | −44 |
| MCH 1% | −27.1 | −42.7 | −45.7 | −50.8 | −52.7 |
| MCH 4% | −32.2 | −46.8 | −52.4** | −55.1** | −53.7 |

**Statistically different from placebo (p < 0.05) ITT.

TABLE 8.4.3
Mean Percentage of decrease from baseline in the mean
number of total lesions over 16 weeks for each treatment.

| Mean % | | | | | |
|---|---|---|---|---|---|
| Number | | | | | |
| Change from | Weeks | | | | |
| Baseline | 3 | 6 | 9 | 12 | 16 |
| Placebo | −41% | −49% | −52% | −54% | −57% |
| MCH 1% | −36% | −54% | −58% | −64% | −66% |
| MCH 4% | −42% | −62%** | −69%** | −72%** | −71%** |

**Statistically different from placebo (p < 0.05).

TABLE 8.4.4
Percent (%) of subjects who had more than 50% reduction
in total lesion count from baseline by study group and visit (ITT/LOCF)

| % of Subjects with | FXFM244 | | FXFM244 | | | | |
|---|---|---|---|---|---|---|---|
| >50% Reduction in | % 1 | | % 4 | | Placebo | | |
| Total Lesion Count | N = 46 | | N = 47 | | N = 46 | | |
| Visit no. | N | % | N | % | N | % | P value |
| Visit 3 (6 weeks) | 31 | 67.4 | 32 | 68.1 | 28 | 60.9 | a: 0.7230 |
| | | | | | | | b: 0.4670 |
| | | | | | | | c: 0.5140 |
| | | | | | | | d: 0.9430 |
| Visit 4 (9 weeks) | 30 | 65.2 | 38 | 80.9 | 27 | 58.7 | a: 0.0610 |
| | | | | | | | b: 0.0200* |
| | | | | | | | c: 0.5190 |
| | | | | | | | d: 0.0890 |
| Visit 5 (12 weeks) | 32 | 69.6 | 42 | 89.4 | 30 | 65.2 | a: 0.0120* |
| | | | | | | | b: 0.0070** |
| | | | | | | | c: 0.6560 |
| | | | | | | | d: 0.0220* |
| Visit 6 (FU) | 36 | 78.3 | 38 | 80.9 | 30 | 65.2 | a: 0.1780 |
| | | | | | | | b: 0.0890 |
| | | | | | | | c: 0.1650 |
| | | | | | | | d: 0.7570 |

*p ≤ 0.05; Sig; p > 0.05 NS; a: FXFM244 4% vs FXFM244 % 1 vs placebo
b: FXFM244 4% vs placebo; c: FXFM244 1% vs placebo; d: FXFM244 4% vs FXFM244 1%

TABLE 8.4.5
Percent (%) of subjects with more than 60% reduction in
total lesion count from baseline by study group and visit

| % of Subjects with | FXFM244 | | FXFM244 | | | | |
|---|---|---|---|---|---|---|---|
| >60% Reduction in | % 1 | | % 4 | | Placebo | | |
| Total Lesion Count | N = 46 | | N = 47 | | N = 46 | | |
| Visit no. | N | % | N | % | N | % | P value |
| Visit 3 (6 weeks) | 21 | 45.7 | 28 | 59.6 | 20 | 43.5 | a: 0.2410 |
| | | | | | | | b: 0.1200 |
| | | | | | | | c: 0.8340 |
| | | | | | | | d: 0.1790 |
| Visit 4 (9 weeks) | 23 | 50.0 | 36 | 76.6 | 21 | 45.7 | a: 0.0050** |
| | | | | | | | b: 0.0020** |
| | | | | | | | c: 0.6760 |
| | | | | | | | d: 0.0080** |
| Visit 5 (12 weeks) | 31 | 67.4 | 38 | 80.9 | 26 | 56.5 | a: 0.0410* |
| | | | | | | | b: 0.0110* |
| | | | | | | | c: 0.2830 |
| | | | | | | | d: 0.1380 |
| Visit 6 (FU) | 33 | 71.7 | 34 | 72.3 | 27 | 58.7 | a: 0.2850 |
| | | | | | | | b: 0.1660 |
| | | | | | | | c: 0.1890 |
| | | | | | | | d: 0.9480 |

**p ≤ 0.01 Sig; *p ≤ 0.05 Sig; p > 0.05 NS; a: FXFM244 4% vs FXFM244 % 1 vs placebo
b: FXFM244 4% vs placebo; c: FXFM244 1% vs placebo; d: FXFM244 4% vs FXFM244 1%

TABLE 8.4.6
Percent (%) of subjects with more than 70% reduction in total
lesion count from baseline by study group and visit (ITT/LOCF)

| % of Subjects with | FXFM244 | | FXFM244 | | | | |
|---|---|---|---|---|---|---|---|
| >70% Reduction in | % 1 | | % 4 | | Placebo | | |
| Total Lesion Count | N = 46 | | N = 47 | | N = 46 | | |
| Visit no. | N | % | N | % | N | % | P value |
| Visit 3 (6 weeks) | 16 | 34.8 | 22 | 46.8 | 12 | 26.1 | a: 0.1120 |
| | | | | | | | b: 0.0380* |
| | | | | | | | c: 0.3650 |
| | | | | | | | d: 0.2380 |
| Visit 4 (9 weeks) | 19 | 41.3 | 26 | 55.3 | 18 | 39.1 | a: 0.2340 |
| | | | | | | | b: 0.1180 |
| | | | | | | | c: 0.8320 |
| | | | | | | | d: 0.1760 |
| Visit 5 (12 weeks) | 26 | 56.5 | 28 | 59.6 | 14 | 30.4 | a: 0.0090** |
| | | | | | | | b: 0.0050** |
| | | | | | | | c: 0.0120* |
| | | | | | | | d: 0.7650 |
| Visit 6 (FU) | 24 | 52.2 | 28 | 59.6 | 18 | 39.1 | a: 0.1370 |
| | | | | | | | b: 0.0490* |
| | | | | | | | c: 0.2090 |
| | | | | | | | d: 0.4720 |

*p ≤ 0.05 Sig; p > 0.05 NS; a: FXFM244 4% vs FXFM244 % 1 vs placebo
b: FXFM244 4% vs placebo; c: FXFM244 1% vs placebo; d: FXFM244 4% vs FXFM244 1%

Statistical analysis of the total lesion counts revealed that significantly fewer lesions were observed in subjects receiving 4% minocycline foam compared to Placebo from visit 6 weeks and onwards in the ITT population (Table 8.4.1). In PP and mPP population the difference between 4% treatment group and Placebo was significant at visit 9 and 12 weeks (not shown).

Statistical analysis of the results at week 12 demonstrated a statistically significant higher mean reduction in the number of total lesions in subjects receiving 4% minocycline foam compared to subjects receiving Placebo; i.e a mean reduction of ˜55 lesions vs. ˜42 lesions in the placebo group (p<0.01, ITT population). (see Table 8.4.2).

Statistical analysis of the results at week 9 demonstrated a statistically significant higher mean reduction in the number of total lesions in subjects receiving 4% minocycline foam compared to subjects receiving Placebo; i.e a mean reduction of ˜52 lesions vs. ˜40 lesions in the placebo group (p<0.05, ITT population). (see Table 8.4.2).

The above results show a dose dependent effect of topical minocycline on the treatment of acne. The patients who received 4% topical minocycline had a higher percentage of decrease in the number of total lesions starting from week 3 throughout the study than those who were treated with 1% topical minocycline (see Table 8.4.3).

Patients who received 4% topical minocycline showed a percentage of decrease in the number of total lesions of about 60% or more starting from week 6.

Patients who received 1% topical minocycline showed a percentage of decrease in the number of total lesions of about 50% or more starting from week 6. Statistical analysis of the results at week 12 further demonstrated that the 4% topical minocycline treatment gave statistically significant better results than placebo on the mean percentage of decrease from baseline in the number of total lesions . . . i.e the mean percent reduction of 72% in the total number of lesions in subjects receiving 4% vs. the mean percent reduction of 54% in subjects receiving placebo (p<0.01, ITT population)(see Table 8.4.3) and the difference between the groups was statistically significant

Similar results were observed in the PP and mPP population; however, in these populations there was also a statistically significant difference in the reduction of inflammatory lesions between the 4% and 1% minocycline treatment groups (not shown).

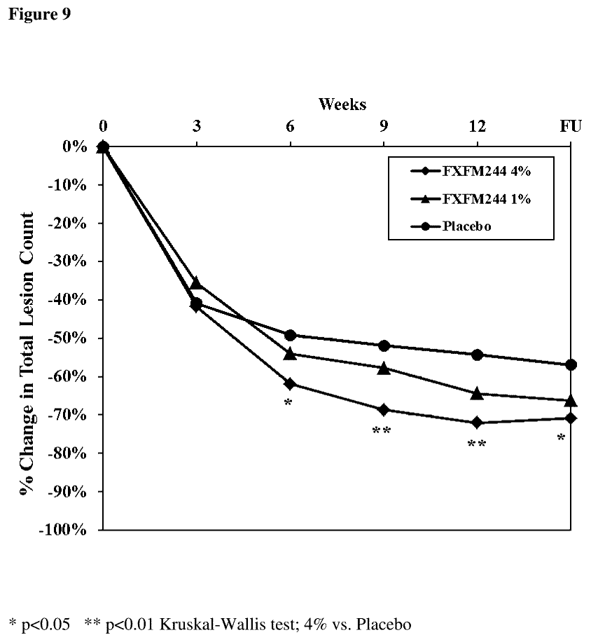

A statistically clinically and significant mean percent reduction in the the number of lesions observed in subjects receiving 4% dose group (−62%) compared to Placebo (49%) from visit 6 weeks and onwards in the ITT population (Table 8.4.3 and FIG. 9).

The percent of subjects who had a decrease of more than 50%, 60% and 70% in the total lesion counts was significantly higher in subjects receiving the 4% minocycline foam compared to Placebo at 12 weeks visit in the ITT and PP populations (Tables 8.4.4-8.4.6). After 9 weeks of treatment 76.6% of the subjects receiving the 4% minocycline foam had more than 60% reduction of total lesions, compared to 50% subjects receiving the 1% minocycline foam and 45.7% in the Placebo group (4% vs. Placebo: p=0.002, ITT population) (Table 8.4.5).

### 8.5. Efficacy According to Investigator's Global Assessment and Investigator's and Patient's Global Improvement Assessment:

The effect of the investigated products on total lesions is detailed in tables 8.5.1, 8.5.2, 8.5.3, 8.5.4, 8.5.5, 8.5.6 and 8.5.7 below. As can be seen from the mean Investigator's Global Assessment, this study enrolled patients having acne of high severity: 3.3-3.4 out of 4 grades.

TABLE 8.5.1
Mean IGA over 16 weeks for each treatment.

| | Weeks | | | | | |
|---|---|---|---|---|---|---|
| Mean IGA | 0 | 3 | 6 | 9 | 12 | 16 |
| Placebo | 3.4 | 2.7 | 2.5 | 2.5 | 2.4 | 2.3 |
| MCH 1% | 3.4 | 2.8 | 2.5 | 2.3 | 2.1 | 2.1 |
| MCH 4% | 3.3 | 2.6 | 2** | 1.9** | 1.7** | 1.8** |

**IGA score was statistically significantly lower than the placebo (p < 0.05, ITT population).

TABLE 8.5.2
Mean Change from baseline in the mean IGA
over 16 weeks for each treatment.

| Mean IGA | | | | | |
|---|---|---|---|---|---|
| Score | | | | | |
| Change | | | | | |
| from | Weeks | | | | |
| Baseline | 3 | 6 | 9 | 12 | 16 |
| Placebo | −0.6 | −0.9 | −0.9 | −1 | −1.1 |
| MCH 1% | −0.6 | −0.9 | −1.1 | −1.3 | −1.3 |
| MCH 4% | −0.7 | −1.3** | −1.4** | −1.7** | −1.5** |

TABLE 8.5.3
Mean Percentage of decrease from baseline in the
mean IGA over 16 weeks for each treatment.

| Mean % | | | | | |
|---|---|---|---|---|---|
| IGA | | | | | |
| Score | | | | | |
| Change | | | | | |
| from | Weeks | | | | |
| Baseline | 3 | 6 | 9 | 12 | 16 |
| Placebo | 21% | 26% | 26% | 29% | 32% |
| MCH 1% | 18% | 26% | 32% | 38% | 38% |
| MCH 4% | 17% | 33% | 33% | 50% | 50% |

TABLE 8.5.4
Percentage of patients having an “almost clear” or “clear”
IGA (less than IGA = 2) over 16 weeks for each treatment.
% of patients with IGA “almost clear” or “clear” (less than IGA = 2)

| | 3 | 6 | 9 | 12 | 16 |
|---|---|---|---|---|---|
| Placebo | 4% | 21% | 26% | 20% | 33% |
| MCH 1% | 2% | 15% | 22% | 37% | 41% |
| MCH 4% | 9% | 38% | 49%*** | 53%*** | 55%*** |

***Statistically different from placebo
(p < 0.05, ITT, PP).

TABLE 8.5.5
Percentage of patients having an IGA change of at least 2
units over 16 weeks for each treatment.
% of patients with IGA change of at least 2 units

| | 3 | 6 | 9 | 12 | 16 |
|---|---|---|---|---|---|
| Placebo | 7% | 9% | 17% | 15% | 22% |
| MCH 1% | 4% | 7% | 11% | 22% | 22% |
| MCH 4% | 9% | 23% | 30% | 36%** | 34% |

**Statistically different from placebo (p < 0.021, ITT). Statistical Analysis was not performed at other time-points.

TABLE 8.5.6
Investigator's global improvement assessment after 12
weeks of treatment by study group (ITT Population)

| | FXFM244 | | FXFM244 | | | | |
|---|---|---|---|---|---|---|---|
| Investigator's | % 1 | | % 4 | | Placebo | | |
| assessment | N = 46 | | N = 47 | | N = 46 | | |
| 12 weeks | n | % | n | % | n | % | P value |
| Excellent | 24 | 52.2 | 30 | 63.8 | 15 | 32.6 | a: 0.0020** |
| Moderate | 17 | 37.0 | 12 | 25.5 | 13 | 28.3 | b: 0.0030** |
| Slight | 2 | 4.3 | 3 | 6.4 | 13 | 28.3 | c: 0.0040** |
| improvement | 3 | 6.5 | 2 | 4.3 | 2 | 4.3 | d: 0.6230 |
| No change | 0 | | 0 | 0 | 3 | 6.5 | |
| Worsening | 0 | | | | | | |

a: FXFM244 4% vs. FXFM244 1% vs. Placebo; b: FXFM244 4% vs. Placebo; c: FXFM244 1% vs. Placebo; d: FXFM244 4% vs. FXFM244 1%

TABLE 8.5.7
Patient's global improvement assessment after 12 weeks of
treatment by study group. (PP Population)

| Patient global | FXFM244 | | FXFM244 | | | | |
|---|---|---|---|---|---|---|---|
| assessment | 1% | | 4% | | Placebo | | |
| 12 weeks | N = 31 | | N = 36 | | N = 29 | | |
| n | n | % | n | % | n | % | P value |
| Much better | 12 | 38.7 | 20 | 55.5 | 8 | 27.6 | a: 0.2030 |
| Slightly better | 13 | 42.0 | 14 | 38.9 | 14 | 48.3 | b: 0.0460* |
| Same | 4 | 12.9 | 1 | 2.8 | 5 | 17.2 | c: 0.7930 |
| Worse | 1 | 3.2 | 1 | 2.8 | 2 | 6.9 | d: 0.2990 |
| No data | 1 | 3.2 | 0 | 0 | 0 | 0 | |

a: FXFM244 4% vs. FXFM244 1% vs. Placebo;
b: FXFM244 4% vs. Placebo;
c: FXFM244 1% vs. Placebo;
d: FXFM244 4% vs. FXFM244 1%.

Statistical analysis of the results after 12 weeks of treatment demonstrated that the mean IGA score was significantly lower (improved) in subjects who received the 4% topical minocycline compared to patients who received 1% topical minocycline and subjects who received Placebo: mean score 1.7 vs. 2.1 and 2.4, respectively (p<0.001, ITT population). The difference between the 4% group and the 1% group was also statistically significant (p<0.05, ITT population). (see Table 8.5.1)

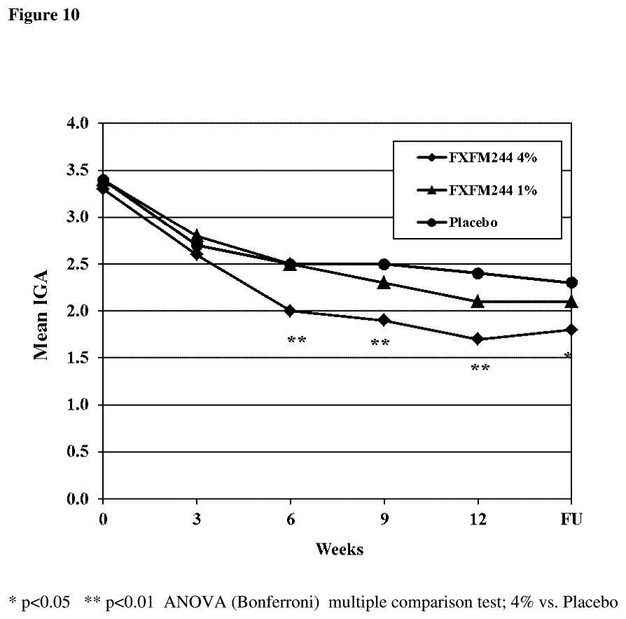

Mean IGA score was significantly lower (improved) in subjects receiving 4% minocycline foam compared to Placebo from 6 treatment weeks and onwards, including the follow-up visit, in the ITT population (mean score at 6 weeks: 2.0 vs. 2.5, respectively, p=0.01, Table 8.5.1, FIG. 10). In the PP population the beneficial effect of 4% minocycline foam was observed from 9 treatment weeks and onwards (not shown). Similar results were observed in analysis of mean change in IGA from baseline by visit, in the ITT population (mean IGA score change from baseline at 6 weeks: −1.3 vs. −0.9, respectively, p=0.005, Table 8.5.2).

The above results show a dose dependent effect of topical minocycline on the treatment of acne. The patients who received 4% topical minocycline had a higher percentage of decrease in the IGA starting from week 6 throughout the study than those who were treated with 1% topical minocycline (see Table 8.5.3).

Patients who received 4% topical minocycline showed a percentage of patients with IGA “almost clear” or “clear” of about 50% or more starting from before week 12, which is better that the results obtained by oral treatments with minocycline (˜17% at week 12), according to the information published on the prescription leaflets of commercially available oral minocycline treatments (see Table 8.5.4).

Patients who received 1% topical minocycline showed a percentage of patients with IGA “almost clear” or “clear” of about 37% or more starting from week 12, which is better that the results obtained by oral treatments with minocycline (˜17% at week 12), according to the information published on the prescription leaflets of commercially available oral minocycline treatments (see Table 8.5.4).

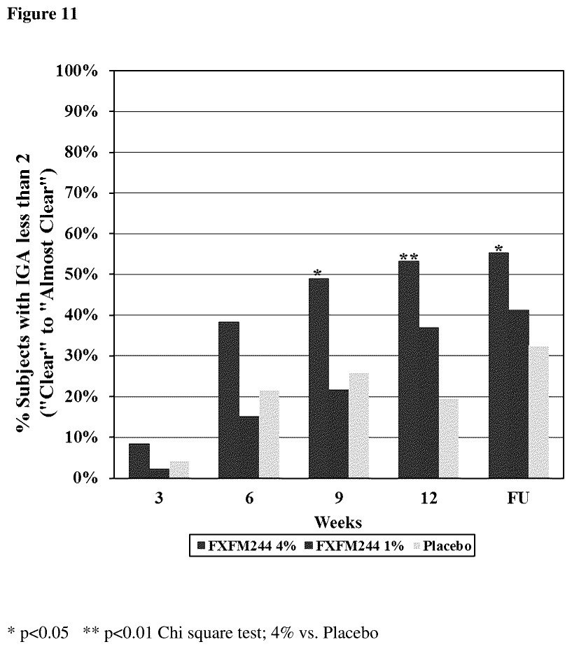

Statistical analysis of the results at week 12 further demonstrated the 4% topical minocycline treatments gave better results than placebo on the percentage of patients with IGA “almost clear” or “clear” (less than IGA=2) i.e. 53% of the subjects receiving 4% minocycline foam in the ITT population (see Table 8.5.4 and FIG. 11) and 61.1% in the PP population (not shown) demonstrated a score of less than 2 versus 20% of subjects receiving Placebo (ITT and PP populations)

Further, the percent of subjects with IGA scores less than 2 was significantly higher statistically in the patients receiving the 4% minocycline foam compared to pateints receiving the Placebo already from 9 treatment weeks onwards in the ITT population (49% vs. 26%, respectively, p=0.023, ITT population).

The corresponding percent of subjects with IGA scores of less than 2 in the 1% dose group at twelve weeks was higher than Placebo, but the difference was not statistically significant (37% vs. 20%, respectively, p=0.064, ITT population.). (see 8.5.4) Similar results were observed for the mPP population (not shown).

Further, the 4% topical minocycline treatment gave statistically significant better results than placebo on the percentage of patients with IGA change of at least 2 units i.e: 36% of the subjects receiving 4% minocycline foam vs. 15%, of the subjects receiving placebo respectively (p<0.021, ITT population) (see Table 8.5.5).

There was also a difference between the 1% topical minocycline treatment group and Placebo relating to IGA change of at least 2 units in all study populations but is was not statistically significant. Similar results were observed for the mPP population (not shown).

Investigator's Global Improvement Assessment after Twelve Weeks of Treatment

Approximately two thirds of subjects who received 4% minocycline foam and half of the subjects who received the 1% minocycline foam had ‘excellent’ improvement, compared to third of subjects in the Placebo group after twelve weeks of treatment (p<0.01, ITT population). Approximately 90% of subjects in both treatment groups (ITT population) had ‘excellent’ or ‘moderate’ improvement as assessed by the physician compared to 61% in the Placebo group. (See Table 8.5.6).

In the PP population, almost all subjects in the 4% dose group (97.2%) had ‘excellent’ or ‘moderate’ improvement (compared to 90.3% and 62.1% in the 1% and Placebo groups, respectively. (not shown)

Patient's Global Improvement Assessment after Twelve Weeks of Treatment

More than half of subjects who received the 4% minocycline foam evaluated their acne as “much better than prior to study”, compared to 28% who received the Placebo which is a statistically significant difference (p<0.05, PP population) (See Table 8.5.7). No statistically significant difference was observed between the 1% dose group and Placebo.

### Efficacy Conclusion

The baseline severity of acne in this study was moderate to severe, as judged by the number of acne lesions and investigator's global severity assessment (IGA). The mean number of inflammatory acne lesions at baseline was 35, and the mean number of non-inflammatory lesions was 45. These mean numbers were much higher than the minimum eligibility criteria of 20 inflammatory and 25 non-inflammatory lesions. The IGA score as assessed by the investigator at baseline was 3.3-3.4, indicating moderate to severe acne at baseline.

Daily application of topical minocycline foam (4% and 1%) on facial skin with moderate to severe acne resulted in a significant improvement of the disease as indicated by the primary and secondary endpoints of the study. There was a clinically and statistically significant reduction in the number of inflammatory, non-inflammatory and sum (total) acne lesions; and clinically and statistically significant improvement in the investigator global assessment of acne severity after 12 treatment weeks in the subjects receiving minocycline foam compared to Placebo. The effect of the drug was dose dependent, the effect 4% minocycline foam was generally greater than 1% minocycline foam.

Moreover, clinically and statistically significant reduction in the number of inflammatory lesions compared to baseline was already recorded after 3 treatment weeks in subjects receiving the 4% minocycline foam compared to Placebo.

Notably, the full effect of more than 70% reduction in inflammatory lesion counts was reached following 6 weeks of treatment subjects receiving the 4% minocycline foam compared to Placebo.

According to the investigator's global assessment at 12 weeks, more than half of the subjects had ‘clear’ or ‘almost clear’ skin in subjects receiving the 4% minocycline foam, compared to 37% in subjects receiving the 1% minocycline foam and only 20% of subjects in the Placebo group (p=0.001). Improvement of at least 2 grades in the investigator's global assessment score was also more frequent in subjects receiving the 4% minocycline foam 36% of the subjects compared to 22% in subjects receiving the 1% minocycline foam and only 15% of subjects in the Placebo group (p=0.02).

Secondary endpoint relating to acne improvement assessment by the investigator after 12 treatment weeks indicated ‘excellent’ improvement in approximately two thirds of subjects in subjects receiving the 4% minocycline foam and about half of the subjects receiving the 1% minocycline foam had, compared to about a third of subjects in the Placebo group (p=0.002). In addition, 55% of subjects receiving the 4% minocycline foam evaluated their acne as ‘much better than prior to study’ compared to 28% in the Placebo group (p=0.046, PP population).

The effect was most notably shown on inflammatory acne lesions in subjects receiving the 4% minocycline foam.

### 8.6. Safety and Tolerability

Generally, the placebo, the Minocycline Foam 1% and Minocycline Foam 4% were very well tolerated. Very few skin irritation events were recorded, and they were all transient. The severity of these events was primarily mild. No drug related systemic adverse events were noted throughout the study.

### Safety

During the course of the study only five adverse events were observed of which only two were skin related. The five adverse events were in the ITT population (3.6%), one in the 1% minocycline group, and two in each of the other groups. All adverse events were considered transient and mild in severity. None were considered to have been related to study treatment and none were considered serious adverse events. There were no notable differences between study groups.

### Tolerability

Tolerability of study treatment was evaluated using the following parameters:erythema, dryness, pigmentation, peeling, and itching using the scale in section 4 above. The score recorded was the most severe irritation score from all 5 parts of the face evaluated.

Overall, no statistically significant differences were demonstrated between treatment groups in any of the selected skin tolerance parameters during the course of the present study (p≥0.1970) (see Table 8.6.1 below). Very few subjects were found to have erythema at baseline, all mild and one moderate. There were sporadic erythema cases post-baseline, with no differences between treatment groups, nearly all mild and transient. After twelve weeks of treatment one mild case was found in each group of subjects receiving minocycline foam, however at F/U visit no subjects were found to have erythema.

Very few subjects were found to have skin dryness none at baseline through visit 3. Two cases of mild transient dryness were identified in the 1% group at visit 4, not seen at visit 5 onwards. After twelve weeks of treatment (visit 5) no skin dryness was observed. At the final evaluation, one subject in each of the 4% and Placebo groups had mild dryness.

Sporadic cases of pigmentation were observed over the course of the study, mostly occurring at visit 2 at similar frequencies in all three treatment groups. Nearly all cases were mild, except for one transient severe case at visit 2 in a subject receiving 1% minocycline foam. The investigator described these pigmentation cases as localized post inflammatory pigmentation, typical to the natural healing process of acne lesions. No cases were observed from visit 5 onwards. No notable differences were observed between treatment groups (not shown).

A total of five cases skin peeling were observed at visits 3, 4, and 6, all were mild and transient cases. At the final evaluation at visit 6, there were slightly more cases (2 cases) in the 4% group compared to with other treatments, but this was not statistically significant (p=0.7760).

No cases of itching related to the study were demonstrated through the study. One case of itching linked to herpes simplex was classified as an adverse event and was determined unrelated to the study.

The use of the minocycline foam was associated with a favorable safety and tolerability profiles in the concentrations and total exposures tested.

TABLE 8.6.1
Summary of skin tolerability parameters following
treatment with the tested articles

| | | |
|---|---|---|
| FXFM244 | FXFM244 | ^{a} P |

| | | | | |
|---|---|---|---|---|
| Tolerability | 1% | 4% | Placebo | value |

Erythema

| | | | | | |
|---|---|---|---|---|---|
| Baseline | n | 46 | 47 | 46 | 0.6220 |
| | No. of none | 44 | 46 | 46 | |
| | No. of mild | 1 | 1 | 0 | |
| | No. of | 1 | 0 | 0 | |
| | moderate | | | | |
| Visit 2 | n | 46 | 47 | 46 | 0.1970 |
| | No. of none | 45 | 43 | 44 | |
| | No. of mild | 0 | 4 | 1 | |
| | No. of | 1 | 0 | 1 | |
| | moderate | | | | |
| Visit 3 | n | 42 | 44 | 41 | 0.5240 |
| | No. of none | 39 | 43 | 39 | |
| | No. of mild | 3 | 1 | 2 | |
| Visit 4 | n | 37 | 41 | 35 | 1.0000 |
| | No.o f none | 36 | 40 | 35 | |
| | No. of mild | 1 | 1 | 0 | |
| Visit 5 | n | 36 | 41 | 36 | 1.0000 |
| | No. of none | 35 | 40 | 36 | |
| | No. of mild | 1 | 1 | 0 | |
| Visit 6 | n | 28 | 38 | 31 | 1.0000 |
| | No. of none | 28 | 38 | 31 | |

Dryness

| | | | | | |
|---|---|---|---|---|---|
| Baseline | n | 46 | 47 | 46 | 1.0000 |
| | No. of none | 46 | 47 | 46 | |
| Visit 2 | n | 46 | 47 | 46 | 1.0000 |
| | No. of none | 46 | 47 | 46 | |
| Visit 3 | n | 42 | 44 | 41 | 1.0000 |
| | No. of none | 42 | 44 | 41 | |
| Visit 4 | n | 37 | 41 | 35 | 0.1990 |
| | No. of none | 35 | 41 | 35 | |
| | No. of mild | 2 | 0 | 0 | |
| Visit 5 | n | 36 | 41 | 36 | 1.0000 |
| | No. of none | 36 | 41 | 36 | |
| Visit 6 | n | 28 | 38 | 31 | 1.0000 |
| | No. of none | 28 | 37 | 30 | |
| | No. of mild | 0 | 1 | 1 | |

Pigmentation

| | | | | | |
|---|---|---|---|---|---|
| Baseline | n | 46 | 47 | 46 | 0.6220 |
| | No.of none | 46 | 47 | 45 | |
| | No.of mild | 0 | 0 | 1 | |
| Visit 2 | n | 46 | 47 | 46 | 0.7890 |
| | No.of none | 42 | 45 | 43 | |
| | No.of mild | 3 | 2 | 3 | |
| | No.of severe | 1 | 0 | 0 | |
| Visit 3 | n | 42 | 44 | 41 | 1.0000 |
| | No.of none | 41 | 43 | 40 | |
| | No.of mild | 1 | 1 | 1 | |
| Visit 4 | n | 37 | 41 | 35 | 0.6370 |
| | No.of none | 36 | 41 | 35 | |
| | No.of mild | 1 | 0 | 0 | |
| Visit 5 | n | 36 | 41 | 36 | 1.0000 |
| | No.of none | 36 | 41 | 36 | |
| Visit 6 | n | 28 | 38 | 31 | 1.0000 |
| | No.of none | 28 | 38 | 31 | |

Peeling

| | | | | | |
|---|---|---|---|---|---|
| Baseline | n | 46 | 47 | 46 | 1.0000 |
| | No.of none | 46 | 47 | 46 | |
| Visit 2 | n | 46 | 47 | 46 | 1.0000 |
| | No.of none | 46 | 47 | 46 | |
| Visit 3 | n | 42 | 44 | 41 | 0.6540 |
| | No.of none | 41 | 44 | 41 | |
| | No.of mild | 1 | 0 | 0 | |
| Visit 4 | n | 37 | 41 | 35 | 0.6370 |
| | No.of none | 36 | 41 | 35 | |
| | No.of mild | 1 | 0 | 0 | |
| Visit 5 | n | 36 | 41 | 36 | 1.0000 |
| | No.of none | 36 | 41 | 36 | |
| Visit 6 | n | 28 | 38 | 31 | 0.7760 |
| | No.of none | 28 | 36 | 30 | |
| | No.of mild | 0 | 2 | 1 | |

Itching

| | | | | | |
|---|---|---|---|---|---|
| Baseline | n | 46 | 47 | 46 | 1.0000 |
| | No.of none | 46 | 47 | 46 | |
| Visit 2 | n | 46 | 47 | 46 | 1.0000 |
| | No.of none | 46 | 47 | 46 | |
| Visit 3 | n | 42 | 44 | 41 | 1.0000 |
| | No.of none | 42 | 44 | 41 | |
| Visit 4 | n | 37 | 41 | 35 | 1.0000 |
| | No.of none | 37 | 41 | 35 | |
| Visit 5 | n | 36 | 41 | 36 | 1.0000 |
| | No.of none | 36 | 41 | 36 | |
| Visit 6 | n | 28 | 38 | 31 | 1.0000 |
| | No.of none | 28 | 38 | 31 | |

p > 0.05 NS; ^{a} FXFM244 4% vs. FXFM244 % 1 vs. Placebo

### 8.7. Patients' Satisfaction

The degree of satisfaction of the patients from the treatment with the investigated products was evaluated based on patient's questionnaires results. The following scale was used to score patient's satisfaction: 0=Equal/indifferent; 1=Moderate; 2=High; 3=Very High; 4=extremely high.

In mean, patients indicated that they were moderately satisfied from the placebo treatment, whereas they were highly satisfied from both the 1% and 4% topical minocycline treatments.

In the Minocycline-4% group, 61% of patients stated that the efficacy was very high or high, and 27% rated the efficacy as moderate (median efficacy rating in this group was “high,” and was “moderate” in the placebo group). Furthermore, 68% of the patients rated the drug as very highly or highly better than other drugs they had formerly used.

As for ease of use of the drug, 76% of the patients said they were very-highly or highly satisfied. Patients noted that there was an oily feeling after use; however, 79% rated the overall after-use feeling as very-highly, highly, or moderately satisfactory. In comparison with former topical acne drugs, most of the patients (74%) rated Minocycline Foam as “better” than such drugs.

8.8. Literature Comparison with Current Anti-Acne Drugs.

The following tables 8.8.1 and 8.8.2 summarize the results obtained by an oral minocycline commercial product, by three additional recently-approved anti acne topical products and by the minocycline investigated products.

TABLE 8.8.1
Results obtained by anti-acne commercial products at Week 12.

| | Solodyn | Epiduo | Acanya | Ziana | Fabior |
|---|---|---|---|---|---|
| | (oral | (Adapalene + | (Clindamycin + | (Retinoic acid + | (Tazarotene |
| | Minocycline) | BPO) | BPO) | Clindamycin) | Foam) |
| % Change in | −44% | −47% | −55% | −54% | 57% |
| Inflammatory | | | | | |
| lesions | | | | | |
| % Change in | No effect | −49% | −43% | −43% | 55% |
| Non- | | | | | |
| inflammatory | | | | | |
| lesions | | | | | |
| % Patients | 17% | 30% | 29% | 31% | 29% |
| with IGA | | | | | |
| “Clear” or | | | | | |
| “Almost | | | | | |
| Clear” | | | | | |

TABLE 8.8.2
Results obtained by MCH 1% and MCH 4% at Week 12.

| | MCH 1% | MCH 4% |
|---|---|---|
| % Change in Inflammatory lesions | −67% | −72% |
| % Change in Non-inflammatory lesions | −65% | −73% |
| % Patients with IGA “Clear” or “Almost Clear” | 37% | 53% |

Both the 1% topical minocycline and 4% topical minocycline outperformed the results obtained by a commercial oral minocycline treatment. The results on the percentage of change in inflammatory lesions, non-inflammatory lesions, and on the percentage of patients with an with IGA of “Clear” or “Almost Clear” were better for the investigated topical minocycline in comparison with oral minocycline.

Further, both the 1% topical minocycline and 4% topical minocycline gave better results than Epiduo, Acanya, Ziana and Fabior in the percentage of change in inflammatory lesions, in the percentage of change in non-inflammatory lesions, and in the percentage of patients with an with IGA of “Clear” or “Almost Clear”.

### 9. Discussion

Although minocycline is one of the first line treatments for moderate to severe acne, it is currently available only in oral dosage forms. Oral minocycline, however, is associated with multiple unwanted side effects. The study medication, minocycline foam, is a stable minocycline hydrochloride topical product in a foam carrier. Applicants have shown that it can be used for the treatment of acne, rosacea, impetigo and other skin infections. The pressurized foam product facilitates easy application and even distribution of the drug, thereby improving treatment application, convenience and compliance.

This study shows the efficacy, tolerability and safety of topical minocycline in treating acne vulgaris on all the tested clinical endpoints. The baseline severity of acne in this study was moderate to severe, as judged by the number of acne lesions and investigator's global severity assessment (IGA). Daily application of the minocycline foam on facial acne skin areas resulted in a dose-dependent clinically and statistically significant improvement of the disease as indicated by the primary and secondary endpoints of the study.

A clinically and statistically significant reduction in the number and percentage of inflammatory, non-inflammatory and total lesions was observed, as well as clinically and statistically significant improvement in the IGA score after 12 treatment weeks compared to Placebo.

A statistically significant mean percent reduction in the number of inflammatory lesions after 12 weeks was demonstrated in subjects treated with minocycline foam (4% and 1%) compared to Placebo (−72%, and −67% compared to −51%, respectively). A statistically significant mean percent reduction in the number of non-inflammatory lesions count after 12 weeks was also demonstrated in subjects receiving the 4% minocyline foam compared to Placebo (−73%, vs. −57%, respectively). A statistically significant mean percent reduction in the total lesions count after 12 weeks was also demonstrated in subjects receiving the 4% minocyline foam compared to Placebo (−72%, vs. −54%, respectively).

The effect of the drug was dose dependent, the effect 4% minocycline foam was generally greater than 1% minocycline foam. A clinically and statistically significant reduction in the mean percent of inflammatory lesions (−53%) compared to baseline was already recorded after 3 treatment weeks in subjects receiving the 4% minocycline foam compared to Placebo (−40%). A clinically and statistically significant reduction in the mean percent of total lesions (−62%) compared to baseline was already recorded after six treatment weeks in subjects receiving the 4% minocycline foam compared to Placebo (49%).

Notably, more than a 70% reduction in inflammatory lesion counts was reached following 6 weeks of treatment subjects receiving the 4% minocycline foam which was a statistically significant reduction compared to Placebo.

The percent of subjects who had a decrease of more than 50%, 60%, 70%, or 80% in the inflammatory lesions count was statistically significantly higher in the 4% treatment group compared to Placebo after 6 treatment weeks and onward (ITT population). The percent of subjects who had a decrease of more than 50%, 60% or 70%, in the total lesions count was statistically significantly higher in the 4% treatment group compared to Placebo at twelve treatment weeks (ITT population). The percent of subjects who had a decrease of more than 50% or 70%, in the non-inflammatory lesions count was statistically significantly higher in the 4% treatment group compared to Placebo only at twelve treatment weeks (ITT population).

According to the investigator's global assessment at 12 weeks, more than half of the subjects had ‘clear’ or ‘almost clear’ skin in subjects receiving the 4% minocycline foam, compared to 37% in subjects receiving the 1% minocycline foam and only 20% of subjects in the Placebo group. The difference between subjects receiving 4% minocycline foam was considered statistically significant in comparison to placebo.

Improvement of at least 2 grades in the investigator's global assessment score at 12 weeks was also more frequent in subjects receiving the 4% minocycline foam 36% of the subjects compared to 22% in subjects receiving the 1% minocycline foam and only 15% of subjects in the Placebo group which was considered statistically significant. The difference between subjects receiving 4% minocycline foam was considered statistically significant in comparison to placebo.

Approximately two thirds of subjects who received 4% minocycline foam and half of the subjects who received the 1% minocycline foam had ‘excellent’ improvement, compared to third of subjects in the Placebo group after twelve weeks of treatment. The difference between subjects receiving minocycline foam was considered statistically significant in comparison to placebo.

In addition, more than half of subjects who received the 4% minocycline foam evaluated their acne as “much better than prior to study”, compared to 28% who received the Placebo which is a statistically significant difference.

Overall, the effect was most notably shown on inflammatory acne lesions in subjects receiving the 4% minocycline foam.

Use of the minocycline foam was associated with favorable safety and tolerability profiles in the concentrations and total exposures tested.

The tolerability was also excellent. Very few cases of erythema, dryness, and peeling were observed but they were mild and transient, and there was no difference in their occurrence among the study groups. No itching or pigmentation related to the drug was observed throughout the study.

The present topical minocycline compositions offer inter alia three significant advantages over oral minocycline:

1. Efficacy. Based on skin penetration studies conducted by applicants, as described in WO/2011/039637 the topical drug is available in the skin and may be available in higher amounts than the respective amounts of drug in the skin following oral intake. Furthermore an ex-vivo study described herein indicates minocycline specific targeting to the area of the hair follicle and sebaceous unit. This is consistent with the above results showing that topical MCH is more effective than oral minocycline in the treatment of acne, and more effective than the other anti-acne treatments detailed above. 2. Safety. Oral Minocycline's major disadvantage is its significant side effects, such as diarrhea, dizziness, drowsiness, indigestion, lightheadedness, loss of appetite, nausea, sore mouth, throat or tongue, and vomiting. These side effect associated with systemic delivery of the drug were not observed with the topical minocycline compositions of the present invention. 3. Tolerability. Another disadvantage of Oral Minocycline's is skin irritation. In contrast, as stated above, the tolerability of topical minocycline was excellent.

It has been noted that combining a topical antibiotic directed to the treatment of acne vulgaris together with a waterless oily carrier, comprising foam adjuvants has a skin conditioning effect as well as a therapeutic effect which results in restoration of skin integrity, freshness and supple appearance. The fact that the compositions are also free of surfactants and alcohols which are known skin irritants make these formulations suitable for sensitive and damaged skin. Moreover, anti-inflammatory attributes of the formulations seem to be indicated by the low occurrence of erythema, dryness, and peeling and that they disappeared during treatment.

Thus, topical minocycline foam offers an easy, safe and an effective solution for the topical treatment of acne vulgaris. The ease of use, with once daily dosing, as well as its broad spectrum of activity treating both inflammatory and non-inflammatory lesions, quick onset of clinical effect, high tolerability and the low frequency of adverse effects and high patient satisfaction make it an attractive choice. In addition to application as a foam the results can be extrapolated for use with gel and liquid gel delivery formats. Further, the results of the present study suggest that the topical minocycline formulation leads to a significantly better as well as earlier response on the inflammatory acne lesions and was well tolerated by patients.

The study thus demonstrates that the topical minocycline formulation of the present invention is more effective in reducing both inflammatory as well as the non-inflammatory acne lesions than the marketed formulation.

The results of this study indicate that treatment of moderate to severe acne with minocycline foam was associated with a favorable efficacy, safety and tolerability profile in the concentrations and total exposures investigated. A clinically significant effect was observed for daily application of the minocycline foam with an onset as early as 3 weeks of treatment, with the effect most notably shown in subjects treated with 4% minocycline foam on both inflammatory and non-inflammatory lesions.

### 10. Compositions

Study medication was provided as pressurized foam formulation containing suspended micronized minocycline hydrochloride 1% and 4% for topical administration. Excipients were used at concentrations within those indicated in the FDA Inactive Ingredients Database: (www.accessdata.fda.gov/scripts/cder/iig/index.cfm). Matching placebo was provided as pressurized foam formulation with no active ingredient, for topical administration.

The formulation was manufactured by applicant, under good manufacturing practice (GMP) and provided in labeled aerosol containers according to local regulations.

The below compositions, used in the clinical study, were prepared according to the manufacturing procedures detailed in Example 1.

TABLE 10.1
Formulations: Placebo, 1% Minocycline and 4% Minocycline

| | | MCH 1% | MCH 4% |
|---|---|---|---|
| Formulations | Placebo | (MCH244 1%) | (MCH244 4%) |
| Ingredients | % w/w | % w/w | % w/w |
| Light Mineral oil | 5.499 | 4.44 | 1.11 |
| Cyclomethicone | 5.00 | 5.00 | 5.00 |
| Coconut oil | 23.60 | 23.60 | 23.60 |
| Soybean oil | 50.00 | 50.00 | 50.00 |
| Hydrogenated castor oil | 2.00 | 2.00 | 2.00 |
| Beeswax | 2.00 | 2.00 | 2.00 |
| Myristyl alcohol | 2.50 | 2.50 | 2.50 |
| Cetostearyl alcohol | 3.50 | 3.50 | 3.50 |
| Stearyl alcohol | 1.50 | 1.50 | 1.50 |
| Behenyl alcohol | 1.10 | 1.10 | 1.10 |
| Fumed Silica (SiO2) | 0.25 | 0.25 | 0.25 |
| Stearic acid | 3.00 | 3.00 | 3.00 |
| Minocycline HCl (micronized | — | 1.11 | 4.44 |
| 90% potency) | | | |
| Quinoline yellow WS | 0.05 | — | — |
| (D&C yellow 10) | | | |
| Quinoline yellow SS | 0.001 | — | — |
| (D&C yellow 11) | | | |
| Total | 100 | 100 | 100 |
| Propellant AP-70 | 12.00 | 12.00 | 12.00 |

All inactive ingredients used in the formulation are intended for topical use and listed in the current FDA Inactive Ingredient Database; concentrations used do not exceed the maximum concentrations given in Database.

### D&C Yellow #10

The color additive D&C Yellow No. 10 is a mixture of the sodium salts of the mono- and disulfonic acids of 2-(2-quinolinyl)-1H-indene-1,3 (2H)-dione consisting principally of the sodium salts of 2-(2,3-dihydro-1,3-dioxo-1H-indene-2-yl)-6-quinolinesulfonic acid and 2-(2,3-dihydro-1,3-dioxo-1H-indene-2-yl)-8-quinolinesulfonic acid with lesser amounts of the disodium salts of the disulfonic acids of 2-(2-quinolinyl)-1H-indene-1,3(2H)-dione (CAS Reg. No. 8004-92-0). D&C Yellow No. 10 is manufactured by condensing quinaldine with phthalic anhydride to give the unsulfonated dye, which is then sulfonated with oleum.

D&C Yellow #10 is approved by the FDA for use in drugs & cosmetics and is listed in the Color Additive Status List published by the FDA in 2009.

### D&C Yellow #11

The color additive D&C Yellow No. 11 is principally 2-(2-quinolyl)-1,3-indandione (CAS Reg. No. 8003-22-3). D&C Yellow #11 is approved by the FDA for use in externally applied drugs & cosmetics and is listed in the Color Additive Status List published by the FDA in 2009.

### 11. Stability

The achievement of a long term stable foamable formulation of tetracycline antibiotics described herein, was a major challenge and required both extensive research and creativity.

The following examples illustrates the chemical stability of minocycline HCl (“MCH”) and doxycycline hyclate in oleaginous formulations, MCH244 and DOX244 as described in Tables 11.1 to 11.6 below. In an accelerated stability study, samples were stored at 40° C., and the concentrations of minocycline HCl and doxycycline hyclate were determined by UPLC. The stability test results following 2 months, 3 months, 6 months, 9 months, 12 months, 18 months of storage are shown herein below.

Samples of 244 1% and 4% were stored at 25° C. and 40° C. in order to test physical and chemical stability.

### 1. Inspection of Formulation in Glass Bottles

The use of pressurized glass bottles enables the inspection of formulations for homogeneity in the presence of propellant. Following 18 months of storage at 25° C. the formulation was found to be re-dispersible, i.e., homogeneous following slight shaking.

### 2. Stability Following Storage at 25° C. and 40° C.

Storage at 25° C. and 40° C. for 18 months revealed almost no change in the Minocycline concentration. Test results for chemical stability of minocycline following storage for up to 18 months at 25° C. and 40° C. are summarized in Table 4 and Table 5. There was practically no degradation of 244 1% and 4% minocycline following 18 months at 25° C. and also following 9 months at 40° C. These stability results indicate shelf life of more than two years at ambient temperature. Test results for chemical stability of doxycycline following storage for up to 9 months at 25° C. and 40° C. are summarized in Tables 7-9. There was practically no degradation of doxycycline following 6 months at 25° C. and at 40° C. These stability results likewise indicate a long shelf life of more than two years at ambient temperature. In one or more embodiments the tetracycline composition has a shelf life of at least 6 months, or at least 9 months, or at least 12 months or at least 15 months, or at least 18 months or at least 21 months or at least 24 months at ambient temperature. In one or more embodiments the tetracycline composition has a shelf life of at least 6 months, or at least 9 months, or at least 12 months or at least 15 months, or at least 18 months or at least 21 months or at least 24 months at 25° C. In one or more embodiments the tetracycline composition has a shelf life of at least 1 month, or at least 3 months, or at least 3 months or at least 6 months, or at least 9 months or at least 12 months 40° C.

TABLE 11.1
Minocycline content in 244 1% following
storage for 18 months at 25° C. and 40° C.

| | Minocycline content (% w/w) | | | | | |
|---|---|---|---|---|---|---|
| Temp | T = 0 | 3 M | 6 M | 9 M | 12 M | 18 M |
| 25° C. | 1.001 | NM | 0.986 | 1.007 | 0.972 | 0.959 |
| 40° C. | 1.001 | 1.002 | 0.983 | 0.965 | NM | NM |

NM = not measured

TABLE 11.2
Minocycline content in FXFM244 4% following storage for
18 months at 25° C. and 40° C. (Lot MCH-244-100825)

| | Minocycline content (% w/w) | | | | | |
|---|---|---|---|---|---|---|
| Temp | T = 0 | 3 M | 6 M | 9 M | 12 M | 18 M |
| 25° C. | 1.012 | NM | 0.998 | 0.998 | 0.972 | 0.925 |
| 40° C. | 1.012 | 0.963 | 1.009 | 0.978 | NM | NM |

### Minocycline Physical Stability:

The results for physical stability following storage at 25° C. and 40° C. for 18 months were as follows:

Foam quality: Conformed to the foam quality specification following 9 months storage at 40° C.

Odor: Conformed to the specification and showed no odor following storage at 40° C. for 9 months.

Color: The color of the formulation remained light slightly changed to grey yellow following storage at 40° C. for 9 months. No change was observed at 25° C.

Shakability: Conformed to specifications following storage at 40° C. for 9 months.

Density: No significant change in density was found after storage at 40° C. for 9 months.

Collapse time: No change in foam collapse time (the time for the foam to reach half of its initial height) was found in any of the formulation samples tested after storage for 9 months at 40° C.

Microscopic observations: No significant change in the microscopic appearance was noted following storage at 40° C. for 9 months.

Corrosion and deterioration: The coated aluminum surfaces of the can and valve and the plastic housing of the valve appeared fully intact and showed no signs of corrosion or deterioration. No changes in color or deformation were observed.

### Doxycycline Physical Stability:

The results for physical stability following storage at 25° C. for 9 months and 40° C. for 6 months were as follows:
Foam quality: At least good quality.
Odor: Showed no or very faint odor.
Collapse time: At least 100 seconds.

### Production GMP Compliance

For the purpose of clinical supplies, the productions of the compositions were performed according to the principles of current good manufacturing practice (c-GMP). Production conditions were aimed to ensure high quality of the product and to prevent any potential cross contamination. The production site was certified by the Israel Ministry of Health as suitable for GMP production and supply of small clinical batches for Phase I and IIa clinical trials.

The below composition was prepared according to the manufacturing procedures detailed in Example 1.

TABLE 11.3
Formulation of DOX-244-111123

| Ingredient Name | % W/W |
|---|---|
| Coconut oil | 23.60 |
| Mineral oil light | 4.35 |
| soybean oil | 50.00 |
| stearic acid | 3.00 |
| behenyl alcohol | 1.10 |
| hydrogenated castor oil | 2.00 |
| Bees wax | 2.00 |
| Stearyl alcohol | 1.50 |
| Cetostearyl alcohol | 3.50 |
| Myristyl alcohol | 2.50 |
| Cyclomethicone | 5.00 |
| Silicon dioxide | 0.25 |
| Doxycycline Hyclate | 1.20 |

TABLE 11.4
Doxycycline % content in DOX-244-111123 PF following
storage for 9 months at 5° C. 25° C. 40° C. and 50° C.

| | Doxycycline content (% w/w) | | | | | | | | | | |
|---|---|---|---|---|---|---|---|---|---|---|---|
| | | 1 M | | | 2 M | | 3 M | | 6 M | | 9 M |
| Batch/ | | 5° | 25° | 50° | 25° | 50° | 25° | 40° | 25° | 40° | 25° |
| Sample name | T = 0 | C. | C. | C. | C. | C. | C. | C. | C. | C. | C. |
| DOX-244- | 1.0220 | 1.031 | 1.022 | NM | NM | NM | 1.010 | 1.031 | 1.017 | 1.025 | 1.053 |
| 111123 PF | | | | | | | | | | | |
| DOX-244- | 1.0800 | 1.098 | 1.080 | 1.060 | NM | 1.045 | 1.082 | 1.046 | 1.046 | 1.028 | 1.091 |
| 111123 PFF | | | | | | | | | | | |

TABLE 11.5
Stability of Doxycyline Foam at 25° C. and 40° C.
%³ Doxycycline in DOX244 foam product

| Months | 40° C. (foam) | 25° C. (foam) |
|---|---|---|
| 0 | 102.2 | 102.2 |
| 1 | | 102.2 |
| 2 | | |
| 3 | 103.1 | 101.0 |
| 6 | 102.5 | 101.7 |
| 9 | | 105.3 |

³The percentages are derived from the PF FIGURES in Table 15.
Note
1.2% doxycycline hyclate is equivalent to 1.0176%. doxycycline based on USP

TABLE 11.6
Degradation of Doxycycline at 5° C. 25° C. 40° C. and 50° C.

| Degradation | Batch/Sample | DOX-244- | DOX-244- |
|---|---|---|---|
| product w/w | name | 111123 PF | 111123 PF |
| T0 | RRT 0.75 | 0.003 | 0.004 |
| | RRT 0.85 | 0.010 | 0.011 |
| 1M | 5° C. RRT 0.75 | 0.003 | 0.003 |
| | 5° C. RRT 0.85 | 0.010 | 0.010 |
| | 25° C. RRT 0.75 | 0.003 | 0.003 |
| | 25° C. RRT 0.85 | 0.010 | 0.010 |
| | 50° C. RRT 0.75 | NM | 0.003 |
| | 50° C. RRT 0.85 | NM | 0.01 |
| 2M | 50° C. RRT 0.75 | NM | 0.003 |
| | 50° C. RRT 0.85 | NM | 0.009 |
| 3M | 25° C. RRT 0.75 | 0.003 | 0.004 |
| | 25° C. RRT 0.85 | 0.01 | 0.011 |
| | 40° C. RRT 0.75 | 0.003 | 0.003 |
| | 40° C. RRT 0.85 | 0.01 | 0.01 |
| 6M | 25° C. RRT 0.75 | 0.003 | 0.003 |
| | 25° C. RRT 0.85 | 0.01 | 0.01 |
| | 40° C. RRT 0.75 | 0.003 | 0.003 |
| | 40° C. RRT 0.85 | 0.01 | 0.01 |
| 9M | 25° C. RRT 0.75 | 0.003 | 0.003 |
| | 25° C. RRT 0.85 | 0.009 | 0.01 |

### Example 4—Ex-Vivo Studies on Minocycline Skin Penetration

### 1. Study Synopsis

The following procedure was used to assess skin penetration and specific targeting of foam samples. Two mg/cm² of the minocycline foam formulation, according to COLIPA and United States standards, were applied on pig ear skin. A skin area of 10×8 cm² was marked with a permanent marker. A hundred and sixty mg of the formulation was applied with a syringe and distributed homogeneously with a gloved finger. Subsequent to application the substance was left on the skin for an hour. A biopsy was taken upon completion of penetration period. Histological sections of the hair follicles were prepared subsequent to biopsy. Pictures were obtained using fluorescence microscopy, reflectance and fluorescent images being superimposed.

### 2. Study Results

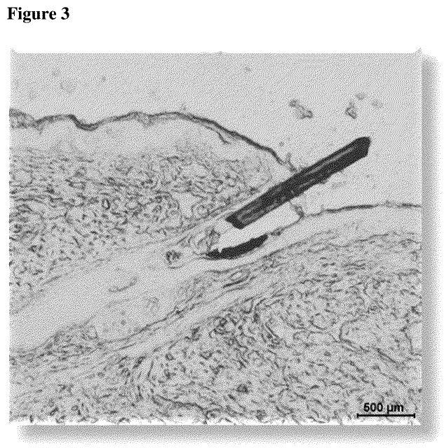

Ex-vivo studies surprisingly revealed that minocycline penetrated the skin and hair follicle area and probably into the sebaceous gland. As clearly shown in FIG. 3, Minocycline penetrated the hair follicles (dark areas) while the surrounding tissue and hair remained intact (light areas).

It is postulated, without being bound by any theory, that the use of a hydrophobic oil based foam vehicle may contribute to cutaneous bioavailability of minocycline, including the achievement of therapeutic levels of minocycline in the pilosebaceous unit. The penetration and targeting to the pilosebaceous unit may be facilitated by the hydrophobic nature of the pilosebaceous gland and the hydrophobic nature of the vehicle and the hydrophobic region of the minocycline molecule.

1-34. (canceled)

35. A method of treating or alleviating rosacea,
comprising topically administering a hydrophobic foam once daily to a target area on the skin or mucosa of a human subject having rosacea, wherein the hydrophobic foam is formed from a pre-foam formulation and a liquefied or compressed gas propellant, wherein the pre-foam formulation comprises:
a) about 60% to about 99% by weight of the pre-foam formulation of at least one hydrophobic solvent comprising a soybean oil;
b) a wax comprising a hydrogenated castor oil;
c) a fatty alcohol having a carbon chain length of 14 to 22 carbons, a fatty acid having a carbon chain length of 12 to 28 carbons, or mixtures of any two or more thereof; and
d) a therapeutically effective amount of a minocycline or a salt thereof; wherein the wax and/or fatty alcohol constitute about 1% to about 22% by weight of the pre-foam formulation;
wherein the pre-foam formulation has a water activity (Aw) value of less than about 0.9; and
wherein the pre-foam formulation is surfactant free.

36. A hydrophobic foam formed from a composition comprising a pre-foam formulation and a liquefied or compressed gas propellant, wherein the pre-foam formulation comprises:
a) about 60% to about 99% by weight of the pre-foam formulation of at least one hydrophobic solvent comprising a soybean oil;
b) a wax comprising a hydrogenated castor oil;
c) a fatty alcohol having a carbon chain length of 14 to 22 carbons, a fatty acid having a carbon chain length of 12 to 28 carbons, or mixtures of any two or more thereof; and
d) a therapeutically effective amount of a minocycline or a salt thereof;
wherein the wax and/or fatty alcohol constitute about 1% to about 22% by weight of the pre-foam formulation;
wherein the pre-foam formulation has a water activity (Aw) value of less than about 0.9; and
wherein the pre-foam formulation is surfactant free.

37. The method of claim 35, where the Aw value is less than about 0.5.

38. The method of claim 35, wherein the hydrophobic solvent comprises two or more of a soybean oil, a coconut oil, a cyclomethicone, and a mineral oil.

39. The method of claim 35, wherein the wax comprises two or more of a hydrogenated castor oil, a beeswax, a paraffin wax, and a wax that has a melting point of more than 40° C.
